(19) United States
(12) Patent Application Publication
(10) Pub. No.: US 2024/0174746 A1
(43) Pub. Date: May 30, 2024

(54) ANTI-TMEM106B ANTIBODIES AND METHODS OF USE THEREOF

(71) Applicant: ALECTOR LLC, South San Francisco, CA (US)

(72) Inventors: Eric BROWN, South San Francisco, CA (US); Angie Grace YEE, San Francisco, CA (US); Tina SCHWABE, San Francisco, CA (US); Herve RHINN, San Francisco, CA (US); Arnon ROSENTHAL, Woodside, CA (US)

(21) Appl. No.: 18/551,162

(22) PCT Filed: Mar. 17, 2022
(86) PCT No.: PCT/US2022/020785

§ 371 Date: Sep. 18, 2023

Related U.S. Application Data
(60) Provisional application No. 63/162,849, filed on Mar. 18, 2021.

(51) Int. Cl. C07K 16/28; A61K 39/00

(52) U.S. Cl. CPC C07K 16/28; A61K 2039/505; C07K 2317/33; C07K 2317/56; C07K 2317/76

(57) ABSTRACT

The present disclosure is generally directed to compositions that include antibodies, e.g., monoclonal antibodies, antibody fragments, etc., that specifically bind a TMEM106B polypeptide, e.g., a mammalian TMEM106B or human TMEM106B, and use of such compositions in preventing, reducing risk, or treating an individual in need thereof.

### CROSS-REFERENCE TO RELATED APPLICATIONS

This application claim the priority benefit of U.S. Provisional Application No. 63/162,849, filed Mar. 18, 2021, which is herein incorporated by reference in its entirety.

### SUBMISSION OF SEQUENCE LISTING ON ASCII TEXT FILE

The content of the following submission of ASCII text file is incorporated herein by reference in its entirety: a computer readable form (CRF) of the Sequence Listing (file name: 4503_014PC01_Seqlisting_ST25.txt date of creation: Mar. 16, 2022; size: 134,365 bytes).

### FIELD OF THE PRESENT DISCLOSURE

The present disclosure relates to anti-TMEM106B antibodies and therapeutic uses of such antibodies.

### BACKGROUND OF THE PRESENT DISCLOSURE

Transmembrane protein 106B (TMEM106B) is a type 2 single pass transmembrane glycoprotein residing primarily within the membrane of late endosome and lysosomes. (See, e.g., Lang et al., 2012, J Biol Chem, 287:19355-19365; Chen-Plotkin et al., 2012, J Neurosci, 32:11213-11227; Brady et al., 2013, Human Molecular Genetics, 22:685-695.) TMEM106B is widely expressed in human tissue, and of particular interest expressed in neurons, glial cells, and endothelial and peri-vascular cells in the brain. TMEM106B is highly conserved in mammals, with the human protein sharing 99% sequence identity with the cynomolgus variant and 97% sequence identify with the murine ortholog.

TMEM106B has a cytoplasmic domain predicted to range from amino acid residues 1-92 (of human TMEM106B; SEQ ID NO:1), a transmembrane domain predicted to range from amino acid residues 96-117, and a luminal domain predicted to range from amino acid residues 118-274. Five sequence motifs of post-translational N-glycosylation sites (N-X-T/S) span its luminal domain. Simple glycans are added to three of the asparagine residues (N145, N151, and N164) and are not critical for TMEM106B localization. Complex glycans are added to the most C-terminal motifs at N183 and N256; loss of complex glycans on N183 impairs TMEM106B forward transport to endosomes/lysosomes and results in endoplasmic retention. Additionally, N256 complex glycosylation is necessary for proper TMEM106B sorting. (See, e.g., Nicholson and Rademakers, 2016, Acta Neuropathol, 132:639-651.)

The function of TMEM106B has not been fully characterized. Recent reports have indicated a role of TMEM106B in dendrite branching, morphogenesis, and maintenance by inhibiting trafficking of lysosomes along dendrites. (See, e.g., Brady et al., 2013, Human Molecular Genetics, 126:696-698; Schwenk et al., 2014, EMBO J, 33:450-467; Clayton et al., 2018, Brain 141(12): 3428-3442.)

TMEM106B has been shown to interact with various proteins, including without limitation progranulin protein (GRN), other TMEM106 protein family members, such as TMEM106A and TMEM106C, clathrin heavy chain (CLTC), the μ1 subunit of adipocyte protein 2 (AP2M1), charged multi-vesicular body protein 2b (CHMP2B), microtubule-associated protein 6 (MAP6), lysosomal-associated membrane protein 1 (LAMP1), and vacuolar-ATPase subunit accessory protein 1 (v-ATPase Ap1).

TMEM106B has been genetically linked to various disorders and diseases, in particular neurodegenerative disorders. Such disorders include, without limitation, conditions characterized by the presence of pathological TDP-43 inclusions (i.e., TDP-43 proteinopathies; transactive response DNA binding protein 43), Frontotemporal lobar degeneration (FTLD), FTLD with TDP-43 inclusions (FTLD-TDP), including FTLD-TDP caused by progranulin (GRN) or C90rf72 mutations, TDP-43 proteinopathies, Alzheimer's disease. Lewy body dementia (LBD), hippocampal sclerosis (HpScl), hippocampal sclerosis of aging (HS-Aging), hypomyelinating leukodystrophies, and cognitive impairment in various disorders, such as amyotrophic lateral sclerosis (ALS). TMEM106B has also been linked to metastasis in non-small cell lung cancer (Kundu et al., 2016; Nature Commun. 2018; 9: 2731, Cancer Research, Proceedings of the 107^{th} Annual Meeting of the American Association for Cancer Research, abstract no. 688). TMEM106B has also been linked to chronic traumatic encephalopathy (CTE)-related neuropathology and dementia in CTE patients, including changes in AT8 tau deposition, CD68 cell density and PSD-95 concentration (Cherry et al., 2018, Acta Neuropathol Commun. 6:115).

Accordingly, there is a need for therapies targeting TMEM106B, including therapeutic antibodies that specifically bind TMEM106B, and/or therapies that are capable of inhibiting the activity of TMEM106B, such as by reducing TMEM106B protein levels or function or by blocking or reducing the binding of TMEM106B to one or more of its ligands or binding partners, or otherwise modulate the effective concentration of one or more of its ligands or binding partners, in order to treat various diseases, disorders, and conditions associated with TMEM106B activity.

All references cited herein, including patent applications and publications, are hereby incorporated by reference in their entirety.

### SUMMARY OF THE PRESENT DISCLOSURE

The present disclosure is generally directed to anti-TMEM106B antibodies and methods of using such antibodies. The methods provided herein find use in preventing, reducing risk, or treating an individual having a neurodegenerative disease, disorder, or condition. In some embodiments, the present invention provides a method for preventing, reducing risk, or treating an individual having a disease, disorder, or injury selected from the group consisting of a neurodegenerative disorder, a disorder characterized by the presence of TDP-43 inclusions, a TDP-43 proteinopathy, inflammatory cell debris or protein aggregates, abnormal circulating myeloid cells, unhealthy aging, frontotemporal lobar degeneration (FTLD), frontotemporal dementia (FTD), FTD with progranulin mutations, FTD with C90rf72 mutations, frontotemporal lobar degeneration with TDP-43 inclusions, hippocampal sclerosis (HpScl), hippocampal sclerosis of aging (HS-Aging), Alzheimer's disease, Lewy body dementia, cognitive impairment, age related cognitive impairment, age related brain atrophy, age-associated traits, including without limitations inflammation, neuronal loss, and cognitive deficits, such as cognitive defects in the absence of known brain disease, including cognitive deficits of the frontal cerebral cortex of older individuals, cognitive impairment in amyotrophic lateral sclerosis, cognitive impairment in chronic traumatic encephalopathy (CTE), diseases, disorders, and conditions associated with over expression or increased activity of TMEM106B, a hypomyelinating disorder, cancer, and metastasis the method comprising administering to an individual in need thereof a therapeutically effective amount of an anti-TMEM106B antibody.

Accordingly, certain aspects of the present disclosure relate to an isolated (e.g., monoclonal) anti-TMEM106B antibody, wherein the anti-TMEM106B antibody has a property selected from the group consisting of: decreasing cellular levels of TMEM106B, decreasing intracellular levels of TMEM106B, inhibiting or reducing the interaction between TMEM106B and one or more of its ligand or binding proteins, and any combination thereof. The ability of an antibody to inhibit the interation between TMEM106B and a ligand or binding protein can be determined by co-immunoprecipitation of the ligand or binding protein of TMEM106B protein in the presence and absence of an anti-TMEM106B antibody, followed by Western blot detection of the ligand or binding partner. A decrease in the detection of the ligand or binding partner in the presence of the anti-TMEM106B antibody as compared to in the absence of the anti-TMEM106B antibody indicates that the antibody inhibits the interaction between TMEM106B and the ligand or binding partner.

In certain embodiments that may be combined with any of the embodiments provided herein, the antibody decreases cell surface levels of TMEM106B, decreases intracellular levels of TMEM106B, decreases total levels of TMEM106B, decreases endosomal levels of TMEM106B, decreases lysosomal levels of TMEM106B, or any combination thereof. In certain embodiments that may be combined with any of the embodiments provided herein, the anti-TMEM106B antibody induces TMEM106B degradation, TMWM106B cleavage, TMEM106B internalization, TMEM106B down regulation, or any combination thereof. In certain embodiments that may be combined with any of the preceding embodiments, the anti-TMEM106B antibody decreases cellular levels of TMEM106B in vivo. In certain embodiments that may be combined with any of the embodiments provided herein, the anti-TMEM106B antibody decreases cellular levels of TMEM106B in brain. In certain embodiments that may be combined with any of the embodiments provided herein, the anti-TMEM106B antibody decreases cellular levels of TMEM106B in one or more peripheral organs. In certain embodiments that may be combined with any of the embodiments provided herein, the anti-TMEM106B antibody decreases cellular levels of TMEM106B in brain, one or more peripheral organs, or any combination thereof. In certain embodiments that may be combined with any of the embodiments provided herein, the anti-TMEM106B antibody decreases cellular levels of TMEM106B in microglia. In certain embodiments that may be combined with any of the embodiments provided herein, the anti-TMEM106B antibody decreases cellular levels of TMEM106B in neurons.

In certain embodiments that may be combined with any of the embodiments provided herein, the anti-TMEM106B antibody inhibits or reduces one or more interactions between TMEM106B and progranulin protein, other TMEM106 protein family members, such as TMEM106B and TMEM106C, clathrin heavy chain (CLTC), the μ1 subunit of adipocyte protein 2 (AP2M1), CHMP2B, microtubule-associated protein 6 (MAP6), lysosomal-associated membrane protein 1 (LAMP1), vacuolar-ATPase subunit accessory protein 1, or any protein or polypeptide that modulates the function of TMEM106B.

In certain embodiments that may be combined with any of the embodiments provided herein, an anti-TMEM106B antibody of the present disclosure binds a discontinuous TMEM106B epitope. In certain embodiments that may be combined with any of the preceding embodiments, the discontinuous TMEM106B epitope comprises two or more peptides, three or more peptides, four or more peptides, five or more peptides, six or more peptides, seven or more peptides, eight or more peptides, nine or more peptides, or 10 or more peptides. In certain embodiments that may be combined with any of the embodiments provided herein, each of the peptides comprise five or more, six or more, seven or more, eight or more, nine or more, 10 or more, 11 or more, 12 or more, 13 or more 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, or 30 or more amino acid residues of the amino acid sequence of SEQ ID NO:1, of the amino acid sequence of SEQ ID NO:2, or of the amino acid sequence of SEQ ID NO:3; or five or more, six or more, seven or more, eight or more, nine or more, 10 or more, 11 or more, 12 or more, 13 or more 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, or 30 or more amino acid residues on a mammalian TMEM106B protein corresponding to the amino acid sequence of SEQ ID NO:1, to the amino acid sequence of SEQ ID NO:2, or to the amino acid sequence of SEQ ID NO:3.

In certain embodiments that may be combined with any of the embodiments provided herein, an anti-TMEM106B antibody of the present disclosure binds to a conformational epitope of TMEM106B.

In certain embodiments that may be combined with any of the embodiments provided herein, an anti-TMEM106B antibody of the present disclosure competes with one or more reference anti-TMEM106B antibodies comprising the V_{H} and V_{L} of the antibody selected from the group consisting of TM-54, TM-56, TM-59, TM-60, TM-61, TM-62, TM-63, TM-64, TM-65, TM-66, TM-68, TM-69, TM-70, TM-71, TM-72, TM-73, TM-74, TM-75, TM-76, TM-77, TM-78, TM-79, TM-80, TM-81, TM-82, TM-83, TM-84, TM-85, TM-86, TM-87, TM-88, TM-89, TM-90, TM-91, TM-92, TM-93, and TM-94, and any combination thereof.

Other aspects of the present disclosure relate to an isolated (e.g., monoclonal) anti-TMEM106B antibody, wherein the anti-TMEM106B antibody comprises at least one, two, three, four, five, or six HVRs of an antibody selected from the group consisting of: TM-54, TM-56, TM-59, TM-60, TM-61, TM-62, TM-63, TM-64, TM-65, TM-66, TM-68, TM-69, TM-70, TM-71, TM-72, TM-73, TM-74, TM-75, TM-76, TM-77, TM-78, TM-79, TM-80, TM-81, TM-82, TM-83, TM-84, TM-85, TM-86, TM-87, TM-88, TM-89, TM-90, TM-91, TM-92, TM-93, and TM-94. In some embodiments, the anti-TMEM106B antibody comprises the six HVR (e.g., as shown in Tables 2 and 3 below) of the antibody selected from the group consisting of TM-54, TM-56, TM-59, TM-60, TM-61, TM-62, TM-63, TM-64, TM-65, TM-66, TM-68, TM-69, TM-70, TM-71, TM-72, TM-73, TM-74, TM-75, TM-76, TM-77, TM-78, TM-79, TM-80, TM-81, TM-82, TM-83, TM-84, TM-85, TM-86, TM-87, TM-88, TM-89, TM-90, TM-91, TM-92, TM-93, and TM-94.

Other aspects of the present disclosure relate to an isolated (e.g., monoclonal) anti-TMEM106B antibody which binds essentially the same TMEM106B epitope as a reference anti-TMEM106B antibody comprising the V_{H} and V_{L} (e.g., as shown in Table 4 below) of the antibody selected from the group consisting of: TM-54, TM-56, TM-59, TM-60, TM-61, TM-62, TM-63, TM-64, TM-65, TM-66, TM-68, TM-69, TM-70, TM-71, TM-72, TM-73, TM-74, TM-75, TM-76, TM-77, TM-78, TM-79, TM-80, TM-81, TM-82, TM-83, TM-84, TM-85, TM-86, TM-87, TM-88, TM-89, TM-90, TM-91, TM-92, TM-93, and TM-94.

In some embodiments of the present disclosure, an anti-TMEM106B antibody binds to one or more amino acids within amino acid residues 151-165 and/or 185-195. In some embodiments of the present disclosure, an anti-TMEM106B antibody binds to one or more amino acids within amino acid residues 59-73, 80-90, 139-149, and/or 248-258 of human TMEM106B (SEQ ID NO:1). In some embodiments, an anti-TMEM106B antibody binds to one or more amino acids within amino acid residues 5-19, 156-161, 202-207, and/or 219-233 of human TMEM106B (SEQ ID NO:1). In some embodiments, an anti-TMEM106B antibody binds to one or more amino acids within amino acid residues 126-140, 185-195, and/or 260-274 of human TMEM106B (SEQ ID NO:1). In some embodiments, an anti-TMEM106B antibody binds to one or more amino acids within amino acid residues 202-212 of human TMEM106B (SEQ ID NO:1). In some embodiments, an anti-TMEM106B antibody binds to one or more amino acids within amino acid residues 151-161 and/or 223-233 of human TMEM106B (SEQ ID NO:1). In some embodiments, an anti-TMEM106B antibody binds to one or more amino acids within amino acid residues 59-69, 143-153, and/or 223-228 of human TMEM106B (SEQ ID NO:1). In some embodiments, an anti-TMEM106B antibody binds to one or more amino acids within amino acid residues 133-145 and/or 198-212 of human TMEM106B (SEQ ID NO:1). In some embodiments, an anti-TMEM106B antibody binds to one or more amino acids within amino acid residues 52-62, 64-75, and/or 223-228 of human TMEM106B (SEQ ID NO:1).

In certain embodiments that may be combined with any of the embodiments provided herein, the anti-TMEM106B antibody further inhibits interaction between TMEM106B and one or more of its ligands, signaling proteins or binding proteins by: a) reducing the effective levels of TMEM106B available for interacting with the one or more ligands or binding proteins; b); blocking one or more of the sites on TMEM106B required for interaction with the one or more ligands or binding proteins; c) preventing one or more posttranslational events on TMEM106B that are required for interaction with the one or more ligands or binding proteins and/or for correct processing and/or subcellular localization of TMEM106B; d) inducing degradation of TMEM106B; e) changing the conformation of TMEM106B, or both. In certain embodiments that may be combined with any of the embodiments provided herein, the anti-TMEM106B antibody binds specifically to human TMEM106B, mouse TMEM106B, cynomolgus (cyno) TMEM106B, or a combination thereof. In certain embodiments that may be combined with any of the embodiments provided herein, the anti-TMEM106B antibody is a human antibody, a humanized antibody, a bispecific antibody, a monoclonal antibody, a multivalent antibody, a conjugated antibody, or a chimeric antibody. In certain embodiments that may be combined with any of the embodiments provided herein, the anti-TMEM106B antibody is a bispecific antibody recognizing a first antigen and a second antigen. In certain embodiments that may be combined with any of the embodiments provided herein, the first antigen is TMEM106B and the second antigen is an antigen facilitating transport across the blood-brain-barrier. In certain embodiments that may be combined with any of the embodiments provided herein, the second antigen is selected from the group consisting of TMEM106B, transferrin receptor (TR), insulin receptor (HIR), insulin-like growth factor receptor (IGFR), low-density lipoprotein receptor related proteins 1 and 2 (LPR-1 and 2), diphtheria toxin receptor, CRM197, a llama single domain antibody, TMEM 30(A), a protein transduction domain, TAT, Syn-B, penetratin, a poly-arginine peptide, an angiopep peptide, basigin, Glut1, and CD98hc, and ANG1005.

In some embodiments that may be combined with any of the embodiments provided herein, the antibody is a monoclonal antibody. In some embodiments that may be combined with any of the preceding embodiments, the antibody is of the IgG class, the IgM class, or the IgA class. In some embodiments, the antibody is of the IgG class and has an IgG1, IgG2, or IgG4 isotype. In certain embodiments that may be combined with any of the preceding embodiments, the anti-TMEM106B antibody is an antibody fragment that binds to an epitope comprising amino acid residues on human TMEM106B or a mammalian TMEM106B protein. In certain embodiments that may be combined with any of the embodiments provided herein, the fragment is a Fab, Fab′, Fab′-SH, F(ab′)2, Fv, or scFv fragment. In some embodiments that may be combined with any of the preceding embodiments, the antibody is a humanized antibody or a chimeric antibody.

Other aspects of the present disclosure relate to an isolated nucleic acid comprising a nucleic acid sequence encoding the anti-TMEM106B antibody of any of the preceding embodiments. Other aspects of the present disclosure relate to a vector comprising the nucleic acid of any of the preceding embodiments. Other aspects of the present disclosure relate to an isolated host cell comprising the vector of any of the preceding embodiments. Other aspects of the present disclosure relate to a method of producing an anti-TMEM106B antibody, comprising culturing the host cell of any of the preceding embodiments so that the anti-TMEM106B antibody is produced. In certain embodiments, the method further comprises recovering the anti-TMEM106B antibody produced by the host cell. Other aspects of the present disclosure relate to an isolated anti-TMEM106B antibody produced by the method of any of the preceding embodiments. Other aspects of the present disclosure relate to a pharmaceutical composition comprising the anti-TMEM106B antibody of any of the preceding embodiments, and a pharmaceutically acceptable carrier.

Other aspects of the present disclosure relate to a method of preventing, reducing risk, or treating an individual having a disease, disorder, or injury selected from the group consisting of frontotemporal dementia, Alzheimer's disease, vascular dementia, seizures, retinal dystrophy, a traumatic brain injury, a spinal cord injury, long-term depression, atherosclerotic vascular diseases, undesirable symptoms of normal aging, dementia, mixed dementia, Creutzfeldt-Jakob disease, normal pressure hydrocephalus, amyotrophic lateral sclerosis, Huntington's disease, taupathy disease, stroke, acute trauma, chronic trauma, lupus, acute and chronic colitis, Crohn's disease, inflammatory bowel disease, ulcerative colitis, malaria, essential tremor, central nervous system lupus, Behcet's disease, Parkinson's disease, dementia with Lewy bodies, multiple system atrophy, degenerative disc disease, Shy-Drager syndrome, progressive supranuclear palsy, cortical basal ganglionic degeneration, acute disseminated encephalomyelitis, granulomartous disorders, Sarcoidosis, diseases of aging, age related macular degeneration, glaucoma, retinitis pigmentosa, retinal degeneration, respiratory tract infection, sepsis, eye infection, systemic infection, inflammatory disorders, arthritis, multiple sclerosis, metabolic disorder, obesity, insulin resistance, type 2 diabetes, tissue or vascular damage, an injury, inflammatory cell debris or protein aggregates, abnormal circulating myeloid cells, unhealthy aging, age-related cognitive impairment, age-related brain atrophy, age-associated traits, including without limitation inflammation, neuronal loss, and cognitive deficits, such as cognitive deficits in the absence of known brain disease, including cognitive deficits of the frontal cerebral cortex of an older individual and, one or more undesirable symptoms of normal aging, comprising administering to the individual a therapeutically effective amount of the anti-TMEM106B antibody of any of the preceding embodiments. Other aspects of the present disclosure relate to an anti-TMEM106B antibody of any of the preceding embodiments for use in preventing, reducing risk, or treating an individual having a disease, disorder, or injury selected from the group consisting of frontotemporal dementia, Alzheimer's disease, vascular dementia, seizures, retinal dystrophy, a traumatic brain injury, a spinal cord injury, long-term depression, atherosclerotic vascular diseases, undesirable symptoms of normal aging, dementia, mixed dementia, Creutzfeldt-Jakob disease, normal pressure hydrocephalus, amyotrophic lateral sclerosis, Huntington's disease, taupathy disease, stroke, acute trauma, chronic trauma, lupus, acute and chronic colitis, Crohn's disease, inflammatory bowel disease, ulcerative colitis, malaria, essential tremor, central nervous system lupus, Behcet's disease, Parkinson's disease, dementia with Lewy bodies, multiple system atrophy, degenerative disc disease, Shy-Drager syndrome, progressive supranuclear palsy, cortical basal ganglionic degeneration, acute disseminated encephalomyelitis, granulomartous disorders, Sarcoidosis, diseases of aging, age related macular degeneration, glaucoma, retinitis pigmentosa, retinal degeneration, respiratory tract infection, sepsis, eye infection, systemic infection, inflammatory disorders, arthritis, multiple sclerosis, metabolic disorder, obesity, insulin resistance, type 2 diabetes, tissue or vascular damage, an injury, inflammatory cell debris or protein aggregates, abnormal circulating myeloid cells, unhealthy aging, age-related cognitive impairment, age-related brain atrophy, age-associated traits, including without limitation inflammation, neuronal loss, and cognitive deficits, such as cognitive deficits in the absence of known brain disease, including cognitive deficits of the frontal cerebral cortex of older individual, and one or more undesirable symptoms of normal aging. Other aspects of the present disclosure relate to an anti-TMEM106B antibody of any of the preceding embodiments for use in preventing or reducing metastasis. Other aspects of the present disclosure relate to an anti-TMEM106B antibody of any of the preceding embodiments for use in preventing, reducing risk, or treating an individual having cancer.

Other aspects of the present disclosure relate to use of an anti-TMEM106B antibody of any of the preceding embodiments in the manufacture of a medicament for preventing, reducing risk, or treating an individual having a disease, disorder, or injury selected from the group consisting of frontotemporal dementia, Alzheimer's disease, vascular dementia, seizures, retinal dystrophy, a traumatic brain injury, a spinal cord injury, long-term depression, atherosclerotic vascular diseases, undesirable symptoms of normal aging, dementia, mixed dementia, Creutzfeldt-Jakob disease, normal pressure hydrocephalus, amyotrophic lateral sclerosis, Huntington's disease, taupathy disease, stroke, acute trauma, chronic trauma, lupus, acute and chronic colitis, Crohn's disease, inflammatory bowel disease, ulcerative colitis, malaria, essential tremor, central nervous system lupus, Behcet's disease, Parkinson's disease, dementia with Lewy bodies, multiple system atrophy, degenerative disc disease, Shy-Drager syndrome, progressive supranuclear palsy, cortical basal ganglionic degeneration, acute disseminated encephalomyelitis, granulomartous disorders, Sarcoidosis, diseases of aging, age related macular degeneration, glaucoma, retinitis pigmentosa, retinal degeneration, respiratory tract infection, sepsis, eye infection, systemic infection, inflammatory disorders, arthritis, multiple sclerosis, metabolic disorder, obesity, insulin resistance, type 2 diabetes, tissue or vascular damage, an injury, inflammatory cell debris or protein aggregates, abnormal circulating myeloid cells, unhealthy aging, age-related cognitive impairment, age-related brain atrophy, age-associated traits, including without limitations inflammation, neuronal loss, and cognitive deficits, such as cognitive deficits in the absence of known brain disease, including cognitive deficits of the frontal cerebral cortex of older individual and one or more undesirable symptoms of normal aging. Other aspects of the present disclosure relate to a method of preventing, reducing risk, or treating an individual having a disease, disorder, or injury selected from the group consisting of frontotemporal dementia, progressive supranuclear palsy. Alzheimer's disease, vascular dementia, seizures, retinal dystrophy, amyotrophic lateral sclerosis, traumatic brain injury, a spinal cord injury, dementia, stroke, Parkinson's disease, acute disseminated encephalomyelitis, retinal degeneration, age related macular degeneration, glaucoma, multiple sclerosis, septic shock, bacterial infection, arthritis, and osteoarthritis, comprising administering to the individual a therapeutically effective amount of the anti-TMEM106B antibody of any of the preceding embodiments. Other aspects of the present disclosure relate to an anti-TMEM106B antibody of any of the embodiments provided herein for use in preventing, reducing risk, or treating an individual having a disease, disorder, or injury selected from the group consisting of frontotemporal dementia, progressive supranuclear palsy. Alzheimer's disease, vascular dementia, seizures, retinal dystrophy, amyotrophic lateral sclerosis, traumatic brain injury, a spinal cord injury, dementia, stroke, Parkinson's disease, acute disseminated encephalomyelitis, retinal degeneration, age related macular degeneration, glaucoma, multiple sclerosis, septic shock, bacterial infection, arthritis, and osteoarthritis. Other aspects of the present disclosure relate to use of an anti-TMEM106B antibody of any of the preceding embodiments in the manufacture of a medicament for preventing, reducing risk, or treating an individual having a disease, disorder, or injury selected from the group consisting of frontotemporal dementia, progressive supranuclear palsy. Alzheimer's disease, vascular dementia, seizures, retinal dystrophy, amyotrophic lateral sclerosis, traumatic brain injury, a spinal cord injury, dementia, stroke, Parkinson's disease, acute disseminated encephalomyelitis, retinal degeneration, age related macular degeneration, glaucoma, multiple sclerosis, septic shock, bacterial infection, arthritis, and osteoarthritis.

In certain embodiments that may be combined with any of the embodiments provided herein, the anti-TMEM106B antibody comprises two or more anti-TMEM106B antibodies.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### DETAILED DESCRIPTION OF THE PRESENT DISCLOSURE

The present disclosure relates to anti-TMEM106B antibodies (e.g., monoclonal antibodies); methods of making and using such antibodies; pharmaceutical compositions comprising such antibodies; nucleic acids encoding such antibodies; and host cells comprising nucleic acids encoding such antibodies.

The techniques and procedures described or referenced herein are generally well understood and commonly employed using conventional methodology by those skilled in the art, such as, for example, the widely utilized methodologies such as those described in Sambrook et al. Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003); Monoclonal Antibodies: A Practical Approach (P. Shepherd and C. Dean, eds., Oxford University Press, 2000).

### Definitions

The terms “TMEM106B” or “TMEM106B polypeptide” are used interchangeably herein refer herein to any native TMEM106B from any vertebrate source, including mammals such as primates (e.g., humans and cynomolgus (cynos)) and rodents (e.g., mice and rats), unless otherwise indicated. In some embodiments, the term encompasses both wild-type sequences and naturally occurring variant sequences, e.g., splice variants or allelic variants. In some embodiments, the term encompasses “full-length,” unprocessed TMEM106B as well as any form of TMEM106B that results from processing in the cell. In some embodiments, the TMEM106B is human TMEM106B. In some embodiments, the amino acid sequence of an exemplary TMEM106B is Uniprot Accession No: Q9NUM4 as of Jun. 27, 2006. In some embodiments, the amino acid sequence of an exemplary human TMEM106B is SEQ ID NO:1.

The terms “anti-TMEM106B antibody,” an “antibody that binds to TMEM106B,” and “antibody that specifically binds TMEM106B” refer to an antibody that is capable of binding TMEM106B with sufficient affinity such that the antibody is useful as a diagnostic and/or therapeutic agent in targeting TMEM106B. In one embodiment, the extent of binding of an anti-TMEM106B antibody to an unrelated, non-TMEM106B polypeptide is less than about 10% of the binding of the antibody to TMEM106B as measured, e.g., by a radioimmunoassay (RIA). In certain embodiments, an antibody that binds to TMEM106B has a dissociation constant (KD) of <1 μM, <100 nM, <10 nM, <1 nM, <0.1 nM, <0.01 nM, or <0.001 nM (e.g., 10⁻⁸ M or less, e.g. from 10⁻⁸ M to 10⁻¹³ M, e.g., from 10⁻⁹ M to 10⁻¹³ M). In certain embodiments, an anti-TMEM106B antibody binds to an epitope of TMEM106B that is conserved among TMEM106B from different species.

With regard to the binding of an antibody to a target molecule, the term “specific binding” or “specifically binds” or is “specific for” a particular polypeptide or an epitope on a particular polypeptide target means binding that is measurably different from a non-specific interaction. Specific binding can be measured, for example, by determining binding of a molecule compared to binding of a control molecule. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a KD for the target of about any of 10⁻⁴ M or lower, 10⁻⁵ M or lower, 10⁻⁶ M or lower, 10⁻⁷ M or lower, 10⁻⁸ M or lower, 10⁻⁹ M or lower, 10⁻¹⁰ M or lower, 10⁻¹¹ M or lower, 10⁻¹² M or lower or a KD in the range of 10⁻⁴ M to 10⁻⁶ M or 10⁻⁶ M to 10⁻¹⁰ M or 10⁻⁷ M to 10⁻⁹ M. As will be appreciated by the skilled artisan, affinity and KD values are inversely related. A high affinity for an antigen is measured by a low KD value. In one embodiment, the term “specific binding” refers to binding where a molecule binds to a particular polypeptide or epitope on a particular polypeptide without substantially binding to any other polypeptide or polypeptide epitope.

The term “immunoglobulin” (Ig) is used interchangeably with “antibody” herein. The term “antibody” herein is used in the broadest sense and specially covers monoclonal antibodies, polyclonal antibodies, multispecific antibodies (e.g., bispecific antibodies) including those formed from at least two intact antibodies, and antibody fragments so long as they exhibit the desired biological activity.

“Native antibodies” are usually heterotetrameric glycoproteins of about 150,000 Daltons, composed of two identical Light (“L”) chains and two identical heavy (“H”) chains. Each light chain is linked to a heavy chain by one covalent disulfide bond, while the number of disulfide linkages varies among the heavy chains of different immunoglobulin isotypes. Each heavy and light chain also has regularly spaced intra-chain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

For the structure and properties of the different classes of antibodies, see. e.g., Basic and Clinical Immunology, 8th Ed., Daniel P. Stites, Abba I. Terr and Tristram G. Parslow (eds.), Appleton & Lange, Norwalk, C T, 1994, page 71 and Chapter 6.

The light chain from any vertebrate species can be assigned to one of two clearly distinct types, called kappa (“κ”) and lambda (“λ”), based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated alpha (“α”), delta (“δ”), epsilon (“ε”), gamma (‘γ’), and mu (“μ”), respectively. The γ and α classes are further divided into subclasses (isotypes) on the basis of relatively minor differences in the CH sequence and function, e.g., humans express the following subclasses: IgG1, IgG2, IgG3, IgG4, IgA1, and IgA2. The subunit structures and three-dimensional configurations of different classes of immunoglobulins are well known and described generally in, for example, Abbas et al., Cellular and Molecular Immunology, 4^{th} ed. (W.B. Saunders Co., 2000).

The “variable region” or “variable domain” of an antibody, such as an anti-TMEM106B antibody of the present disclosure, refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites.

The term “variable” refers to the fact that certain segments of the variable domains differ extensively in sequence among antibodies, such as anti-TMEM106B antibodies of the present disclosure. The variable domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Immunological Interest, Fifth Edition, National Institute of Health, Bethesda, MD (1991)). The constant domains are not involved directly in the binding of antibody to an antigen, but exhibit various effector functions, such as participation of the antibody in antibody-dependent-cellular toxicity.

The term “monoclonal antibody” as used herein refers to an antibody, such as a monoclonal anti-TMEM106B antibody of the present disclosure, obtained from a population of substantially homogeneous antibodies, i.e., the individual antibodies comprising the population are identical except for possible naturally occurring mutations and/or post-translation modifications (e.g., isomerizations, amidations, etc.) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method, recombinant DNA methods, and technologies for producing human or human-like antibodies in animals that have parts or all of the human immunoglobulin loci or genes encoding human immunoglobulin sequences.

The terms “full-length antibody,” “intact antibody” or “whole antibody” are used interchangeably to refer to an antibody, such as an anti-TMEM106B antibody of the present disclosure, in its substantially intact form, as opposed to an antibody fragment. Specifically, whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” refers to a molecule other than an intact antibody that comprises a portion of an intact antibody that binds the antigen to which the intact antibody binds. Examples of antibody fragments include Fab, Fab′, F(ab′)₂ and Fv fragments; diabodies; linear antibodies (see U.S. Pat. No. 5,641,870, Example 2; Zapata et al., Protein Eng. 8(10):1057-1062 (1995)); single-chain antibody molecules and multispecific antibodies formed from antibody fragments.

Papain digestion of antibodies, such as anti-TMEM106B antibodies of the present disclosure, produces two identical antigen-binding fragments, called “Fab” fragments, and a residual “Fc” fragment, a designation reflecting the ability to crystallize readily. The Fab fragment consists of an entire light chain along with the variable region domain of the heavy chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both heavy chains held together by disulfides. The effector functions of antibodies are determined by sequences in the Fc region, the region which is also recognized by Fc receptors (FcR) found on certain types of cells.

“Functional fragments” of antibodies, such as anti-TMEM106B antibodies of the present disclosure, comprise a portion of an intact antibody, generally including the antigen binding or variable region of the intact antibody or the Fc region of an antibody which retains or has modified FcR binding capability. Examples of antibody fragments include linear antibody, single-chain antibody molecules and multispecific antibodies formed from antibody fragments.

The term “diabodies” refers to small antibody fragments prepared by constructing sFv fragments (see preceding paragraph) with short linkers (about 5-10) residues) between the V_{H} and V_{L} domains such that inter-chain but not intra-chain pairing of the variable domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains.

As used herein, a “chimeric antibody” refers to an antibody (immunoglobulin), such as a chimeric anti-TMEM106B antibody of the present disclosure, in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity. Chimeric antibodies of interest herein include PRIMATIZED® antibodies wherein the antigen-binding region of the antibody is derived from an antibody produced by, e.g., immunizing macaque monkeys with an antigen of interest. As used herein, “humanized antibody” is used a subset of “chimeric antibodies.”

“Humanized” forms of non-human (e.g., murine) antibodies, such as humanized forms of anti-TMEM106B antibodies of the present disclosure, are chimeric antibodies comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

A “human antibody” is one that possesses an amino-acid sequence corresponding to that of an antibody, such as an anti-TMEM106B antibody of the present disclosure, produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries and yeast-display libraries. Human antibodies can be prepared by administering the antigen to a transgenic animal that has been modified to produce such antibodies in response to antigenic challenge, but whose endogenous loci have been disabled, e.g., immunized xenomice as well as generated via a human B-cell hybridoma technology.

The term “hypervariable region,” “HVR,” or “HV,” when used herein refers to the regions of an antibody-variable domain, such as that of an anti-TMEM106B antibody of the present disclosure, that are hypervariable in sequence and/or form structurally defined loops. Generally, antibodies comprise six HVRs; three in the V_{H} (H1, H2, H3), and three in the V_{L} (L1, L2, L3). In native antibodies, H3 and L3 display the most diversity of the six HVRs, and H3 in particular is believed to play a unique role in conferring fine specificity to antibodies. Naturally occurring camelid antibodies consisting of a heavy chain only are functional and stable in the absence of light chain.

A number of HVR delineations are in use and are encompassed herein. In some embodiments, the HVRs may be Kabat complementarity-determining regions (CDRs) based on sequence variability and are the most commonly used (Kabat et al., supra). In some embodiments, the HVRs may be Chothia CDRs. Chothia refers instead to the location of the structural loops (Chothia and Lesk J. Mol. Biol. 196:901-917 (1987)). In some embodiments, the HVRs may be AbM HVRs. The AbM HVRs represent a compromise between the Kabat CDRs and Chothia structural loops, and are used by Oxford Molecular's AbM antibody-modeling software. In some embodiments, the HVRs may be “contact” HVRs. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat numbering) |
| H1 | H31-H35 | H26-H35 | H26-H32 | H30-H35 |
| | | | | (Chothia numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2), and 89-97 or 89-96 (L3) in the VL, and 26-35 (H1), 50-65 or 49-65 (a preferred embodiment) (H2), and 93-102, 94-102, or 95-102 (H3) in the VH. The variable-domain residues are numbered according to Kabat et al., supra, for each of these extended-HVR definitions.

“Framework” or “FR” residues are those variable-domain residues other than the HVR residues as herein defined.

An “acceptor human framework” as used herein is a framework comprising the amino acid sequence of a V_{L} or V_{H} framework derived from a human immunoglobulin framework or a human consensus framework. An acceptor human framework “derived from” a human immunoglobulin framework or a human consensus framework may comprise the same amino acid sequence thereof, or it may comprise pre-existing amino acid sequence changes. In some embodiments, the number of pre-existing amino acid changes are 10 or less, 9 or less, 8 or less, 7 or less, 6 or less, 5 or less, 4 or less, 3 or less, or 2 or less. Where pre-existing amino acid changes are present in a VH, preferable those changes occur at only three, two, or one of positions 71H, 73H and 78H; for instance, the amino acid residues at those positions may by 71A, 73T and/or 78A. In one embodiment, the VL acceptor human framework is identical in sequence to the V_{L} human immunoglobulin framework sequence or human consensus framework sequence.

A “human consensus framework” is a framework that represents the most commonly occurring amino acid residues in a selection of human immunoglobulin V_{L} or V_{H} framework sequences. Generally, the selection of human immunoglobulin V_{L} or V_{H} sequences is from a subgroup of variable domain sequences. Generally, the subgroup of sequences is a subgroup as in Kabat et al., Sequences of Proteins of Immunological Interest, 5th Ed. Public Health Service, National Institutes of Health, Bethesda, MD (1991). Examples include for the V_{L}, the subgroup may be subgroup kappa I, kappa II, kappa III or kappa IV as in Kabat et al., supra. Additionally, for the V_{H}, the subgroup may be subgroup I, subgroup II, or subgroup III as in Kabat et al., supra.

An “amino-acid modification” at a specified position, e.g., of an anti-TMEM106B antibody of the present disclosure, refers to the substitution or deletion of the specified residue, or the insertion of at least one amino acid residue adjacent the specified residue. Insertion “adjacent” to a specified residue means insertion within one to two residues thereof. The insertion may be N-terminal or C-terminal to the specified residue. The preferred amino acid modification herein is a substitution.

An “affinity-matured” antibody, such as an affinity matured anti-TMEM106B antibody of the present disclosure, is one with one or more alterations in one or more HVRs thereof that result in an improvement in the affinity of the antibody for antigen, compared to a parent antibody that does not possess those alteration(s). In one embodiment, an affinity-matured antibody has nanomolar or even picomolar affinities for the target antigen. Affinity-matured antibodies are produced by procedures known in the art. For example, Marks et al. Bio/Technology 10:779-783 (1992) describes affinity maturation by V_{H}- and V_{L}-domain shuffling. Random mutagenesis of HVR and/or framework residues is described by, for example: Barbas et al. Proc Nat. Acad. Sci. USA 91:3809-3813 (1994); Schier et al. Gene 169:147-155 (1995); Yelton et al. J. Immunol. 155: 1994-2004 (1995); Jackson et al. J. Immunol. 154(7):3310-9 (1995); and Hawkins et al., J. Mol. Biol. 226:889-896 (1992).

“Fv” is the minimum antibody fragment which comprises a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the VH and VL domains, which enables the sFv to form the desired structure for antigen binding.

Antibody “effector functions” refer to those biological activities attributable to the Fc region (a native sequence Fc region or amino acid sequence variant Fc region) of an antibody, and vary with the antibody isotype.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the present disclosure include human IgG1, IgG2, IgG3 and IgG4.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγU, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (“ITAM”) in its cytoplasmic domain. Inhibiting receptor FcγRIIB contains an immunoreceptor tyrosine-based inhibition motif (“ITIM”) in its cytoplasmic domain. Other FcRs, including those to be identified in the future, are encompassed by the term “FcR” herein. FcRs can also increase the serum half-life of antibodies.

As used herein, “percent (%) amino acid sequence identity” and “homology” with respect to a peptide, polypeptide or antibody sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms known in the art needed to achieve maximal alignment over the full-length of the sequences being compared.

The term “compete” when used in the context of antibodies (e.g., neutralizing antibodies) that compete for the same epitope means competition between antibody as determined by an assay in which the antibody being tested prevents or inhibits (e.g., reduces) specific binding of a reference molecule (e.g., a ligand, or a reference antibody) to a common antigen (e.g., TMEM106B or a fragment thereof). Numerous types of competitive binding assays can be used to determine if antibody competes with another, for example: solid phase direct or indirect radioimmunoassay (RIA), solid phase direct or indirect enzyme immunoassay (EIA), sandwich competition assay (see. e.g., Stahli et al., 1983, Methods in Enzymology 9:242-253); solid phase direct biotin-avidin EIA (see, e.g., Kirkland et al., 1986, J. Immunol. 137:3614-3619) solid phase direct labeled assay, solid phase direct labeled sandwich assay (see, e.g., Harlow and Lane, 1988, Antibodies, A Laboratory Manual, Cold Spring Harbor Press); solid phase direct label RIA using 1-125 label (see, e.g., Morel et al., 1988, Molec. Immunol. 25:7-15); solid phase direct biotin-avidin EIA (see, e.g., Cheung, et al., 1990, Virology 176:546-552); and direct labeled RIA (Moldenhauer et al., 1990, Scand. J. Immunol. 32:77-82). Typically, such an assay involves the use of purified antigen bound to a solid surface or cells bearing either of these, an unlabelled test antibody and a labeled reference antibody. Competitive inhibition is measured by determining the amount of label bound to the solid surface or cells in the presence of the test antibody. Usually the test antibody is present in excess. Antibodies identified by competition assay (competing antibodies) include antibodies binding to the same epitope as the reference antibody and antibodies binding to an adjacent epitope sufficiently proximal to the epitope bound by the reference antibody for steric hindrance to occur. Additional details regarding methods for determining competitive binding are provided below and, in the examples, herein. Usually, when a competing antibody is present in excess, it will inhibit (e.g., reduce) specific binding of a reference antibody to a common antigen by at least 20%, 30%, 40%, 50%, 60%, 70%, 80%, 85%, 90%, 95%, 97.5%, and/or near 100%.

As used herein, an “interaction” between a TMEM106B polypeptide and a second polypeptide encompasses, without limitation, protein-protein interaction, a physical interaction, a chemical interaction, binding, covalent binding, and ionic binding. As used herein, an antibody “inhibits interaction” between two polypeptides when the antibody disrupts, reduces, or completely eliminates an interaction between the two polypeptides. An antibody of the present disclosure, thereof, “inhibits interaction” between two polypeptides when the antibody thereof binds to one of the two polypeptides. In some embodiments, the interaction can be inhibited by at least about any of 20%, 30%, 40%, 50%, 60%, 70%, 80%, 85%, 90%, 95%, 97.5%, and/or near 100%.

The term “epitope” includes any determinant capable of being bound by an antibody. An epitope is a region of an antigen that is bound by an antibody that targets that antigen, and when the antigen is a polypeptide, includes specific amino acids that directly contact the antibody. Most often, epitopes reside on polypeptides, but in some instances, can reside on other kinds of molecules, such as nucleic acids. Epitope determinants can include chemically active surface groupings of molecules such as amino acids, sugar side chains, phosphoryl or sulfonyl groups, and can have specific three-dimensional structural characteristics, and/or specific charge characteristics. Generally, antibodies specific for a particular target antigen will preferentially recognize an epitope on the target antigen in a complex mixture of polypeptides and/or macromolecules.

An “agonist” antibody or an “activating” antibody is an antibody that induces (e.g., increases) one or more activities or functions of the antigen after the antibody binds the antigen.

An “antagonist” antibody or a “blocking” antibody or an “inhibitory” antibody is an antibody that reduces, inhibits, and/or eliminates (e.g., decreases) antigen binding to one or more ligand after the antibody binds the antigen, and/or that reduces, inhibits, and/or eliminates (e.g., decreases) one or more activities or functions of the antigen after the antibody binds the antigen. In some embodiments, antagonist antibodies, or blocking antibodies, or inhibitory antibodies substantially or completely inhibit antigen binding to one or more ligand and/or one or more activities or functions of the antigen.

An “isolated” antibody, such as an isolated anti-TMEM106B antibody of the present disclosure, is one that has been identified, separated and/or recovered from a component of its production environment (e.g., naturally or recombinantly). Preferably, the isolated antibody is free of association with all other contaminant components from its production environment. Contaminant components from its production environment, such as those resulting from recombinant transfected cells, are materials that would typically interfere with research, diagnostic or therapeutic uses for the antibody, and may include enzymes, hormones, and other proteinaceous or non-proteinaceous solutes. In preferred embodiments, the antibody will be purified: (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or, preferably, silver stain. Isolated antibody includes the antibody in situ within recombinant T-cells since at least one component of the antibody's natural environment will not be present. Ordinarily, however, an isolated polypeptide or antibody will be prepared by at least one purification step.

An “isolated” nucleic acid molecule encoding an antibody, such as an anti-TMEM106B antibody of the present disclosure, is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies herein is in a form other than in the form or setting in which it is found in nature. Isolated nucleic acid molecules therefore are distinguished from nucleic acid encoding the polypeptides and antibodies herein existing naturally in cells.

The term “vector,” as used herein, is intended to refer to a nucleic acid molecule capable of transporting another nucleic acid to which it has been linked. One type of vector is a “plasmid,” which refers to a circular double stranded DNA into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors,” or simply, “expression vectors.” In general, expression vectors of utility in recombinant DNA techniques are often in the form of plasmids. In the present specification, “plasmid” and “vector” may be used interchangeably as the plasmid is the most commonly used form of vector.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

“Carriers” as used herein include pharmaceutically acceptable carriers, excipients, or stabilizers that are nontoxic to the cell or mammal being exposed thereto at the dosages and concentrations employed.

As used herein, the term “preventing” includes providing prophylaxis with respect to occurrence or recurrence of a particular disease, disorder, or condition in an individual. An individual may be predisposed to, susceptible to a particular disease, disorder, or condition, or at risk of developing such a disease, disorder, or condition, but has not yet been diagnosed with the disease, disorder, or condition.

As used herein, an individual “at risk” of developing a particular disease, disorder, or condition may or may not have detectable disease or symptoms of disease, and may or may not have displayed detectable disease or symptoms of disease prior to the treatment methods described herein. “At risk” denotes that an individual has one or more risk factors, which are measurable parameters that correlate with development of a particular disease, disorder, or condition, as known in the art. An individual having one or more of these risk factors has a higher probability of developing a particular disease, disorder, or condition than an individual without one or more of these risk factors.

As used herein, the term “treatment” refers to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated”, for example, if one or more symptoms associated with a particular disease, disorder, or condition are mitigated or eliminated.

An “effective amount” refers to at least an amount effective, at dosages and for periods of time necessary, to achieve the desired therapeutic or prophylactic result. An effective amount can be provided in one or more administrations. An effective amount herein may vary according to factors such as the disease state, age, sex, and weight of the individual, and the ability of the treatment to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. An effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

An “individual” for purposes of treatment, prevention, or reduction of risk refers to any animal classified as a mammal, including humans, domestic and farm animals, and zoo, sport, or pet animals, such as dogs, horses, rabbits, cattle, pigs, hamsters, gerbils, mice, ferrets, rats, cats, and the like. In some embodiments, the individual is human.

As used herein, administration “in conjunction” with another compound or composition includes simultaneous administration and/or administration at different times. Administration in conjunction also encompasses administration as a co-formulation or administration as separate compositions, including at different dosing frequencies or intervals, and using the same route of administration or different routes of administration. In some embodiments, administration in conjunction is administration as a part of the same treatment regimen.

The term “about” as used herein refers to the usual error range for the respective value readily known to the skilled person in this technical field. Reference to “about” a value or parameter herein includes (and describes) embodiments that are directed to that value or parameter per se.

As used herein and in the appended claims, the singular forms “a,” “an,” and “the” include plural reference unless the context clearly indicates otherwise. For example, reference to an “antibody” is a reference to from one to many antibodies, such as molar amounts, and includes equivalents thereof known to those skilled in the art, and so forth.

As used herein a “TMEM106B” protein of the present disclosure includes, without limitation, a mammalian TMEM106B protein, human TMEM106B protein, primate TMEM106B protein, cynomolgus (cyno) TMEM106B protein, mouse TMEM106B protein, and rat TMEM106B protein. Additionally, anti-TMEM106B antibodies of the present disclosure may bind an epitope within one or more of a mammalian TMEM106B protein, human TMEM106B protein, primate TMEM106B, cyno TMEM106B protein, mouse TMEM106B protein, and rat TMEM106B protein.

It is understood that aspect and embodiments of the present disclosure described herein include “comprising,” “consisting,” and “consisting essentially of” aspects and embodiments.

### TMEM106B Protein

Provided herein are antibodies directed against TMEM106B protein. Antibodies provided are useful, e.g., for the diagnosis or treatment of TMEM106B associated disorders.

In one aspect, the present disclosure provides isolated (e.g., monoclonal) antibodies that bind to an epitope within a TMEM106B protein of the present disclosure. TMEM106B proteins of the present disclosure include, without limitation, a mammalian TMEM106B protein, human TMEM106B protein, mouse TMEM106B protein, and cynomolgus TMEM106B protein.

Human TMEM106B is a 274-amino acid protein that encodes a type 2 membrane glycoprotein. The amino acid sequence of human TMEM106B is set forth in SEQ ID NO:1:

MGKSLSHLPLHSSKEDAYDGVTSENMRNGLVNSEVHNEDGRNGDV
SQFPYVEFTGRDSVTCPTCQGTGRIPRGQENQLVALIPYSDQRLR
PRRTKLYVMASVFVCLLLSGLAVFFLFPRSIDVKYIGVKSAYVSY
DVQKRTIYLNITNTLNITNNNYYSVEVENITAQVQFSKTVIGKAR
LNNITIIGPLDMKQIDYTVPTVIAEEMSYMYDFCTLISIKVHNIV
LMMQVTVTTTYFGHSEQISQERYQYVDCGRNTTYQLGQSEYLNVL
QPQQ

Additionally, the amino acid sequence of mouse TMEM106B is set forth in SEQ ID NO:2:

GKSLSHLPLHSNKEDGYDGVTSTDNMRNGLVSSEVHNEDGRNGDV
SQFPYVEFTGRDSVTCPTCQGTGRIPRGQENQLVALIPYSDQRLR
PRRTKLYVMASVFVCLLLSGLAVFFLFPRSIEVKYIGVKSAYVSY
DAEKRTIYLNITNTLNITNNNYYSVEVENITAQVQFSKTVIGKAR
LNNITNIGPLDMK QIDYTVPTVIAEEMSYMYDFCTLLSIKVHNI
VLMMQVTVTTAYFGHSEQISQERYQYVDCGRNTTYQLAQSEYLNV
LQPQQ

Additionally, the amino acid sequence of cynomolgus (cyno) TMEM106B is set forth in SEQ ID NO:3:

MGKSLSHLPLHSSKEDAYDGVTSENMRNGLVNSEVHNEDGRNGDV
SQFPYVEFTGRDSVTRPTCQGTGRIPRGQENQLVALIPYSDQRLR
PRRTKLYVMASVFVCLLLSGLAVFFLFPRSIDVKYIGVKSAYVSY
DVQKRTIYLNITNTLNITNNNYYSVEVENITAQVQFAKTVIGKAR
LNNITHIGPLDMKQIDYTVPTVIAEEMSYMYDFCTLISIKVHNIV
LMMQVTVTTTYFGHSEQISQERYQYVDCGRNTTYQLGQSEYLNVL
QPQQ

In some embodiments, TMEM106B is expressed in a cell. In some embodiments, TMEM106B is expressed in endosomes and/or lysosomes. In some embodiments, TMEM106B is expressed in late endosomes and/or late lysosomes. In some embodiments, TMEM106B is expressed on the cell surface.

TMEM106B proteins of the present disclosure include several domains, including without limitation, an N-terminal lumenal domain (predicted to range from amino acid residues 11-274 of human TMEM106B; see SEQ ID NO:1), a transmembrane domain (predicted to range from amino acid residues 96-117 of human TMEM106B)), and a C-terminal domain (predicted to range from amino acid residues 1-92 of human TMEM106B). Additionally, TMEM106B proteins of the present disclosure are expressed in a number of tissues and cells, including without limitation, the brain, neurons, glial cells, endothelial cells, perivascular cells, pericytes, etc.

### TMEM106B Binding Partners

TMEM106B proteins of the present disclosure can interact with (e.g., bind to) one or more ligands or binding proteins, including, without limitation, progranulin protein (GRN), other TMEM106 protein family members, such as TMEM106B and TMEM106C, clathrin heavy chain (CLTC), the μ1 subunit of adipocyte protein 2 (AP2M1), charged multi-vesicular body protein 2b (CHMP2B), microtube-associated protein 6 (MAP6), lysosomal-associated membrane protein 1 (LAMP1), and vacuolar-ATPase accessory protein 1. Anti-TMEM106B antibodies of the present disclosure affect the interaction of TMEM106B with its various ligands and binding partners.

### Progranulin

TMEM106B has been shown to colocalize with progranulin in neuronal late endo-lysosomes, and TMEM106B overexpression increases intracellular levels of progranulin (Chen-Plotkin et al, 2012, J Neurosci, 32:11213-11227).

Progranulin is variously referred to as PGRN, proepithelin, granulin-epithelin precursor, PC (prostate cancer) cell-derived growth factor (PCDGF), and acrogranin. Progranulin is a 593-amino acid protein that encodes a 68.5 kD a secreted glycoprotein that has 7.5 repeats of smaller granulin (epithelin) motifs, ranging from 6-25 kDa, which can be proteolytically cleaved from the precursor PGRN. Examples of Progranulin cleavage products include, without limitation, granulin A/Epithelins 1, granulin B Epithelins 2, granulin C, granulins D, granulin E, granulin F, granulin G and any other known peptide products derived from Progranulin.

Progranulin is widely expressed, and in non-neuronal cells has been associated with a variety of events, such as cell cycle regulation and cell motility, wound repair, inflammation, induction of growth factors such as vascular endothelial growth factor (VEGF), and tumorigenesis. Progranulin is also widely expressed in early neural development, but becomes restricted in later development to defined neuronal populations, such as cortical neurons, hippocampal pyramidal neurons, and Purkinje cells.

Accordingly, in some embodiments, an anti-TMEM106B antibody of the present disclosure inhibits (e.g., blocks) or reduces the interaction between TMEM106B and progranulin. Alternatively, in some embodiments, an anti-TMEM106B antibody of the present disclosure enhances or increases the interaction between TMEM106B and progranulin.

### Other TMEM106 Family Members

TMEM106B proteins have been shown to interact with other TMEM106 protein family members. For example, the N-terminus of TMEM106B has been shown to interact with its family member TMEM106C. (See Stagi et al., 2014, Mol Cell Neurosci, 61:226-240.) TMEM106B proteins of the present disclosure bind to and modify the function and activity of TMEM106B. Additionally, TMEM106B proteins bind to and modify the function and activity of TMEM106C.

Accordingly, in some embodiments, an anti-TMEM106B antibody of the present disclosure inhibits (e.g., blocks) or reduces the interaction between TMEM106B and other TMEM106 protein family members. In some embodiments, an anti-TMEM106B antibody of the present disclosure inhibits (e.g., blocks) or reduces the interaction between TMEM106B and another TMEM106B polypeptide. In some embodiments, an anti-TMEM106B antibody of the present disclosure inhibits (e.g., blocks) or reduces the interaction between TMEM106B and TMEM106C. Alternatively, in some embodiments, an anti-TMEM106B antibody of the present disclosure enhances or increases the interaction between TMEM106B and other TMEM106 protein family members. In some embodiments, an anti-TMEM106B antibody of the present disclosure enhances or increases the interaction between TMEM106B and another TMEM106B polypeptide. In some embodiments, an anti-TMEM106B antibody of the present disclosure enhances or increases the interaction between TMEM106B and TMEM106C.

### Clathrin Heavy Chain

The N-terminus of TMEM106B has been shown to interact with the endocytic adaptor protein clathrin heavy chain (CLTC). (See Stagi et al., 2014, Mol Cell Neurosci, 61:226-240.) The protein interactions together with TMEM106B's endolysosomal localization imply that the TMEM106B cytoplasmic domain may participate in the delivery of endocytic cargos to the lysosome.

Accordingly, in some embodiments, an anti-TMEM106B antibody of the present disclosure inhibits (e.g., blocks) or reduces the interaction between TMEM106B and CLTC. Alternatively, in some embodiments, an anti-TMEM106B antibody of the present disclosure enhances or increases the interaction between TMEM106B and CLTC.

### μ1 Subunit of Adipocyte Protein 2

The N-terminus of TMEM106B has been shown to interact with the endocytic adaptor proteins μ1 subunit of adipocyte protein 2 (AP2M1). (See Stagi et al., 2014, Mol Cell Neurosci, 61:226-240.) The protein interactions together with TMEM106's endolysosomal localization imply that the TMEM106B cytoplasmic domain may participate in the delivery of endocytic cargos to the lysosome.

Accordingly, in some embodiments, an anti-TMEM106B antibody of the present disclosure inhibits (e.g., blocks) or reduces the interaction between TMEM106B and the μ1 subunit of adipocyte protein 2 (AP2M1). Alternatively, in some embodiments, an anti-TMEM106B antibody of the present disclosure enhances or increases the interaction between TMEM106B and the μ1 subunit of adipocyte protein 2 (AP2M1).

### Charged Multi-Vesicular Body Protein 2b

TMEM106B has been shown to directly bind charged multi-vesicular body protein 2b (CHMP2B), a member of the endosomal sorting complexes required for transport III (ESCRT-III) complex that regulates endolysosomal protein trafficking and autophagic structure formation (Jun et al., 2015, Mol Brain, 8:85). Accordingly, in some embodiments, an anti-TMEM106B antibody of the present disclosure inhibits (e.g., blocks) or reduces the interaction between TMEM106B and CHMP2B. Alternatively, in some embodiments, an anti-TMEM106B antibody of the present disclosure enhances or increases the interaction between TMEM106B and CHMP2B.

### Microtubule-Associated Protein 6

The C-terminus of microtubule-associated protein 6 (MAP6) directly binds the N-terminus of TMEM106B (Schwenk et al., 2014, EMBO J, 33:450-467). MAP6 overexpression inhibits dendritic branching similar to TMEM106B knockdown. MAP6 knockdown fully rescues the dendritic phenotype of TMEM106B knockdown, supporting a functional interaction between TMEM106B and MAP6. TMEM106B/MAP6 interaction was shown to be crucial for regulating dendritic trafficking of lysosomes, presumably by acting as a molecular break for retrograde transport. Lysosomal misrouting may promote neurodegeneration in patients with TMEM106B risk variants. The C-terminal repeat region of the neuron-enriched splice variant of MAP6 binds to the cytoplasmic N-terminus of TMEM106B preferentially.

Accordingly, in some embodiments, an anti-TMEM106B antibody of the present disclosure inhibits (e.g., blocks) or reduces the interaction between TMEM106B and MAP6. Alternatively, in some embodiments, an anti-TMEM106B antibody of the present disclosure enhances or increases the interaction between TMEM106B and MAP6.

### Lysosomal-Associated Membrane Protein 1

TMEM106B colocalizes with LAMP1 in late-endosomal/lysosomal vesicles in the cell body and in dendrites, but not with synaptic vesicles or early or recycling endosomes. (Stagi et al., 2014, Mol Cell Neurosci, 61:226-240.) LAMP1 (lysosomal-associated membrane protein 1) is a late endosomal/lysosomal marker. Reduction of TMEM106B increases axonally-transported lysosomes (increases motility), while TMEM106B elevation inhibits such transport and yields large lysosomes. Accordingly, in some embodiments, an anti-TMEM106B antibody of the present disclosure inhibits (e.g., blocks) or reduces the interaction between TMEM106B and LAMP1. In some embodiments, an anti-TMEM106B antibody of the present disclosure increases axonally-transported lysosomes. In some embodiments, an anti-TMEM106B antibody of the present disclosure increases motility of axonally-transported lysosomes. Alternatively, in some embodiments, an anti-TMEM106B antibody of the present disclosure enhances or increases the interaction between TMEM106B and LAMP1.

### Vacuolar-ATPase Subunit Accessory Protein 1

TMEM106B has also been shown to bind vacuolar-ATPase subunit accessory protein 1 (v-ATPase AP1) through its lumenal (C-terminal) domain (Klein et al., 2017, Neuron, 95:281-296). V-ATPase is responsible for lysosomal acidification; modulation of its function, such as by stabilization of the multi-unit protein complex, would affect lysosomal function. Accordingly, in some embodiments, an anti-TMEM106B antibody of the present disclosure inhibits (e.g., blocks) or reduces the interaction between TMEM106B and vacuolar-ATPase subunit accessory protein 1. Alternatively, in some embodiments, an anti-TMEM106B antibody of the present disclosure enhances or increases the interaction between TMEM106B and vacuolar-ATPase subunit accessory protein 1.

Further provided herein are methods of screening for anti-TMEM106B antibodies that bind TMEM106B, and that block the interactions between TMEM106B and one or more TMEM106B ligands or binding partners (e.g., Progranulin, other TMEM106 family members (i.e., TMEM106A, TMTM106C), Clathrin heavy chain, μ1 subunit of adipocyte protein 2, CHMP2B, microtubule-associated protein 6, lysosomal-associated membrane protein 1, and vacuolar-ATPase accessory protein 1). In some embodiments, a peptide library can be synthesized in which a TMEM106B protein is dissected into consecutive 15-mer and 25-mer peptides separated by one amino acid residue and subsequently spotted onto filters. Binding of a TMEM106B ligand or binding partner can then then tested for its ability to interact with the TMEM106B peptide or with peptides in the presence or absence of anti-TMEM106B antibodies by SPOT binding analysis (e.g., Frank, R and Overwin, H (1996) Methods. Mol. Biol. 66, 149-169; Reineke, U et al., (2002) J. Immunol. Methods 267, 13-26; and Andersen, O S et al., (2010) J, BIOLOGICAL CHEMISTRY 285, 12210-12222). In some embodiments, a cellulose support can be prepared as an N-modified cellulose-aminohydroxylpropyl ether membrane, and all rounds of synthesis are started with spot definition by 9-fluorenylmethoxycar-bonyalanine-pentafluoophenyl ester that creates an alanine linker between peptide and membrane. For example, an automated linear synthesis of stepwise addition of the different amino acids protected at their N-terminal by 9-fluorenyl-methoxycarbonyl and appropriate side-chain protection for the growing peptide chain. In some embodiments, the pattern of de-protection, activation, and coupling is continued until 16-mer peptides are produced, resulting in an equally distributed array of covalently anchored peptides to the cellulose support at their C-terminal ends with N-terminal free ends (Scham, D et al., (2000) J. Comb. Chem. 2, 361-369). In some embodiments, removal of the side protection group can be performed in two steps. First, the membrane can be treated with 90% trifluoroacetic acid (in dichlormethane, containing 3% triisobutylsilane and 2% H2O); and secondly with, for example, 60% trifluoroacetic acid (in dichlormethane, containing 3% triisobutylsilane and 2% H2O). To remove trifluoroacetic acid salts, the membrane can be washed several times with H2O, ethanol, Tris-buffered saline, and ethanol, and then dried. Finally, the membrane is blocked in blocking buffer dilated in Tris-buffered saline (pH 8.0) and supplemented with 5% sac-charose for 2 h before the predefined peptide library is ready for ligand binding analysis. In some embodiments, for binding studies of cellulose-bound peptides, membrane-bound libraries can be incubated with combined S-peptide and polyhistidine-tagged ligands in the presence or absence of anti-TMEM106B antibodies, for example, in blocking buffer overnight at 4° C., followed by a second incubation with 1 mg/ml of HRP-conjugated S-protein also in blocking buffer but for 3 h at room temperature. Subsequently, the membrane can be washed, for example, three times for 10 min with Tris-buffered saline before quantitative characterization of bound ligand may be carried out using the UptiLight chemiluminescence substrate and a LumiImager instrument, providing the spot signal intensities in Boehringer light units. Alternatively, detection of bound ligand can be performed by an immunochemical assay with an antibody against a histidine tag from and a secondary HRP-conjugated anti-mouse antibody. Incubations can be performed utilizing standard Western blotting procedures and spot detection.

Further provided herein are methods of screening for anti-TMEM106B antibodies that block interactions (e.g., binding) TMEM106B and one or more TMEM106B ligands or binding partners (e.g., Progranulin, other TMEM106 family members (i.e., TMEM106A, TMEM106C), Clathrin heavy chain, μ1 subunit of adipocyte protein 2, CHMP2B, microtubule-associated protein 6, lysosomal-associated membrane protein 1, and vacuolar-ATPase accessory protein 1).

In some embodiments, the interaction between TMEM106B and TMEM106B ligands or binding partners (e.g., Progranulin, other TMEM106 family members (i.e., TMEM106A, TMEM106C), Clathrin heavy chain, μ1 subunit of adipocyte protein 2, CHMP2B, microtubule-associated protein 6, lysosomal-associated membrane protein 1, and vacuolar-ATPase accessory protein 1) may be characterized using surface Plasmon resonance analysis (e.g., Skeldal et al., 2012 J Biol Chem., 287:43798; and Andersen et al., 201, J Biol Chem, 285, 12210-12222). Determination of direct binding of TMEM106B ligand or binding partner to immobilized TMEM106B in the presence or absence of blocking anti-TMEM106B antibodies can be performed, for example, on a Biacore2000 instrument (Biacore, Sweden) using CaHBS as standard running buffer (10 mM HEPES, pH 7.4, 140 mM NaCl, 2 mM CaCl₂), 1 mM EGTA, and 0.005% Tween 20). In some embodiments, a biosensor chip from Biacore (CM5) can be activated using the NHS/EDC method followed by coating with TMEM106B to a protein density of 79 fmol/mm2 and used for affinity measurements of the binding partner. Preparation of a biosensor surface with pro-TMEM106B will follow an equal procedure. Regeneration of the flow cell after each cycle of ligand binding experiment can be done by two 10-μl pulses of regeneration buffer (10 mM glycine-HCl, pH 4.0, 500 mM NaCl, 20 mM EDTA, and 0.005% Tween 20) and a single injection of 0.001% SDS. Fitting of sensorgrams for affinity estimations can be done, for example, by using BIAevaluation version 3.1. Following similar protocols, immobilization of HisS-NGFpro or HisS-BDNFpro may also done on a CM5 biosensor chip using the NHS/EDC coupling kit, giving similar surface densities of immobilized protein (˜300 fmol/mm2). A biosensor chip with immobilized with a TMEM106B ligand or binding partner can also be used to examine the binding of TMEM106B in the absence or presence of competing TMEM106B antibodies.

In some embodiments, the interaction between TMEM106B and TMEM106B ligands and binding partners (e.g., Progranulin, other TMEM106 family members (i.e., TMEM106A, TMEM106C), Clathrin heavy chain, μ1 subunit of adipocyte protein 2, CHMP2B, microtubule-associated protein 6, lysosomal-associated membrane protein 1, and vacuolar-ATPase accessory protein 1) can be characterized using a pulldown assay (e.g., Andersen et al., 2010, J Biol Chem, 285, 12210-12222). For example, expressed intracellular or extracellular domains of TMEM106B can be incubated with tagged TMEM106B ligands or binding partners in the absence or presence of TMEM106B blocking antibodies and are precipitated using 100 μl of glutathione (GSH)-Sepharose beads (Amersham Biosciences, catalog no. 17-0756-01). The amount of applied receptor domains can be determined by precipitation using Talon beads as control. Bound proteins can be separated by SDS-PAGE analysis and visualized using anti-histidine antibody by standard Western blotting analysis.

In some embodiments, the interaction between TMEM106B and TMEM106B ligands and binding partners (e.g., Progranulin, other TMEM106 family members (i.e., TMEM106A, TMEM106C), clathrin heavy chain, μ1 subunit of adipocyte protein 2, CHMP2B, microtubule-associated protein 6, lysosomal-associated membrane protein 1, and vacuolar-ATPase accessory protein 1) may be characterized using cellulose-bound proteins (e.g., Andersen et al., 2010, J Biol Chem, 285, 12210-12222). For example, membrane-bound proteins can be incubated with S-peptide and polyhistidine-tagged Progranulin, other TMEM106 family members (i.e., TMEM106A, TMEM106C), clathrin heavy chain, μ1 subunit of adipocyte protein 2, CHMP2B, microtubule-associated protein 6, lysosomal-associated membrane protein 1, vacuolar-ATPase accessory protein 1, or another TMEM106B ligand or binding partner; in blocking buffer overnight at 4° C., followed by a second incubation with 1 g/ml of HRP-conjugated S-protein also in blocking buffer but for 3 hours at room temperature. Subsequently, the membrane may be washed three times for 10 minutes with Tris-buffered saline before quantitative characterization of bound ligand is carried out using the UptiLight chemiluminescence substrate and a LumiImager instrument, providing the spot signal intensities in Boehringer light units. Alternatively, detection of bound ligand can be performed by an immunochemical assay with an antibody against the histidine tag and a secondary HRP-conjugated anti-mouse antibody. Incubations can be followed by standard Western blotting analysis and spot detection.

In some embodiments, the interaction between TMEM106B and TMEM106B ligands and binding partners (e.g., Progranulin, other TMEM106 family members (i.e., TMEM106A, TMEM106C), Clathrin heavy chain, μ1 subunit of adipocyte protein 2, CHMP2B, microtubule-associated protein 6, lysosomal-associated membrane protein 1, and vacuolar-ATPase accessory protein 1) may be characterized using a proximity ligation assay (e.g., Gustafsen et al., 2013 The Journal of Neuroscience, 33:64-71). For example, proximity ligation assay (PLA) (DuolinkII) on cells expressing or exposed to TMEM106B and its ligand or binding partner can be performed with the primary antibodies anti-TMEM106B, and antibodies against the binding partner, followed by incubation with secondary antibodies conjugated to oligonucleotides, which hybridize to subsequently added circle-forming oligonucleotides and prime a rolling circle amplification when the antigens are located within proximity of 40 nm. The amplified DNA can be visualized by addition of complementary fluorescent-labeled oligonucleotides.

In some embodiments, the interaction between TMEM106B and TMEM106B ligands and binding partners (e.g., Progranulin, other TMEM106 family members (i.e., TMEM106A, TMEM106C), Clathrin heavy chain, μ1 subunit of adipocyte protein 2, CHMP2B, microtubule-associated protein 6, lysosomal-associated membrane protein 1, and vacuolar-ATPase accessory protein 1) may be characterized using alkaline phosphatase-tagged ligands in cell binding assays (e.g., Hu et al., 2005, J. Neurosci. 25, 5298-5304; Fournier et al., 2001, Nature 409, 341-346; Lauren et al., 2009, Nature 457, 1128-1132; and Hu et al., 2010, Neuron 68, 654-667). For example, alkaline phosphatase (AP)-tagged ligands can be made to assess binding to TMEM106B on transfected cells or primary neurons. To detect AP tagged ligand binding to cells expressing TMEM106B, cultures can be washed with, for example, Hanks balanced salt solution containing 20 mM sodium HEPES, pH 7.05, and 1 mg/ml bovine serum albumin (BSA) (HBH). Then, the plates can be incubated with AP tagged ligands in the presence or absence of TMEM106B blocking antibodies, for example, in HBH for 2 h at 23° C. AP bound ligand can be detected and quantified according to methods well-known in the art.

In certain embodiments that may be combined with any of the embodiments provided herein, the anti-TMEM106B antibody further inhibits interaction between TMEM106B and one or more of its ligands, signaling proteins or binding proteins by: a) reducing the effective levels of TMEM106B available for interacting with the one or more ligands or binding proteins; b); blocking one or more of the sites on TMEM106B required for interaction with the one or more ligands or binding proteins; c) preventing one or more posttranslational events on TMEM106B that are required for interaction with the one or more ligands or binding proteins and/or for correct processing and/or subcellular localization of TMEM106B; d) inducing degradation of TMEM106B; e) changing the conformation of TMEM106B, or both. In certain embodiments that may be combined with any of the preceding embodiments, the anti-TMEM106B antibody binds specifically to human TMEM106B, mouse TMEM106B, cyno TMEM106B, or a combination thereof. In certain embodiments that may be combined with any of the preceding embodiments, the anti-TMEM106B antibody is a human antibody, a humanized antibody, a bispecific antibody, a monoclonal antibody, a multivalent antibody, a conjugated antibody, or a chimeric antibody. In certain embodiments that may be combined with any of the preceding embodiments, the anti-TMEM106B antibody is a bispecific antibody recognizing a first antigen and a second antigen. In certain embodiments that may be combined with any of the preceding embodiments, the first antigen is TMEM106B and the second antigen is an antigen facilitating transport across the blood-brain-barrier. In certain embodiments that may be combined with any of the preceding embodiments, the second antigen is selected from the group consisting of TMEM106B, transferrin receptor (TR), insulin receptor (HIR), insulin-like growth factor receptor (IGFR), low-density lipoprotein receptor related proteins 1 and 2 (LPR-1 and 2), diphtheria toxin receptor, CRM197, a llama single domain antibody, TMEM 30(A), a protein transduction domain, TAT, Syn-B, penetratin, a poly-arginine peptide, an angiopep peptide, basigin, Glut1, and CD98hc, and ANG1005.

### TMEM106B Proteolysis

TMEM106B undergoes intramembrane proteolysis and is processed to an N-terminal fragment (NTF) containing the transmembrane and intracellular domains; this processing is lysosomal protease-dependent. (Brady et al., 2014, J Biol Chem, 289:19670-19680) The GxGD aspartyl protease SPPL2A (and to a lesser extent SPPL2B) have been shown to be responsible for this intramembrane cleavage event. Additionally, it has been reported that an uncharacterized lysosomal protease(s) and SPPL2A cleave TMEM106B at around the 127th and 106th amino acids from the N-terminus, respectively, to generate two NTFs of TMEM106B (Brady et al., 2014, supra); however, the precise cleavage sites have not yet been identified. Overexpression of TMEM106B resulted in the appearance of NTF17 (1-127) and NTF13 (1-106), likely mediated by caspase activity.

Accordingly, in some embodiments, an anti-TMEM106B antibody of the present disclosure inhibits proteolysis of TMEM106B. The ability of an anti-TMEM106B antibody to inhibit proteolysis of TMEM106B can be determined by Western blotting of HEK293T cells overexpressing TMEM106B in the presence and absence of an anti-TMEM106B antibody. A decrease in the detection of proteolytic fragments of TMEM106B in the presence of the anti-TMEM106B antibody as compared to in the absence of the anti-TMEM106B antibody indicates that the antibody inhibits proteolysis of TMEM106B. In some embodiments, an anti-TMEM106B antibody of the present disclosure inhibits intramembrane proteolysis of TMEM106B. In some embodiments, an anti-TMEM106B antibody of the present disclosure inhibits proteolysis of TMEM106B, thereby preventing cleavage of TMEM106B into N-terminal fragments. In some embodiments, an anti-TMEM106B antibody of the present disclosure inhibits GxGD aspartyl protease SPPL2A cleavage of TMEM106B. In other embodiments, an anti-TMEM106B antibody of the present disclosure inhibits GxGD aspartyl protease SPPL2B cleavage of TMEM106B. In some embodiments, an anti-TMEM106B antibody of the present disclosure inhibits caspase-mediated cleavage of TMEM106B.

### TMEM106B as a Disease Target and Risk Factor in Various Neurodegenerative Disorders

Genome-wide association studies (GWAS) performed in 2509 control subjects and 515 subjects with pathologically confirmed FTLD-TDP, some of which contained a GRN mutation (van Deerlin et al., 2010, Nat Genetics, 42:234-239), identified TMEM106B as a genetic risk factor for FTD. This GWAS led to the discovery of 3 SNPs within a 68 kb region on chromosome 7p21.3 that associated with the occurrence of FTLD-TDP with genome-wide significance (rs6966915, rs102004, and top marker SNP rs1990622; p value range=5.00×10-11 to 1.08×10-11). The three significantly associated SNPs were in the same linkage disequilibrium (LD) block as nine other SNPs of nominal association, and all spanned the TMEM106B gene locus. In this case control cohort, the minor allele of each significant SNP was greatly underrepresented in FTLD-TDP patients (32.1 versus 43.6% in controls for rs1990622 minor C-allele; p value=1.08×10-11), suggesting that individuals expressing the minor TMEM106B alleles are less likely to develop disease [odds ratio (OR)=0.61 for the rs1990622 minor allele]. Upon stratification of the initial GWAS cohort by GRN mutation status, the association of the top three SNPs remained significant in both groups, but was greatest in people with GRN-related FTLD-TDP (rs1990622 p value=1.34×10-9; OR 0.34) in GRN carriers versus in non-GRN carriers (rs1990622 p value=6.90×10-7; OR 0.68). These SNPs conferred the strongest risk in patients also carrying a GRN mutation suggesting a functional interaction between TMEM106B and GRN. The genetic association of TMEM106B variants with FTLD-TDP was replicated with high confidence (Cruchaga et al., 2011; Finch et al., 2011; Van der Zee et al., 2011). In addition to increasing the risk of FTLD, the major allele at rs1990622, also reduces the age of FTLD onset. (See, e.g., Finch et al., 2011, Neurology, 76:467-474; Cruchaga et al., 2011, Arch Neurol.)

Accordingly, TMEM106B has been associated with various diseases, disorders, and conditions. Frontotemporal lobar degeneration (FTLD) (or frontotemporal dementia (FTD)) is the third most common neurodegenerative disease after Alzheimer's disease and Parkinson's disease and accounts for 20% of pre-senile dementia cases. (See, e.g., Rademakers et al., 2012, Nat Rev Neurol, 8:423-434). The condition results from the progressive deterioration of the frontal lobe of the brain. Over time, the degeneration may advance to the temporal lobe. The clinical presentation is diverse and the symptoms include dementia, behavioral changes, as well as speech and language impairment. (See, e.g., Cruts & Van Broeckhoven, 2008, Trends Genet. 24:186-194; Neary et al., 1998, Neurology 51:1546-1554; Ratnavalli, et al., 2002, Neurology 58:1615-1621.) Additional symptoms of upper or lower motoneuron disease are common and indicate a partial overlap with amyotrophic lateral sclerosis (ALS). The majority of FTLD cases show neuronal cytoplasmic aggregates of the nuclear DNA/RNA-binding protein TDP-43 (Neumann et al., 2006, Science, 314:130-133) and pathogenic mutations in TARDBP, the gene coding for TDP-43 are rare and predominantly cause ALS (Sreedharan et al., 2008, Science, 319:1668-1672). Familial forms of FTLD with TDP-43 pathology are mainly caused by hexanucleotide repeat expansion in C9orf72 (DeJesus-Hemandez et al., 2011; Renton et al., 2011) and dominant loss-of-function mutations in the growth factor progranulin (GRN) (Cruts et al., 2006, Curr Alzheimer Res, 3:485-491). While identification of the mutations associated with the rare familial forms of the disease yielded insight into FTLD pathogenesis, the etiology of the more common sporadic cases is more elusive, made further complex by a variability in clinical and neuropathologcial presentation.

A substantial portion of FTD cases are inherited in an autosomal dominant fashion, but even in one family, symptoms can span a spectrum from FTD with behavioral disturbances, to Primary Progressive Aphasia, to Cortico-Basal Ganglionic Degeneration. FTD, like most neurodegenerative diseases, can be characterized by the pathological presence of specific protein aggregates in the diseased brain. Historically, the first descriptions of FTD recognized the presence of intraneuronal accumulations of hyperphosphorylated Tau protein in neurofibrillary tangles or Pick bodies. A causal role for the microtubule associated protein Tau was supported by the identification of mutations in the gene encoding the Tau protein in several families (Hutton, M., et al., Nature 393:702-705 (1998). However, the majority of FTD brains show no accumulation of hyperphosphorylated Tau but do exhibit immunoreactivity to ubiquitin (Ub) and TAR DNA binding protein 43 (TDP-43) (Neumann et al., 2007, Arch. Neurol. 64:1388-1394). A majority of those FTD cases with Ub inclusions (FTD-U) were shown to carry mutations in the Progranulin gene.

Single nucleotide polymorphisms (SNPs) identified on chromosome 7 led to the discovery of the first genetic risk factors for FTLD-TDP as SNPs in the genomic region encoding TMEM106B. (Van Deerlin et al., 2010, Nat Genetics, 42:234-239.)

The initial studies assessing TMEM106B SNPs as disease risk factors in FTLD-TDP were conducted prior to the discovery of C9orf72 repeat expansion in 2012. Since this discovery, two independent groups found TMEM106B SNPs to also associate with one's risk of developing FTLD and/or ALS caused by C9orf72 mutations. (See Gallagher et al., 2014, Acta Neuropathol, 127:407-418; van Blitterswijk et al., 2014, Acta Neuropath, 127:397-406.) In these C9orf72 mutant cohorts, the frequency of individuals carrying the minor allele of TMEM106B SNPs [rs1990622 and/or rs3173615 in LD with rs1990622 was significantly reduced, although not as markedly as in cohorts of GRN mutation carriers. Further analysis of C9orf72 repeat expansion carriers in groups based on their predominant disease presentation as either FTLD, FTLD-ALS, or ALS, it was shown that TMEM106B SNPs specifically protect against the development of FTLD but not ALS, with less TDP-43 burden in the brains of C9orf72 expansion carriers homozygous for the protective TMEM106B alleles as compared to risk allele carriers. These findings are in agreement with an earlier examination of TMEM106B SNPs in a clinical cohort of ALS patients in which TMEM106B SNPs rs1990622 and rs1020004 were not associated with disease risk, but did significantly associate with cognitive function in ALS patients, with individuals homozygous for the rs1990622 minor allele having better cognitive performance than individuals heterozygous or homozygous for the major risk alleles (Vass et al., 2011, Acta Neuropathol, 121:373-380).

Neurodegenerative disease hallmarks, such as TDP-43 aggregates, are not specific to FTLD, and are observed in other neurodegenerative diseases, including Alzheimer's disease (AD), Lewy body dementia (LBD), and hippocampal sclerosis (HpScl) (Amador-Ortiz et al., 2007, Ann Neurol, 61:435-445; Zarow et al., 2008, Curr Neurol Neurosci Rep, 8:363-370), but also even in apparently healthy individuals, albeit to a limited extent (See, e.g., Yu et al., 2015, Neurology, 84:927-934). TMEM106B risk variants have been associated with TDP-43 neuropathology in the absence of a clinical neurological diagnosis. It was similarly found to affect the pathological presentation of AD with the protective TMEM106B haplotype associated with less TDP-43 pathology among AD patients (Rutherford et al., 2012, Neurology, 79:717-718). Hippocampal sclerosis is also common pathological hallmark in elderly patients with dementia, including FTLD-TDP and AD, often co-occurring with TDP-43 pathology. TMEM106B genotype was found to be associated with primary hippocampal sclerosis (Aoki et al., 2015, Acta Neuropathol, 129:53-64) as well as with hippocampal sclerosis pathology among AD patients, making TMEM106B as the strongest genetic indicator of AD-HpScl and HpScl known to date (Murray et al., 2014, Acta Neuropathol, 128:411-421). These studies collectively suggest that TMEM106B SNPs are risk factors for the development and severity of TDP-43 proteinopathy in non-FTLD disorders such as AD and HpScl.

A genomics study across over 1500 human brain autopsied samples identified common variants at TMEM106B as the main genome-wide determinant of the rate of biological aging in human brain: the presence of 2 risk alleles at the TMEM106B gene locus making an individual appear ˜12 years older based on its gene expression profile than his/her actual age (Rhinn and Abeliovich, 2017, Cell Syst, 4:404-415). This effect of TMEM106B risk alleles is seen in individuals free of known neurological disease as well as in individuals with neurodegenerative processes such as Alzheimer's. The role of TMEM106B in aging appeared highly selective, in terms of brain and life (cortex and not cerebellum, specifically over 65 yo). The effect was confirmed independently in additional cohorts in which carriers of the protective TMEM106B haplotype displayed reduced age-associated cognitive decline (Rhinn and Abeliovich, 2017, supra). The effect on cognitive performance was confirmed as the TMEM106B protective haplotype was shown to be associated with better cognitive performance for a given amount of cerebral pathology (White et al., 2017, PLoS Med, 14:e1002287). Those results strengthen the pleiotropic role of TMEM106B in aging beyond neurodegenerative diseases.

Evidence suggests that TMEM106B variants increase risk for developing FTLD-TDP by increasing TMEM106B mRNA and protein expression levels. Increased TMEM106B results in a decrease in the average number of late endosomes/lysosomes per cell, loss of lysosomal acidification, and impaired lysosomal degradation.

Frontotemporal lobar degeneration with TDP-43 inclusions (FTLD-TDP) is a fatal neurogenerative disease with no available treatments. Mutations in the progranulin gene (GRN) causing impaired production or secretion of progranulin are a common cause of FTLD-TDP. As discussed above, TMEM106B has been linked by genome-wide association to FTLD-TDP with and without GRN mutations. Increasing TMEM106B expression to model disease resulted in enlargement and poor acidification of endo-lysosomes, as well as impairment of mannose-6-phosphate-receptor trafficking. Endogenous neuronal TMEM106B colocalizes with progranulin in late endo-lysosomes, and TMEM106B overexpression increases intracellular levels of progranulin. (Chen-Plotkin et al., 2012, J Neurosci, 32:11213-11227.) In some embodiments, the present disclosure provides methods for preventing, reducing risk, or treating FTLD-TDP by administering to an individual in need thereof a therapeutically effective amount of an anti-TMEM-160B antibody of the present disclosure. In some embodiments, the present disclosure provides methods for

FTLD has been recognized as a disease that has a common pathologic background with amyotrophic lateral sclerosis (ALS). ALS is an incurable motor neuron degenerative disease, characterized by loss of both upper and lower motor neurons. Approximately 15% of FTLD patients develop motor neuron disease, and more than 15% of ALS patients have cognitive and behavioral impairments. An important pathological hallmark of FTLD and ALS is a cytoplasmic transactive response DNA-binding protein-43 (TDP-43) inclusion. TDP-43 is the major component of inclusions in ˜50\hat{ }% of FTLD patients (subtype FTLD-TDP) and the majority of ALS patients. A genome-wide associated study and cohort studies have identified three SNPs (re1990622, rs6966915, and rs1020004) in TMEM106B gene region as genetic risk modifiers for FTLD-TDP. The risk association is more prominent in FTLD-TDP cases with GRN and C9ORF72 mutations. Overexpression of TMEM106B induces cell death, enhances oxidative stress-induced cytotoxicity, and causes the cleavage of TDP-43, using cell-based models, suggesting that up-regulation of TMEM106B increases the risk of FTLD by directly causing neurotoxicity. (Suzuki and Matsuoka, 2016, J Biol Chem, 291:21448-21460.) Additionally, TMEM106B has been implicated in the development of cognitive impairment in ALS (Vass et al., 2011, Acta Neuropathol, 121:373-380).

Hippocampal sclerosis of aging (HS-Aging) is a common, high morbidity-associated neurodegenerative condition in the elderly, and is diagnosed neuropathologically when neuron loss and astrocytosis are observed in the hippocampal formation, and are not considered attributable to Alzheimer's disease-type plaques and tangles. It has a clinical course similar to Alzheimer's disease. HS-Aging is distinguished from other hippocampal sclerosis disorders by the presence of TDP-43 pathology and the absence of severe symptoms or clinical signs of frontotemporal dementia. HS-Aging is associated with SNP rs1990622 (TMEM106b), and other SNPs, as well as polymorphisms in TMEM106b, ABCC9 (not associated with FTLD), and GRN. (Nelson et al., 2015, J Neuropathol Exp Neurol, 74:75-84.) In some embodiments, the present disclosure provides methods for preventing, reducing risk, or treating hippocampal sclerosis of aging by administering to an individual in need thereof a therapeutically effective amount of an anti-TMEM-160B antibody of the present disclosure.

A recent murine genetic knockout of TMEM106B (Klein et al. 2017, Neuron 95, 281-296) showed an effect for TMEM106B in the granulin pathway. Specifically, a genetic knock-out of the TMEM106B protein was able to rescue some of the pathogenic phenotype associated with GRN knockout in a mouse model, including a partial rescue of levels of lysosomal proteins, and rescue of behavioral changes such as hyperactivity and dis-inhibition. The TMEM106B knockout itself was well tolerated in the mice, as has been reported in other studies. In addition, this work suggested a possible mechanism of action of TMEM106B by showing a direct interaction (through co-immunoprecipitation) with the v-ATPase subunit AP1. The v-ATPase complex plays an important role in lowering the pH of lysosomes and thus initiating protein degradation and recycling. Thus, its interaction with TMEM106B may cause some of the lysosomal phenotypes associated with TMEM106B overexpression, and conversely blocking this interaction (or merely reducing the level of TMEM106B present) may be able to ameliorate this phenotype.

The above and additional analyses identify an association TMEM106B and various diseases, disorders, and conditions, such as without limitation, frontotemporal lobar degeneration, frontotemporal dementia, frontotemporal dementia with progranulin mutations, frontotemporal dementia with C9orf72 mutations, frontotemporal lobar degeneration with TDP-43 inclusions, TDP-43 proteinopathy, hippocampal sclerosis, hippocampal sclerosis of aging, cognitive impairments associated with various disorders (including without limitation cognitive impairment in amyotrophic lateral sclerosis and chronic traumatic encephalopathy), and hypomyelinating disorder (including without limitation hypomyelinating leukodystrophy). Accordingly, the present disclosure provides therapies targeting TMEM106B, including anti-TMEM106B antibodies that specifically bind TMEM106B and affect its function.

Chronic traumatic encephalopathy (CTE) is a progressive neurodegenerative disease that has been pathologically diagnosed in individuals with a history of repeated head impacts. TMEM106B has been shown to be an important factor in the progression of CTE and specifically in CTE-related neuropathology and dementia. Specifically, carriers of the minor (G) allele at SNP rs3173615 (T185S coding mutation) were shown to have decreased levels of neuropathology including lower AT8-positive p-tau levels, lower CD68-positive cell density, and increased levels of PSD-95, a post-synaptic marker often used to investigate synaptic loss. The G-allele was also associated with a 60% decrease in odds of ante-mortem dementia. In both cases, effects of the G allele were additive, with the GG genoptype being the most protective. TMEM106B geneotype had no effect on risk of disease; rather, the effect is seen in the severity of the disease progression.

Microglia are the primary innate immune cells of the central nervous system (CNS). Microglia exist in resting or activated states depending on the inflammatory milieu, which differs in the healthy CNS and in various disease states. TGFbeta is an important factor in microglial development and function and is required for the maintenance of the microglia-specific homeostatic gene signature. Suppression of TGFbeta signaling and of the TGFbeta pathway is a common feature of microglia isolated from neurodegenerative disease models. Additionally, TGFbeta has been described as having a critical function in preventing microglia/macrophage-mediated CNS pathology and neurodegeneration (Butovsky and Weiner, 2018, Nature, 19:622-635; Lund et al, 2018, Nature Immunol, 19:425-441). Accordingly, therapies that directly or indirectly increase TGFbeta expression, signaling, and/or function would provide benefit in the treatment of neurodegenerative diseases and disorders, including amyotrophic lateral sclerosis, Alzheimer's disease, multiple sclerosis, Parkinson's disease, autism spectrum disorder, dementia, etc. In some embodiments, anti-TMEM106B antibodies of the present disclosure are effective at increasing TGFbeta levels in cells.

Dementia is a non-specific syndrome (i.e., a set of signs and symptoms) that presents as a serious loss of global cognitive ability in a previously unimpaired person, beyond what might be expected from normal ageing. Dementia may be static as the result of a unique global brain injury. Alternatively, dementia may be progressive, resulting in long-term decline due to damage or disease in the body. While dementia is much more common in the geriatric population, it can also occur before the age of 65. Cognitive areas affected by dementia include, without limitation, memory, attention span, language, and problem solving. Generally, symptoms must be present for at least six months to before an individual is diagnosed with dementia.

Exemplary forms of dementia include, without limitation, frontotemporal dementia, Alzheimer's disease, vascular dementia, semantic dementia, and dementia with Lewy bodies.

Without wishing to be bound by theory, it is believed that administering an anti-TMEM106B antibody of the present disclosure can prevent, reduce the risk, and/or treat dementia.

Alzheimer's disease (AD) is the most common form of dementia. There is no cure for the disease, which worsens as it progresses, and eventually leads to death. Most often, AD is diagnosed in people over 65 years of age. However, the less-prevalent early-onset Alzheimer's diseases can occur much earlier.

Common symptoms of Alzheimer's disease include, behavioral symptoms, such as difficulty in remembering recent events; cognitive symptoms, confusion, irritability and aggression, mood swings, trouble with language, and long-term memory loss. As the disease progresses bodily functions are lost, ultimately leading to death. Alzheimer's disease develops for an unknown and variable amount of time before becoming fully apparent, and it can progress undiagnosed for years.

Amyotrophic lateral sclerosis (ALS) or, motor neuron disease or, Lou Gehrig's disease are used interchangeably and refer to a debilitating disease with varied etiology characterized by rapidly progressive weakness, muscle atrophy and fasciculations, muscle spasticity, difficulty speaking (dysarthria), difficulty swallowing (dysphagia), and difficulty breathing (dyspnea).

TMEM106B has also been implicated in hypomyelinating leukodystrophies. (Simons et al., 2017, Brain.) Hypomyelinating leukodystrophies are a heterogeneous group of disorders with clinical presentation that includes early-onset nystagmus, ataxia, and spasticity. Brain hypomyelination in four patients with hypomyelinating leukodystrophy showed the same dominant mutation (Aps252Asn) in TMEM106B, suggesting an association of TMEM106B in hypomyelinating disorders, possibly due to the role TMEM106B plays in lysosomal function. Accordingly, in some embodiments, the present disclosure provides a method for treating a hypomyelinating disorder in an individual in need thereof, the method comprising administering to the individual a therapeutically effective amount of an anti-TMEM106B antibody of the present disclosure. In some embodiments, the present disclosure provides a method for treating a hypomyelinating leukodystrophy in an individual in need thereof, the method comprising administering to the individual a therapeutically effective amount of an anti-TMEM106B antibody of the present disclosure.

The methods provided herein find use in preventing, reducing risk, or treating an individual having a neurodegenerative disease, disorder, or condition. In some embodiments, the present invention provides a method for preventing, reducing risk, or treating an individual having a neurodegenerative disorder, the method comprising administering to an individual in need thereof a therapeutically effective amount of an anti-TMEM106B antibody.

In some embodiments, the present invention provides a method for preventing, reducing risk, or treating an individual having a disease, disorder, condition, or injury characterized by the presence of TDP-43 inclusions, the method comprising administering to an individual in need thereof a therapeutically effective amount of an anti-TMEM106B antibody.

In some embodiments, the present invention provides a method for preventing, reducing risk, or treating an individual having a TDP-43 proteinopathy, the method comprising administering to an individual in need thereof a therapeutically effective amount of an anti-TMEM106B antibody.

In some embodiments, the present invention provides a method for preventing, reducing risk, or treating an individual having a disease, disorder, condition, or injury characterized by the presence of inflammatory cell debris or protein aggregates, the method comprising administering to an individual in need thereof a therapeutically effective amount of an anti-TMEM106B antibody.

In some embodiments, the present invention provides a method for preventing, reducing risk, or treating an individual having a disease, disorder, condition, or injury characterized by the presence of abnormal circulating myeloid cells, the method comprising administering to an individual in need thereof a therapeutically effective amount of an anti-TMEM106B antibody.

In some embodiments, the present invention provides a method for preventing, reducing risk, or treating an individual having unhealthy aging, the method comprising administering to an individual in need thereof a therapeutically effective amount of an anti-TMEM106B antibody.

In some embodiments, the present invention provides a method for preventing, reducing risk, or treating an individual having frontotemporal lobar degeneration (FTLD), the method comprising administering to an individual in need thereof a therapeutically effective amount of an anti-TMEM106B antibody.

In some embodiments, the present invention provides a method for preventing, reducing risk, or treating an individual having frontotemporal dementia (FTD), the method comprising administering to an individual in need thereof a therapeutically effective amount of an anti-TMEM106B antibody.

In some embodiments, the present invention provides a method for preventing, reducing risk, or treating an individual having frontotemporal dementia with progranulin mutations, the method comprising administering to an individual in need thereof a therapeutically effective amount of an anti-TMEM106B antibody.

In some embodiments, the present invention provides a method for preventing, reducing risk, or treating an individual having frontotemporal dementia with C90rf72 mutations, the method comprising administering to an individual in need thereof a therapeutically effective amount of an anti-TMEM106B antibody.

In some embodiments, the present invention provides a method for preventing, reducing risk, or treating an individual having frontotemporal lobar degeneration with TDP-43 inclusions, the method comprising administering to an individual in need thereof a therapeutically effective amount of an anti-TMEM106B antibody.

In some embodiments, the present invention provides a method for preventing, reducing risk, or treating an individual having hippocampal sclerosis (HpScl), the method comprising administering to an individual in need thereof a therapeutically effective amount of an anti-TMEM106B antibody.

In some embodiments, the present invention provides a method for preventing, reducing risk, or treating an individual having hippocampal sclerosis of aging (HS-Aging), the method comprising administering to an individual in need thereof a therapeutically effective amount of an anti-TMEM106B antibody.

In some embodiments, the present invention provides a method for preventing, reducing risk, or treating an individual having Alzheimer's disease, the method comprising administering to an individual in need thereof a therapeutically effective amount of an anti-TMEM106B antibody.

In some embodiments, the present invention provides a method for preventing, reducing risk, or treating an individual having Lewy body dementia, the method comprising administering to an individual in need thereof a therapeutically effective amount of an anti-TMEM106B antibody.

In some embodiments, the present invention provides a method for preventing, reducing risk, or treating an individual having cognitive impairment, the method comprising administering to an individual in need thereof a therapeutically effective amount of an anti-TMEM106B antibody.

In some embodiments, the present invention provides a method for preventing, reducing risk, or treating an individual having age related cognitive impairment, the method comprising administering to an individual in need thereof a therapeutically effective amount of an anti-TMEM106B antibody.

In some embodiments, the present invention provides a method for preventing, reducing risk, or treating an individual having CTE-related cognitive impairment, the method comprising administering to an individual in need thereof a therapeutically effective amount of an anti-TMEM106B antibody.

In some embodiments, the present invention provides a method for preventing, reducing risk, or treating an individual having age related brain atrophy, the method comprising administering to an individual in need thereof a therapeutically effective amount of an anti-TMEM106B antibody.

In some embodiments, the present invention provides a method for preventing, reducing risk, or treating an individual having age-associated traits, including without limitations inflammation, neuronal loss, and cognitive deficits, such as cognitive defects in the absence of known brain disease, including cognitive deficits of the frontal cerebral cortex of older individuals, the method comprising administering to an individual in need thereof a therapeutically effective amount of an anti-TMEM106B antibody.

In some embodiments, the present invention provides a method for preventing, reducing risk, or treating an individual having cognitive impairment associated with amyotrophic lateral sclerosis, the method comprising administering to an individual in need thereof a therapeutically effective amount of an anti-TMEM106B antibody.

In some embodiments, the present invention provides a method for preventing, reducing risk, or treating an individual having a disease, disorder, or condition associated with over expression or increased activity of TMEM106B, the method comprising administering to an individual in need thereof a therapeutically effective amount of an anti-TMEM106B antibody.

In some embodiments, the present invention provides a method for preventing, reducing risk, or treating an individual having a hypomyelinating disorder, the method comprising administering to an individual in need thereof a therapeutically effective amount of an anti-TMEM106B antibody.

In some embodiments, the present invention provides a method for reducing or inhibiting metastasis in an individual in need thereof, the method comprising administering to an individual in need thereof a therapeutically effective amount of an anti-TMEM106B antibody.

In some embodiments, the present invention provides a method for preventing, reducing risk, or treating an individual having cancer, the method comprising administering to an individual in need thereof a therapeutically effective amount of an anti-TMEM106B antibody.

### Exemplary Anti-TMEM106B Antibodies and Certain Other Antibody Embodiments

In some embodiments, provided herein are anti-TMEM106B antibodies comprising at least one, two, three, four, five, or six HVRs selected from: (a) HVR-H1 comprising an amino acid sequence selected from the group consisting of SEQ ID NOs:77, 78, 79, 80, 81, 82, 83, 84, 85, 86, 87, 88, 89, 90, 91, 92, 93, 94, 95, 96, 97, 98, 99, 100, 101, 102, 103, 104, 105, and 106; (b) HVR-H2 comprising an amino acid sequence selected from the group consisting of SEQ ID NOs:107, 108, 109, 110, 111, 112, 113, 114,115, 116, 117, 118, 119, 120, 121, 122,123, 124, 125, 126, 127, 128,129, 130, 131, 132, 133, 134, 135, 136, 137, 138, 139, 140, 141, and 142; (c) HVR-H3 comprising an amino acid sequence selected from the group consisting of SEQ ID NOs:143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, and 175; (d) HVR-L1 comprising an amino acid sequence selected from the group consisting of SEQ ID NOs:176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, and 208; (e) HVR-L2 comprising an amino acid sequence selected from the group consisting of SEQ ID NOs:209, 210, 211, 212, 213, 214, 215, 216, 217, 218, 219, 220, 221, 222, 223, 224, 225, 226, 227, 228, 229, 230, and 231; and (f) HVR-L3 comprising an amino acid sequence selected from the group consisting of SEQ ID NOs:232, 233, 234, 235, 236, 237, 238, 239, 240, 241, 242, 243, 244,245, 246, 247, 248, 249, 250, 251, 252, 253, 254, 255, 256, 257, 258, 259, 260, 261, 262, and 263.

In some embodiments, provided herein are anti-TMEM106B antibodies comprising at least one, at least two, or all three V_{H} HVR sequences selected from (a) HVR-H1 comprising an amino acid sequence selected from the group consisting of SEQ ID NOs:77, 78, 79, 80, 81, 82, 83, 84, 85, 86, 87, 88, 89, 90, 91, 92, 93, 94, 95, 96, 97, 98, 99, 100, 101, 102, 103, 104, 105, and 106; (b) HVR-H2 comprising an amino acid sequence selected from the group consisting of SEQ ID NOs:107, 108, 109, 110, 111, 112, 113, 114,115, 116, 117, 118, 119, 120, 121, 122,123, 124, 125, 126, 127, 128,129, 130, 131, 132, 133, 134, 135, 136, 137, 138, 139, 140, 141, and 142; and (c) HVR-H3 comprising an amino acid sequence selected from the group consisting of SEQ ID NOs:143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, and 175.

In some embodiments, provided herein are anti-TMEM106B antibodies comprising at least one, at least two, or all three V_{L} HVR sequences selected from (a) HVR-L1 comprising an amino acid sequence selected from the group consisting of SEQ ID NOs:176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, and 208; (b) HVR-L2 comprising an amino acid sequence selected from the group consisting of SEQ ID NOs:209, 210, 211, 212, 213, 214, 215, 216, 217, 218, 219, 220, 221, 222, 223, 224, 225, 226, 227, 228, 229, 230, and 231; and (c) HVR-L3 comprising an amino acid sequence selected from the group consisting of SEQ ID NOs:232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, and 263.

In some embodiments, provided herein are anti-TMEM106B antibodies comprising (a) a V_{H} domain comprising at least one, at least two, or all three V_{H} HVR sequences selected from (i) HVR-H1 comprising an amino acid sequence selected from the group consisting of SEQ ID NOs:77, 78, 79, 80, 81, 82, 83, 84, 85, 86, 87, 88, 89, 90, 91, 92, 93, 94, 95, 96, 97, 98, 99, 100, 101, 102, 103, 104, 105, and 106, (ii) HVR-H2 comprising an amino acid sequence selected from the group consisting of SEQ ID NOs:107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, and 142, and (iii) HVR-H3 comprising an amino acid sequence selected from the group consisting of SEQ ID NOs:143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, and 175, and (b) a VL domain comprising at least one, at least two, or all three VL HVR sequences selected from (i) HVR-L1 comprising an amino acid sequence selected from the group consisting of SEQ ID NOs:176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, and 208, (ii) HVR-L2 comprising an amino acid sequence selected from the group consisting of SEQ ID NOs:209, 210, 211, 212, 213, 214, 215, 216, 217, 218, 219, 220, 221, 222, 223, 224, 225, 226, 227, 228, 229, 230, and 231, and (iii) HVR-L3 comprising an amino acid sequence selected from the group consisting of SEQ ID NOs:232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, and 263.

In some embodiments, provided herein are anti-TMEM106B antibodies comprising: (a) HVR-H1 comprising the amino acid sequence of SEQ ID NO:77; (b) HVR-H2 comprising the amino acid sequence of SEQ ID NO:107; (c) HVR-H3 comprising the amino acid sequence of SEQ ID NO:143; (d) HVR-L1 comprising the amino acid sequence of SEQ ID NO:176; (e) HVR-L2 comprising the amino acid sequence of SEQ ID NO:209; and (f) HVR-L3 comprising the amino acid sequence of SEQ ID NO:232; (a) HVR-H1 comprising the amino acid sequence of SEQ ID NO:78; (b) HVR-H2 comprising the amino acid sequence of SEQ ID NO:108; (c) HVR-H3 comprising the amino acid sequence of SEQ ID NO:144; (d) HVR-L1 comprising the amino acid sequence of SEQ ID NO:177; (e) HVR-L2 comprising the amino acid sequence of SEQ ID NO:210; and (f) HVR-L3 comprising the amino acid sequence of SEQ ID NO:233; (a) HVR-H1 comprising the amino acid sequence of SEQ ID NO:78; (b) HVR-H2 comprising the amino acid sequence of SEQ ID NO:109; (c) HVR-H3 comprising the amino acid sequence of SEQ ID NO:144; (d) HVR-L1 comprising the amino acid sequence of SEQ ID NO:178; (e) HVR-L2 comprising the amino acid sequence of SEQ ID NO:210; and (f) HVR-L3 comprising the amino acid sequence of SEQ ID NO:233; (a) HVR-H1 comprising the amino acid sequence of SEQ ID NO:79; (b) HVR-H2 comprising the amino acid sequence of SEQ ID NO:110; (c) HVR-H3 comprising the amino acid sequence of SEQ ID NO:145; (d) HVR-L1 comprising the amino acid sequence of SEQ ID NO:176; (e) HVR-L2 comprising the amino acid sequence of SEQ ID NO:209; and (f) HVR-L3 comprising the amino acid sequence of SEQ ID NO:234; (a) HVR-H1 comprising the amino acid sequence of SEQ ID NO:80; (b) HVR-H2 comprising the amino acid sequence of SEQ ID NO:111; (c) HVR-H3 comprising the amino acid sequence of SEQ ID NO:146; (d) HVR-L1 comprising the amino acid sequence of SEQ ID NO:179; (e) HVR-L2 comprising the amino acid sequence of SEQ ID NO:211; and (f) HVR-L3 comprising the amino acid sequence of SEQ ID NO:235; (a) HVR-H1 comprising the amino acid sequence of SEQ ID NO:81; (b) HVR-H2 comprising the amino acid sequence of SEQ ID NO:112; (c) HVR-H3 comprising the amino acid sequence of SEQ ID NO:147; (d) HVR-L1 comprising the amino acid sequence of SEQ ID NO:180; (e) HVR-L2 comprising the amino acid sequence of SEQ ID NO:212; and (f) HVR-L3 comprising the amino acid sequence of SEQ ID NO:236; (a) HVR-H1 comprising the amino acid sequence of SEQ ID NO:82; (b) HVR-H2 comprising the amino acid sequence of SEQ ID NO:113; (c) HVR-H3 comprising the amino acid sequence of SEQ ID NO:148; (d) HVR-L1 comprising the amino acid sequence of SEQ ID NO:181; (e) HVR-L2 comprising the amino acid sequence of SEQ ID NO:210; and (f) HVR-L3 comprising the amino acid sequence of SEQ ID NO:237; (a) HVR-H1 comprising the amino acid sequence of SEQ ID NO:78; (b) HVR-H2 comprising the amino acid sequence of SEQ ID NO:114; (c) HVR-H3 comprising the amino acid sequence of SEQ ID NO:149; (d) HVR-L1 comprising the amino acid sequence of SEQ ID NO:182; (e) HVR-L2 comprising the amino acid sequence of SEQ ID NO:210; and (f) HVR-L3 comprising the amino acid sequence of SEQ ID NO:238; (a) HVR-H1 comprising the amino acid sequence of SEQ ID NO:83; (b) HVR-H2 comprising the amino acid sequence of SEQ ID NO:115; (c) HVR-H3 comprising the amino acid sequence of SEQ ID NO:150; (d) HVR-L1 comprising the amino acid sequence of SEQ ID NO:183; (e) HVR-L2 comprising the amino acid sequence of SEQ ID NO:213; and (f) HVR-L3 comprising the amino acid sequence of SEQ ID NO:239; (a) HVR-H1 comprising the amino acid sequence of SEQ ID NO:84; (b) HVR-H2 comprising the amino acid sequence of SEQ ID NO:116; (c) HVR-H3 comprising the amino acid sequence of SEQ ID NO:151; (d) HVR-L1 comprising the amino acid sequence of SEQ ID NO:184; (e) HVR-L2 comprising the amino acid sequence of SEQ ID NO:214; and (f) HVR-L3 comprising the amino acid sequence of SEQ ID NO:240; (a) HVR-H1 comprising the amino acid sequence of SEQ ID NO:85; (b) HVR-H2 comprising the amino acid sequence of SEQ ID NO:117; (c) HVR-H3 comprising the amino acid sequence of SEQ ID NO:152; (d) HVR-L1 comprising the amino acid sequence of SEQ ID NO:185; (e) HVR-L2 comprising the amino acid sequence of SEQ ID NO:210; and (f) HVR-L3 comprising the amino acid sequence of SEQ ID NO:241; (a) HVR-H1 comprising the amino acid sequence of SEQ ID NO:86; (b) HVR-H2 comprising the amino acid sequence of SEQ ID NO:118; (c) HVR-H3 comprising the amino acid sequence of SEQ ID NO:153; (d) HVR-L1 comprising the amino acid sequence of SEQ ID NO:186; (e) HVR-L2 comprising the amino acid sequence of SEQ ID NO:209; and (f) HVR-L3 comprising the amino acid sequence of SEQ ID NO:242; (a) HVR-H1 comprising the amino acid sequence of SEQ ID NO:84; (b) HVR-H2 comprising the amino acid sequence of SEQ ID NO:119; (c) HVR-H3 comprising the amino acid sequence of SEQ ID NO:154; (d) HVR-L1 comprising the amino acid sequence of SEQ ID NO:187; (e) HVR-L2 comprising the amino acid sequence of SEQ ID NO:215; and (f) HVR-L3 comprising the amino acid sequence of SEQ ID NO:243; (a) HVR-H1 comprising the amino acid sequence of SEQ ID NO:84; (b) HVR-H2 comprising the amino acid sequence of SEQ ID NO:119; (c) HVR-H3 comprising the amino acid sequence of SEQ ID NO:154; (d) HVR-L1 comprising the amino acid sequence of SEQ ID NO:182; (e) HVR-L2 comprising the amino acid sequence of SEQ ID NO:216; and (f) HVR-L3 comprising the amino acid sequence of SEQ ID NO:238; (a) HVR-H1 comprising the amino acid sequence of SEQ ID NO:87; (b) HVR-H2 comprising the amino acid sequence of SEQ ID NO:120; (c) HVR-H3 comprising the amino acid sequence of SEQ ID NO:155; (d) HVR-L1 comprising the amino acid sequence of SEQ ID NO:182; (e) HVR-L2 comprising the amino acid sequence of SEQ ID NO:216; and (f) HVR-L3 comprising the amino acid sequence of SEQ ID NO:238; (a) HVR-H1 comprising the amino acid sequence of SEQ ID NO:88; (b) HVR-H2 comprising the amino acid sequence of SEQ ID NO:121; (c) HVR-H3 comprising the amino acid sequence of SEQ ID NO:156; (d) HVR-L1 comprising the amino acid sequence of SEQ ID NO:188; (e) HVR-L2 comprising the amino acid sequence of SEQ ID NO:217; and (f) HVR-L3 comprising the amino acid sequence of SEQ ID NO:244; (a) HVR-H1 comprising the amino acid sequence of SEQ ID NO:89; (b) HVR-H2 comprising the amino acid sequence of SEQ ID NO:122; (c) HVR-H3 comprising the amino acid sequence of SEQ ID NO:157; (d) HVR-L1 comprising the amino acid sequence of SEQ ID NO:189; (e) HVR-L2 comprising the amino acid sequence of SEQ ID NO:218; and (f) HVR-L3 comprising the amino acid sequence of SEQ ID NO:245; (a) HVR-H1 comprising the amino acid sequence of SEQ ID NO:90; (b) HVR-H2 comprising the amino acid sequence of SEQ ID NO:123; (c) HVR-H3 comprising the amino acid sequence of SEQ ID NO:158; (d) HVR-L1 comprising the amino acid sequence of SEQ ID NO:190; (e) HVR-L2 comprising the amino acid sequence of SEQ ID NO:219; and (f) HVR-L3 comprising the amino acid sequence of SEQ ID NO:246; (a) HVR-H1 comprising the amino acid sequence of SEQ ID NO:91; (b) HVR-H2 comprising the amino acid sequence of SEQ ID NO:124; (c) HVR-H3 comprising the amino acid sequence of SEQ ID NO:159; (d) HVR-L1 comprising the amino acid sequence of SEQ ID NO:191; (e) HVR-L2 comprising the amino acid sequence of SEQ ID NO:220; and (f) HVR-L3 comprising the amino acid sequence of SEQ ID NO:247; (a) HVR-H1 comprising the amino acid sequence of SEQ ID NO:92; (b) HVR-H2 comprising the amino acid sequence of SEQ ID NO:125; (c) HVR-H3 comprising the amino acid sequence of SEQ ID NO:160; (d) HVR-L1 comprising the amino acid sequence of SEQ ID NO:192; (e) HVR-L2 comprising the amino acid sequence of SEQ ID NO:221; and (f) HVR-L3 comprising the amino acid sequence of SEQ ID NO:248; (a) HVR-H1 comprising the amino acid sequence of SEQ ID NO:93; (b) HVR-H2 comprising the amino acid sequence of SEQ ID NO:126; (c) HVR-H3 comprising the amino acid sequence of SEQ ID NO:155; (d) HVR-L1 comprising the amino acid sequence of SEQ ID NO:193; (e) HVR-L2 comprising the amino acid sequence of SEQ ID NO:210; and (f) HVR-L3 comprising the amino acid sequence of SEQ ID NO:238; (a) HVR-H1 comprising the amino acid sequence of SEQ ID NO:94; (b) HVR-H2 comprising the amino acid sequence of SEQ ID NO:127; (c) HVR-H3 comprising the amino acid sequence of SEQ ID NO:161; (d) HVR-L1 comprising the amino acid sequence of SEQ ID NO:194; (e) HVR-L2 comprising the amino acid sequence of SEQ ID NO:220; and (f) HVR-L3 comprising the amino acid sequence of SEQ ID NO:249; (a) HVR-H1 comprising the amino acid sequence of SEQ ID NO:95; (b) HVR-H2 comprising the amino acid sequence of SEQ ID NO:128; (c) HVR-H3 comprising the amino acid sequence of SEQ ID NO:148; (d) HVR-L1 comprising the amino acid sequence of SEQ ID NO:195; (e) HVR-L2 comprising the amino acid sequence of SEQ ID NO:210; and (f) HVR-L3 comprising the amino acid sequence of SEQ ID NO:250; (a) HVR-H1 comprising the amino acid sequence of SEQ ID NO:78; (b) HVR-H2 comprising the amino acid sequence of SEQ ID NO:129; (c) HVR-H3 comprising the amino acid sequence of SEQ ID NO:162; (d) HVR-L1 comprising the amino acid sequence of SEQ ID NO:196; (e) HVR-L2 comprising the amino acid sequence of SEQ ID NO:223; and (f) HVR-L3 comprising the amino acid sequence of SEQ ID NO:251; (a) HVR-H1 comprising the amino acid sequence of SEQ ID NO:96; (b) HVR-H2 comprising the amino acid sequence of SEQ ID NO:130; (c) HVR-H3 comprising the amino acid sequence of SEQ ID NO:163; (d) HVR-L1 comprising the amino acid sequence of SEQ ID NO:197; (e) HVR-L2 comprising the amino acid sequence of SEQ ID NO:214; and (f) HVR-L3 comprising the amino acid sequence of SEQ ID NO:252; (a) HVR-H1 comprising the amino acid sequence of SEQ ID NO:97; (b) HVR-H2 comprising the amino acid sequence of SEQ ID NO:131; (c) HVR-H3 comprising the amino acid sequence of SEQ ID NO:164; (d) HVR-L1 comprising the amino acid sequence of SEQ ID NO:198; (e) HVR-L2 comprising the amino acid sequence of SEQ ID NO:209; and (f) HVR-L3 comprising the amino acid sequence of SEQ ID NO:253; (a) HVR-H1 comprising the amino acid sequence of SEQ ID NO:98; (b) HVR-H2 comprising the amino acid sequence of SEQ ID NO:132; (c) HVR-H3 comprising the amino acid sequence of SEQ ID NO:165; (d) HVR-L1 comprising the amino acid sequence of SEQ ID NO:199; (e) HVR-L2 comprising the amino acid sequence of SEQ ID NO:225; and (f) HVR-L3 comprising the amino acid sequence of SEQ ID NO:254; (a) HVR-H1 comprising the amino acid sequence of SEQ ID NO:99; (b) HVR-H2 comprising the amino acid sequence of SEQ ID NO:133; (c) HVR-H3 comprising the amino acid sequence of SEQ ID NO:166; (d) HVR-L1 comprising the amino acid sequence of SEQ ID NO:200; (e) HVR-L2 comprising the amino acid sequence of SEQ ID NO:226; and (f) HVR-L3 comprising the amino acid sequence of SEQ ID NO:255; (a) HVR-H1 comprising the amino acid sequence of SEQ ID NO:100; (b) HVR-H2 comprising the amino acid sequence of SEQ ID NO:134; (c) HVR-H3 comprising the amino acid sequence of SEQ ID NO:167; (d) HVR-L1 comprising the amino acid sequence of SEQ ID NO:179; (e) HVR-L2 comprising the amino acid sequence of SEQ ID NO:211; and (f) HVR-L3 comprising the amino acid sequence of SEQ ID NO:256; (a) HVR-H1 comprising the amino acid sequence of SEQ ID NO:101; (b) HVR-H2 comprising the amino acid sequence of SEQ ID NO:135; (c) HVR-H3 comprising the amino acid sequence of SEQ ID NO:168; (d) HVR-L1 comprising the amino acid sequence of SEQ ID NO:201; (e) HVR-L2 comprising the amino acid sequence of SEQ ID NO:227; and (f) HVR-L3 comprising the amino acid sequence of SEQ ID NO:257; (a) HVR-H1 comprising the amino acid sequence of SEQ ID NO:102; (b) HVR-H2 comprising the amino acid sequence of SEQ ID NO:136; (c) HVR-H3 comprising the amino acid sequence of SEQ ID NO:169; (d) HVR-L1 comprising the amino acid sequence of SEQ ID NO:202; (e) HVR-L2 comprising the amino acid sequence of SEQ ID NO:228; and (f) HVR-L3 comprising the amino acid sequence of SEQ ID NO:258; (a) HVR-H1 comprising the amino acid sequence of SEQ ID NO:96; (b) HVR-H2 comprising the amino acid sequence of SEQ ID NO:137; (c) HVR-H3 comprising the amino acid sequence of SEQ ID NO:170; (d) HVR-L1 comprising the amino acid sequence of SEQ ID NO:203; (e) HVR-L2 comprising the amino acid sequence of SEQ ID NO:229; and (f) HVR-L3 comprising the amino acid sequence of SEQ ID NO:259; (a) HVR-H1 comprising the amino acid sequence of SEQ ID NO:79; (b) HVR-H2 comprising the amino acid sequence of SEQ ID NO:138; (c) HVR-H3 comprising the amino acid sequence of SEQ ID NO:171; (d) HVR-L1 comprising the amino acid sequence of SEQ ID NO:204; (e) HVR-L2 comprising the amino acid sequence of SEQ ID NO:230; and (f) HVR-L3 comprising the amino acid sequence of SEQ ID NO:260; (a) HVR-H1 comprising the amino acid sequence of SEQ ID NO:103; (b) HVR-H2 comprising the amino acid sequence of SEQ ID NO:139; (c) HVR-H3 comprising the amino acid sequence of SEQ ID NO:172; (d) HVR-L1 comprising the amino acid sequence of SEQ ID NO:205; (e) HVR-L2 comprising the amino acid sequence of SEQ ID NO:226; and (f) HVR-L3 comprising the amino acid sequence of SEQ ID NO:255; (a) HVR-H1 comprising the amino acid sequence of SEQ ID NO:104; (b) HVR-H2 comprising the amino acid sequence of SEQ ID NO:140; (c) HVR-H3 comprising the amino acid sequence of SEQ ID NO:173; (d) HVR-L1 comprising the amino acid sequence of SEQ ID NO:206; (e) HVR-L2 comprising the amino acid sequence of SEQ ID NO:227; and (f) HVR-L3 comprising the amino acid sequence of SEQ ID NO:261; (a) HVR-H1 comprising the amino acid sequence of SEQ ID NO:105; (b) HVR-H2 comprising the amino acid sequence of SEQ ID NO:141; (c) HVR-H3 comprising the amino acid sequence of SEQ ID NO:174; (d) HVR-L1 comprising the amino acid sequence of SEQ ID NO:207; (e) HVR-L2 comprising the amino acid sequence of SEQ ID NO:231; and (f) HVR-L3 comprising the amino acid sequence of SEQ ID NO:262; and (a) HVR-H1 comprising the amino acid sequence of SEQ ID NO:106; (b) HVR-H2 comprising the amino acid sequence of SEQ ID NO:142; (c) HVR-H3 comprising the amino acid sequence of SEQ ID NO:175; (d) HVR-L1 comprising the amino acid sequence of SEQ ID NO:208; (e) HVR-L2 comprising the amino acid sequence of SEQ ID NO:209; and (f) HVR-L3 comprising the amino acid sequence of SEQ ID NO:263.

In another aspect, an anti-TMEM106B antibody of the present disclosure comprises a heavy chain variable domain (V_{H}) sequence having at least 90%, 91%, 92%, 93%, 94%, 95%, 96%, 97%, 98%, 99%, or 100% sequence identity to an amino acid sequence selected from the group consisting of SEQ ID NOs:4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40. In certain embodiments, a V_{H} sequence having at least 90%, 91%, 92%, 93%, 94%, 95%, 96%, 97%, 98%, or 99% identity to an amino acid sequence selected from the group consisting of SEQ ID NOs: 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40 contains substitutions (e.g., conservative substitutions), insertions, or deletions relative to the reference sequence, but an anti-TMEM106B antibody comprising that sequence retains the ability to bind to TMEM106B. In certain embodiments, a total of 1 to 10 amino acids have been substituted, inserted, and/or deleted in SEQ ID NO: 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40. In certain embodiments, a total of 1 to 5 amino acids have been substituted, inserted and/or deleted in SEQ ID NO: 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40. In certain embodiments, substitutions, insertions, or deletions occur in regions outside the HVRs (i.e., in the FRs). Optionally, the anti-TMEM106B antibody comprises the V_{H} sequence of SEQ ID NO: 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, including post-translational modifications of that sequence. In a particular embodiment, the V_{H} comprises one, two or three HVRs selected from: (a) HVR-H1 comprising an amino acid sequence selected from the group consisting of SEQ ID NOs:77, 78, 79, 80, 81, 82, 83, 84, 85, 86, 87, 88, 89, 90, 91, 92, 93, 94, 95, 96, 97, 98, 99, 100, 101, 102, 103, 104, 105, and 106; (b) HVR-H2 comprising an amino acid sequence selected from the group consisting of SEQ ID NOs:107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, and 142; (c) HVR-H3 comprising an amino acid sequence selected from the group consisting of SEQ ID NOs:143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, and 175.

In another aspect, an anti-TMEM106B antibody of the present disclosure comprises a light chain variable domain (V_{L}) sequence having at least 90%, 91%, 92%, 93%, 94%, 95%, 96%, 97%, 98%, 99%, or 100% sequence identity to an amino acid sequence selected from the group consisting of SEQ ID NOs: 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, and 76. In certain embodiments, a V_{L} sequence having at least 90%, 91%, 92%, 93%, 94%, 95%, %%, 97%, 98%, or 99% identity to an amino acid sequence selected from the group consisting of SEQ ID NOs: 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, and 76 contains substitutions (e.g., conservative substitutions), insertions, or deletions relative to the reference sequence, but an anti-TMEM106B antibody comprising that sequence retains the ability to bind to TMEM106B. In some embodiments, a total of 1 to 10 amino acids have been substituted, inserted and/or deleted in SEQ ID NO: 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or 76. In certain embodiments, a total of 1 to 5 amino acids have been substituted, inserted and/or deleted in SEQ ID NO: 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or 76. In certain embodiments, the substitutions, insertions, or deletions occur in regions outside the HVRs (i.e., in the FRs). Optionally, the anti-TMEM106B antibody comprises the V_{L} sequence of SEQ ID NO: 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or 76, including post-translational modifications of that sequence. In a particular embodiment, the V_{L} comprises one, two or three HVRs selected from: (a) HVR-L1 comprising an amino acid sequence selected from the group consisting of SEQ ID NOs:176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, and 208; (b) HVR-L2 comprising an amino acid sequence selected from the group consisting of SEQ ID NOs:209, 210, 211, 212, 213, 214, 215, 216, 217, 218, 219, 220, 221, 222, 223, 224, 225, 226, 227, 228, 229, 230, and 231; and (c) HVR-L3 comprising an amino acid sequence selected from the group consisting of SEQ ID NOs:232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, and 263.

In some embodiments, an anti-TMEM106B antibody is provided, wherein the antibody comprises a V_{H} as in any of the embodiments provided above, and a V_{L} as in any of the embodiments provided above. In some embodiments, provided herein are anti-TMEM106B antibodies, wherein the antibody comprises a V_{H} as in any of the embodiments provided above, and a V_{L} as in any of the embodiments provided above. In one embodiment, the antibody comprises the V_{H} and V_{L} sequences in SEQ ID NOs: 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40 and SEQ ID NOs: 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, and 76, respectively, including post-translational modifications of those sequences.

In some embodiments, provided herein are anti-TMEM106B antibodies comprising a heavy chain variable domain (V_{H}) and a light chain variable domain (V_{L}), wherein the V_{H} and V_{L} are selected from the group consisting of: V_{H} comprising the amino acid sequence of SEQ ID NO:4 and V_{L} comprising the amino acid sequence of SEQ ID NO:41; V_{H} comprising the amino acid sequence of SEQ ID NO:5 and V_{L} comprising the amino acid sequence of SEQ ID NO:42; V_{H} comprising the amino acid sequence of SEQ ID NO:6 and V_{L} comprising the amino acid sequence of SEQ ID NO:43; V_{H} comprising the amino acid sequence of SEQ ID NO:7 and V_{L} comprising the amino acid sequence of SEQ ID NO:44; V_{H} comprising the amino acid sequence of SEQ ID NO:8 and V_{L} comprising the amino acid sequence of SEQ ID NO:45; V_{H} comprising the amino acid sequence of SEQ ID NO:9 and V_{L} Comprising the amino acid sequence of SEQ ID NO:46; V_{H} comprising the amino acid sequence of SEQ ID NO:10 and V_{L} comprising the amino acid sequence of SEQ ID NO:47; V_{H} comprising the amino acid sequence of SEQ ID NO:11 and V_{L} comprising the amino acid sequence of SEQ ID NO:48; V_{H} Comprising the amino acid sequence of SEQ ID NO:12 and V_{L} comprising the amino acid sequence of SEQ ID NO:49; V_{H} comprising the amino acid sequence of SEQ ID NO:13 and V_{L} comprising the amino acid sequence of SEQ ID NO:50; V_{H} comprising the amino acid sequence of SEQ ID NO:14 and V_{L} comprising the amino acid sequence of SEQ ID NO:51; V_{H} comprising the amino acid sequence of SEQ ID NO:15 and V_{L} comprising the amino acid sequence of SEQ ID NO:52; V_{H} comprising the amino acid sequence of SEQ ID NO:16 and V_{L} comprising the amino acid sequence of SEQ ID NO:53; V_{H} Comprising the amino acid sequence of SEQ ID NO:17 and V_{L} comprising the amino acid sequence of SEQ ID NO:54; V_{H} comprising the amino acid sequence of SEQ ID NO:18 and V_{L} comprising the amino acid sequence of SEQ ID NO:54; V_{H} comprising the amino acid sequence of SEQ ID NO:19 and V_{L} comprising the amino acid sequence of SEQ ID NO:55; V_{H} comprising the amino acid sequence of SEQ ID NO:20 and V_{L} comprising the amino acid sequence of SEQ ID NO:56; V_{H} comprising the amino acid sequence of SEQ ID NO:21 and V_{L} comprising the amino acid sequence of SEQ ID NO:57; V_{H} Comprising the amino acid sequence of SEQ ID NO:22 and V_{L} comprising the amino acid sequence of SEQ ID NO:58; V_{H} comprising the amino acid sequence of SEQ ID NO:23 and V_{L} comprising the amino acid sequence of SEQ ID NO:59; V_{H} comprising the amino acid sequence of SEQ ID NO:24 and V_{L} comprising the amino acid sequence of SEQ ID NO:60; V_{H} comprising the amino acid sequence of SEQ ID NO:25 and V_{L} comprising the amino acid sequence of SEQ ID NO:61; V_{H} comprising the amino acid sequence of SEQ ID NO:26 and V_{L} comprising the amino acid sequence of SEQ ID NO:62; V_{H} Comprising the amino acid sequence of SEQ ID NO:27 and V_{L} comprising the amino acid sequence of SEQ ID NO:63; V_{H} comprising the amino acid sequence of SEQ ID NO:28 and V_{L} comprising the amino acid sequence of SEQ ID NO:64; V_{H} comprising the amino acid sequence of SEQ ID NO:29 and V_{L} comprising the amino acid sequence of SEQ ID NO:65; V_{H} comprising the amino acid sequence of SEQ ID NO:30 and V_{L} comprising the amino acid sequence of SEQ ID NO:66; V_{H} comprising the amino acid sequence of SEQ ID NO:31 and V_{L} comprising the amino acid sequence of SEQ ID NO:67; V_{H} Comprising the amino acid sequence of SEQ ID NO:32 and V_{L} comprising the amino acid sequence of SEQ ID NO:68; V_{H} comprising the amino acid sequence of SEQ ID NO:33 and V_{L} comprising the amino acid sequence of SEQ ID NO:69; V_{H} comprising the amino acid sequence of SEQ ID NO:34 and V_{L} comprising the amino acid sequence of SEQ ID NO:70; V_{H} comprising the amino acid sequence of SEQ ID NO:35 and V_{L} comprising the amino acid sequence of SEQ ID NO:71; V_{H} comprising the amino acid sequence of SEQ ID NO:36 and V_{L} comprising the amino acid sequence of SEQ ID NO:72; V_{H} comprising the amino acid sequence of SEQ ID NO:37 and V_{L} comprising the amino acid sequence of SEQ ID NO:73; V_{H} comprising the amino acid sequence of SEQ ID NO:38 and V_{L} comprising the amino acid sequence of SEQ ID NO:74; V_{H} comprising the amino acid sequence of SEQ ID NO:39 and V_{L} comprising the amino acid sequence of SEQ ID NO:75; and V_{H} comprising the amino acid sequence of SEQ ID NO:40 and V_{L} comprising the amino acid sequence of SEQ ID NO:76.

In some embodiments, an anti-TMEM106B antibody of the present disclosure competitively inhibits binding of at least one reference antibody comprising the V_{H} and V_{L} (e.g., as shown in Table 4 below) of TM-54, TM-56, TM-59, TM-60, TM-61, TM-62, TM-63, TM-64, TM-65, TM-66, TM-67, TM-68, TM-69, TM-70, TM-71, TM-72, TM-73, TM-74, TM-75, TM-76, TM-77, TM-78, TM-79, TM-80. TM-81, TM-82, TM-83, TM-84, TM-85, TM-86, TM-87, TM-88, TM-89, TM90, TM-91, TM-92, TM-93, TM-94, or any combination thereof, for binding to TMEM106B.

In some embodiments, an anti-TMEM106B antibody of the present disclosure binds to an epitope of human TMEM106B that is the same or overlaps with the TMEM106B epitope bound by at least one antibody comprising the V_{H} and V_{L} (e.g., as shown in Table 4 below) of TM-54, TM-56, TM-59, TM-60, TM-61, TM-62, TM-63, TM-64, TM-65, TM-66, TM-67, TM-68, TM-69, TM-70, TM-71, TM-72, TM-73, TM-74, TM-75, TM-76, TM-77, TM-78, TM-79, TM-80. TM-81, TM-82, TM-83, TM-84, TM-85, TM-86, TM-87, TM-88, TM-89, TM90, TM-91, TM-92, TM-93, TM-94, or any combination thereof, for binding to TMEM106B. An antibody that binds to the “same epitope” of TMEM106B as a reference antibody contacts all of the same residues of TMEM106B as the reference antibody. An antibody that binds to an “overlapping” epitope of TMEM106B as a reference antibody contacts at least some of the same residues of TMEM106B as the reference antibody. Detailed exemplary methods for mapping an epitope to which an antibody binds are provided in Morris (1996) “Epitope Mapping Protocols,” in Methods in Molecular Biology vol. 66 (Humana Press, Totowa, NJ).

Any suitable competition assay or TMEM106B binding assay known in the art, such as BIAcore analysis, ELISA assays, or flow cytometry, may be utilized to determine whether an anti-TMEM106B antibody competes with (or competitively inhibits the binding of) one or more antibodies comprising the V_{H} and V_{L} (e.g., as shown in Table 4 below) of TM-54, TM-56, TM-59, TM-60, TM-61, TM-62, TM-63, TM-64, TM-65, TM-66, TM-67, TM-68, TM-69, TM-70, TM-71, TM-72, TM-73, TM-74, TM-75, TM-76, TM-77, TM-78, TM-79, TM-80. TM-81, TM-82, TM-83, TM-84, TM-85, TM-86, TM-87, TM-88, TM-89, TM90, TM-91, TM-92, TM-93, TM-94, or any combination thereof for binding to TMEM106B. In an exemplary competition assay, immobilized TMEM106B or cells expressing TMEM106B on the cell surface are incubated in a solution comprising a first labeled antibody that binds to TMEM106B (e.g., human or non-human primate) and a second unlabeled antibody that is being tested for its ability to compete with the first antibody for binding to TMEM106B. The second antibody may be present in a hybridoma supernatant. As a control, immobilized TMEM106B or cells expressing TMEM106B is incubated in a solution comprising the first labeled antibody but not the second unlabeled antibody. After incubation under conditions permissive for binding of the first antibody to TMEM106B, excess unbound antibody is removed, and the amount of label associated with immobilized TMEM106B or cells expressing TMEM106B is measured. If the amount of label associated with immobilized TMEM106B or cells expressing TMEM106B is substantially reduced in the test sample relative to the control sample, then that indicates that the second antibody is competing with the first antibody for binding to TMEM106B. See, Harlow and Lane (1988) Antibodies: A Laboratory Manual ch. 14 (Cold Spring Harbor Laboratory, Cold Spring Harbor, NY).

Anti-TMEM106B antibodies of the present disclosure may bind to various regions of TMEM106B, including various regions of human TMEM106B. Such regions of TMEM106B include the cytoplasmic domain of TMEM106B or the lumenal domain TMEM106B.

In some embodiments, an anti-TMEM106B antibody of the present disclosure binds to one or more regions or domains of TMEM106B. In some embodiments, an anti-TMEM106B antibody of the present disclosure binds to one or more regions or domains of human TMEM106B.

In some embodiments, the anti-TMEM106B antibody according to any of the above embodiments is a monoclonal antibody, including a humanized and/or human antibody. In some embodiments, the anti-TMEM106B antibody is an antibody fragment, e.g., a Fv, Fab, Fab′, scFv, diabody, or F(ab′)2 fragment. In some embodiments, the anti-TMEM106B antibody is a substantially full-length antibody, e.g., an IgG1 antibody, IgG2a antibody or other antibody class or isotype as defined herein.

In some embodiments, an anti-TMEM106B antibody according to any of the above embodiments may incorporate any of the features, singly or in combination, as described in Sections 1-7 below:

### (1) Anti-TMFM06B Antibody Binding Affinity

In some embodiments of any of the antibodies provided herein, the antibody has a dissociation constant (Kd) of <1 μM, <100 nM, <10 nM, <1 nM, <0.1 nM, <0.01 nM, or <0.001 nM (e.g., 10⁻⁸ M or less, e.g., from 10⁻⁸ M to 10⁻¹³ M, e.g., from 10⁻⁹ M to 10⁻¹³ M). Dissociation constants may be determined through any analytical technique, including any biochemical or biophysical technique such as ELISA, surface plasmon resonance (SPR), bio-layer interferometry (see, e.g., Octet System by ForteBio), isothermal titration calorimetry (ITC), differential scanning calorimetry (DSC), circular dichroism (CD), stopped-flow analysis, and colorimetric or fluorescent protein melting analyses. In one embodiment, Kd is measured by a radiolabeled antigen binding assay (RIA). In some embodiment, an RIA is performed with the Fab version of an antibody of interest and its antigen, for example as described in Chen et al. J. Mol. Biol. 293:865-881(1999)). In some embodiments, Kd is measured using a BIACORE surface plasmon resonance assay, for example, an assay using a BIACORE-2000 or a BIACORE-3000 (BIAcore, Inc., Piscataway, NJ) is performed at 25° C. with immobilized antigen CM5 chips at ˜10 response units (RU). In some embodiments, the K_{D} is determined using a monovalent antibody (e.g., a Fab) or a full-length antibody. In some embodiments, the K_{D} is determined using a full-length antibody in a monovalent form.

In some embodiments, an anti-TMEM106B antibody of the present disclosure may have nanomolar or even picomolar affinities for TMEM106B. In some embodiments, the dissociation constant (Kd) of the antibody is about 0.1 nM to about 500 nM. For example, the Kd of the antibody is any of about 500 nM, about 400 nM, about 300 nM, about 200 nM, about 100 nM, about 75 nM, about 50 nM, about 25 nM, about 10 nM, about 9 nM, about 8 nM, about 7 nM, about 6 nM, about 5 nM, about 4 nM, about 3 nM, about 2 nM, about nM, or about 1 nM to about 0.1 nM for binding to human TMEM106B.

### (2) Antibody Fragments

In some embodiments of any of the antibodies provided herein, the antibody is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see. e.g., WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP404097; WO 1993/01161; Hudson et al. Nat. Med. 9:129-134 (2003). Triabodies and tetrabodies are also described in Hudson et al. Nat. Med. 9:129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (see, e.g., U.S. Pat. No. 6,248,516).

Antibody fragments can be made by various techniques, including but not limited to proteolytic digestion of an intact antibody as well as production by recombinant host cells (e.g., E. coli or phage), as described herein.

### (3) Chimeric and Humanized Antibodies

In some embodiments of any of the antibodies provided herein, the antibody is a chimeric antibody. Certain chimeric antibodies are described, e.g., in U.S. Pat. No. 4,816,567. In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In some embodiments of any of the antibodies provided herein, the antibody is a humanized antibody. Typically, a non-human antibody is humanized to reduce immunogenicity to humans, while retaining the specificity and affinity of the parental non-human antibody. In certain embodiments, a humanized antibody is substantially non-immunogenic in humans. In certain embodiments, a humanized antibody has substantially the same affinity for a target as an antibody from another species from which the humanized antibody is derived. See, e.g., U.S. Pat. Nos. 5,530,101, 5,693,761; 5,693,762; and 5,585,089. In certain embodiments, amino acids of an antibody variable domain that can be modified without diminishing the native affinity of the antigen binding domain while reducing its immunogenicity are identified. See, e.g., U.S. Pat. Nos. 5,766,886 and 5,869,619. Generally, a humanized antibody comprises one or more variable domains in which HVRs (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), for example, to restore or improve antibody specificity or affinity.

Humanized antibodies and methods of making them are reviewed, for example, in Almagro et al. Front. Biosci. 13:161 9-1633 (2008), and are further described, e.g., in U.S. Pat. Nos. 5,821,337, 7,527,791, 6,982,321, and 7,087,409. Human framework regions that may be used for humanization include but are not limited to: framework regions selected using the “best-fit” method (see. e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA 89:4285 (1992); and Presta et al., J. Immunol. 151:2623 (1993)); human mature (somatically mutated) framework regions or human germline framework regions (see, e.g., Almagro and Fransson Front. Biosci. 13:1619-1633 (2008)); and framework regions derived from screening FR libraries (see, e.g., Baca et al. J. Biol. Chem. 272:10678-10684 (1997) and Rosok et al. J. Biol. Chem. 271:22611-22618 (1996)).

### (4) Human Antibodies

In some embodiments of any of the antibodies provided herein, the antibody is a human antibody. Human antibodies can be produced using various techniques known in the art. Human antibodies are described generally in van Dijk et al. Curr. Opin. Pharmacol. 5:368-74 (2001) and Lonberg Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. One can engineer mouse strains deficient in mouse antibody production with large fragments of the human Ig loci in anticipation that such mice would produce human antibodies in the absence of mouse antibodies. Large human Ig fragments can preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains can yield high affinity fully human antibodies against any antigen of interest, including human antigens. Using the hybridoma technology, antigen-specific human monoclonal antibodies with the desired specificity can be produced and selected. Certain exemplary methods are described in U.S. Pat. No. 5,545,807, EP 546073, and EP 546073. See also, for example, U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology. Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See. e.g., Kozbor J. Immunol. 133:3001 (1984) and Boemer et al. J. Immunol. 147:86 (1991)). Human antibodies generated via human B-cell hybridoma technology are also described in Li et al. Proc. Natl. Acad. Sci. USA, 1 03:3557-3562 (2006). Additional methods include those described, for example, in U.S. Pat. No. 7,189,826 (describing production of monoclonal human IgM antibodies from hybridoma cell lines). Human hybridoma technology (Trioma technology) is also described in Vollmers et al. Histology and Histopathology 20(3):927-937 (2005) and Vollmers et al. Methods and Findings in Experimental and Clinical Pharmacology 27(3):185-91 (2005). Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

In some embodiments of any of the antibodies provided herein, the antibody is a human antibody isolated by in vitro methods and/or screening combinatorial libraries for antibodies with the desired activity or activities. Suitable examples include but are not limited to phage display (CAT, Morphosys, Dyax, Biosite/Medarex, Xoma, Symphogen, Alexion (formerly Proliferon), Affimed) ribosome display (CAT), yeast display (Adimab), and the like. In certain phage display methods, repertoires of VH and VL genes are separately cloned by polymerase chain reaction (PCR) and recombined randomly in phage libraries, which can then be screened for antigen-binding phage as described in Winter et al. Ann. Rev. Immunol. 12: 433-455 (1994). For example, a variety of methods are known in the art for generating phage display libraries and screening such libraries for antibodies possessing the desired binding characteristics. See also Sidhu et al. J. Mol. Biol. 338(2): 299-310, 2004; Lee et al. J. Mol. Biol. 340(5): 1073-1093, 2004; Fellouse Proc. Natl. Acad. Sci. USA 101(34):12467-12472 (2004); and Lee et al. J. Immunol. Methods 284(-2):1 19-132 (2004). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al. EMBO J. 12: 725-734 (1993). Finally, naive libraries can also be made synthetically by cloning unrearranged V-gene segments from stem cells, and using PCR primers comprising random sequence to encode the highly variable HVR3 regions and to accomplish rearrangement in vitro, as described by Hoogenboom et al. J. Mol. Biol., 227: 381-388, 1992. Patent publications describing human antibody phage libraries include, for example: U.S. Pat. No. 5,750,373, and US Patent Publication Nos. 2007/0292936 and 2009/0002360. Antibodies isolated from human antibody libraries are considered human antibodies or human antibody fragments herein.

### (5) Constant Regions Including Fc Regions

In some embodiments of any of the antibodies provided herein, the antibody comprises an Fc. In some embodiments, the Fc is a human IgG1, IgG2, IgG3, and/or IgG4 isotype. In some embodiments, the antibody is of the IgG class, the IgM class, or the IgA class.

In certain embodiments of any of the antibodies provided herein, the antibody has an IgG2 isotype. In some embodiments, the antibody contains a human IgG2 constant region. In some embodiments, the human IgG2 constant region includes an Fc region. In some embodiments, the antibody induces the one or more TMEM106B activities or independently of binding to an Fc receptor. In some embodiments, the antibody binds an inhibitory Fc receptor. In certain embodiments, the inhibitory Fc receptor is inhibitory Fc-gamma receptor IIB (FcγIIB).

In certain embodiments of any of the antibodies provided herein, the antibody has an IgG1 isotype. In some embodiments, the antibody contains a mouse IgG1 constant region. In some embodiments, the antibody contains a human IgG1 constant region. In some embodiments, the human IgG1 constant region includes an Fc region. In some embodiments, the antibody binds an inhibitory Fc receptor. In certain embodiments, the inhibitory Fc receptor is inhibitory Fc-gamma receptor IIB (FcγIIB).

In certain embodiments of any of the antibodies provided herein, the antibody has an IgG4 isotype. In some embodiments, the antibody contains a human IgG4 constant region. In some embodiments, the human IgG4 constant region includes an Fc region. In some embodiments, the antibody binds an inhibitory Fc receptor. In certain embodiments, the inhibitory Fc receptor is inhibitory Fc-gamma receptor IIB (FcγIIB).

In certain embodiments of any of the antibodies provided herein, the antibody has a hybrid IgG2/4 isotype. In some embodiments, the antibody includes an amino acid sequence comprising amino acids 118 to 260 according to EU numbering of human IgG2 and amino acids 261-447 according to EU numbering of human IgG4 (WO 1997/11971; WO 2007/106585).

In some embodiments, the Fc region increases clustering without activating complement as compared to a corresponding antibody comprising an Fc region that does not comprise the amino acid substitutions. In some embodiments, the antibody induces one or more activities of a target specifically bound by the antibody. In some embodiments, the antibody binds to TMEM106B.

It may also be desirable to modify an anti-TMEM106B antibody of the present disclosure to modify effector function and/or to increase serum half-life of the antibody. For example, the Fc receptor binding site on the constant region may be modified or mutated to remove or reduce binding affinity to certain Fc receptors, such as FcγRI, FcγRII, and/or FcγRIII to reduce Antibody-dependent cell-mediated cytotoxicity. In some embodiments, the effector function is impaired by removing N-glycosylation of the Fc region (e.g., in the CH2 domain of IgG) of the antibody. In some embodiments, the effector function is impaired by modifying regions such as 233-236, 297, and/or 327-331 of human IgG as described in WO 99/58572 and Armour et al. Molecular Immunology 40: 585-593 (2003); Reddy et al. J. Immunology 164:1925-1933 (2000). In other embodiments, it may also be desirable to modify an anti-TMEM106B antibody of the present disclosure to modify effector function to increase finding selectivity toward the ITIM-containing FcgRIIb (CD32b) to increase clustering of TMEM106B antibodies on adjacent cells without activating humoral responses including Antibody-dependent cell-mediated cytotoxicity and antibody-dependent cellular phagocytosis.

To increase the serum half-life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule. Other amino acid sequence modifications.

### (6) Multispecific Antibodies

Multispecific antibodies are antibodies that have binding specificities for at least two different epitopes, including those on the same or another polypeptide (e.g., one or more TMEM106B polypeptides of the present disclosure). In some embodiments, the multispecific antibody can be a bispecific antibody. In some embodiments, the multispecific antibody can be a trispecific antibody. In some embodiments, the multispecific antibody can be a tetraspecific antibody. Such antibodies can be derived from full-length antibodies or antibody fragments (e.g., F(ab′)₂ bispecific antibodies). In some embodiments, the multispecific antibody comprises a first antigen binding region which binds to first site on TMEM106B and comprises a second antigen binding region which binds to a second site on TMEM106B. In some embodiment, the multispecific antibodies comprises a first antigen binding region which binds to TMEM106B and a second antigen binding region that binds to a second polypeptide.

Provided herein are multispecific antibodies comprises a first antigen binding region, wherein the first antigen binding region comprises the six HVRs of an antibody described herein, which binds to TMEM106B and a second antigen binding region that binds to a second polypeptide. In some embodiments, the first antigen binding region comprises the V_{H} or V_{L} of an antibody described herein.

In some embodiments of any of the multispecific antibodies, the second polypeptide is a) an antigen facilitating transport across the blood-brain-barrier; (b) an antigen facilitating transport across the blood-brain-barrier selected from transferrin receptor (TR), insulin receptor (HIR), insulin-like growth factor receptor (IGFR), low-density lipoprotein receptor related proteins 1 and 2 (LPR-1 and 2), diphtheria toxin receptor, CRM197, a llama single domain antibody, TMEM 30(A), a protein transduction domain, TAT, Syn-B, penetratin, a poly-arginine peptide, an angiopep peptide, and ANG1005; (c) a disease-causing protein selected from amyloid beta, oligomeric amyloid beta, amyloid beta plaques, amyloid precursor protein or fragments thereof, Tau, IAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin 1, ataxin 2, ataxin 3, ataxin 7, ataxin 8, ataxin 10, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides; (d) ligands and/or proteins expressed on immune cells, wherein the ligands and/or proteins selected from CD40, OX40, ICOS, CD28, CD137/4-1BB, CD27, GITR, PD-L1, CTLA-4, PD-L2, PD-1, B7-H3, B7-H4, HVEM, BTLA, KIR, GAL9, TIM3, A2AR, LAG-3, and phosphatidylserine; and/or (e) a protein, lipid, polysaccharide, or glycolipid expressed on one or more tumor cells and any combination thereof.

Numerous antigens are known in the art that facilitate transport across the blood-brain barrier (see, e.g., Gabathuler R. Neurobiol. Dis. 37:48-57 (2010)). Such second antigens include, without limitation, transferrin receptor (TR), insulin receptor (HIR), Insulin-like growth factor receptor (IGFR), low-density lipoprotein receptor related proteins 1 and 2 (LPR-1 and 2), diphtheria toxin receptor, including CRM197 (a non-toxic mutant of diphtheria toxin), llama single domain antibodies such as TMEM 30(A) (Flippase), protein transduction domains such as TAT, Syn-B, or penetratin, poly-arginine or generally positively charged peptides, Angiopep peptides such as ANG1005 (see, e.g., Gabathuler, 2010), and other cell surface proteins that are enriched on blood-brain barrier endothelial cells (see, e.g., Daneman et al. PLoS One 5(10):e13741 (2010)).

The multivalent antibodies may recognize the TMEM106B antigen as well as without limitation additional antigens Aβ peptide, antigen or an α-synuclein protein antigen or, Tau protein antigen or, TDP-43 protein antigen or, prion protein antigen or, huntingtin protein antigen, or RAN, translation Products antigen, including the DiPeptide Repeats, (DPRs peptides) composed of glycine-alanine (GA), glycine-proline (GP), glycine-arginine (GR), proline-alanine (PA), or proline-arginine (PR), Insulin receptor, insulin like growth factor receptor. Transferrin receptor or any other antigen that facilitate antibody transfer across the blood brain barrier. In some embodiments, the second polypeptide is transferrin. In some embodiments, the second polypeptide is Tau. In some embodiments, the second polypeptide is Aβ. In some embodiments, the second polypeptide is TREM2. In some embodiments, the second polypeptide is α-synuclein.

The multivalent antibody contains at least one polypeptide chain (and preferably two polypeptide chains), wherein the polypeptide chain or chains comprise two or more variable domains. For instance, the polypeptide chain or chains may comprise VD1-(X1)ₙ-VD2-(X2)ₙ-Fc, wherein VD1 is a first variable domain, VD2 is a second variable domain, Fc is one polypeptide chain of an Fc region, X1 and X2 represent an amino acid or polypeptide, and n is 0 or 1. Similarly, the polypeptide chain or chains may comprise V_{H}-C_{H}1-flexible linker-V_{H}-C_{H}1-Fc region chain; or V_{H}—C_{H}1-V_{H}-C_{H}1-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

Techniques for making multispecific antibodies include, but are not limited to, recombinant co-expression of two immunoglobulin heavy chain-light chain pairs having different specificities (see Milstein and Cuello Nature 305: 537 (1983), WO 93/08829, and Traunecker et al. EMBO J. 10:3655 (1991)), and “knob-in-hole” engineering (see. e.g., U.S. Pat. No. 5,731,168). See also WO 2013/026833 (CrossMab). Multi-specific antibodies may also be made by engineering electrostatic steering effects for making antibody Fc-heterodimeric molecules (WO 2009/089004A1); cross-linking two or more antibodies (see, e.g., U.S. Pat. No. 4,676,980); using leucine; using “diabody” technology for making bispecific antibody fragments (see, e.g., Hollinger et al. Proc. Natl. Acad. Sci. USA 90:6444-6448 (1993)); and using single-chain Fv (scFv) dimers (see, e.g., Gruber et al. J. Immunol. 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g., US 2006/0025576). The antibody herein also includes a “Dual Acting FAb” or “DAF” comprising an antigen binding site that binds to multiple TMEM106B (see, US 2008/0069820, for example).

### (7) Antibody Variants

In some embodiments of any of the antibodies provided herein, amino acid sequence variants of the antibodies are contemplated. For example, it may be desirable to improve the binding affinity and/or other biological properties of the antibody.

### (i) Substitution, Insertion, and Deletion Variants

In some embodiments of any of the antibodies provided herein, antibody variants having one or more amino acid substitutions are provided. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody.

TABLE A
Amino Acid Substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; | Leu |
| | Phe; Norleucine | |
| Leu (L) | Norleucine; Ile; Val; | Ile |
| | Met; Ala; Phe | |
| Lys (K) | Arg; Gln; Asn | Arg |
| Met (M) | Leu; Phe; Ile | Leu |
| Phe (F) | Leu; Val; Ile; Ala; Tyr | Tyr |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; | Leu |
| | Ala; Norleucine | |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro; and
(6) aromatic: Trp, Tyr, Phe.

For example, non-conservative substitutions can involve the exchange of a member of one of these classes for a member from another class. Such substituted residues can be introduced, for example, into regions of a human antibody that are homologous with non-human antibodies, or into the non-homologous regions of the molecule.

In making changes to the polypeptide or antibody described herein, according to certain embodiments, the hydropathic index of amino acids can be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al. J. Mol. Biol., 157:105-131 (1982). It is known that certain amino acids can be substituted for other amino acids having a similar hydropathic index or score and still retain a similar biological activity. In making changes based upon the hydropathic index, in certain embodiments, the substitution of amino acids whose hydropathic indices are within ±2 is included. In certain embodiments, those which are within ±1 are included, and in certain embodiments, those within ±0.5 are included.

It is also understood in the art that the substitution of like amino acids can be made effectively on the basis of hydrophilicity, particularly where the biologically functional protein or peptide thereby created is intended for use in immunological embodiments, as in the present case. In certain embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0±1); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within ±2 is included, in certain embodiments, those which are within ±1 are included, and in certain embodiments, those within ±0.5 are included. One can also identify epitopes from primary amino acid sequences on the basis of hydrophilicity. These regions are also referred to as “epitopic core regions”.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain embodiments of the variant VH and VL sequences provided above, each HVR either is unaltered, or contains no more than one, two or three amino acid substitutions.

Amino acid sequence insertions include amino- and/or carboxyl-terminal fusions ranging in length from one residue to polypeptides comprising a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

Any cysteine residue not involved in maintaining the proper conformation of the antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment, such as an Fv fragment).

### (ii) Glycosylation Variants

In some embodiments of any of the antibodies provided herein, the antibody is altered to increase or decrease the extent to which the antibody is glycosylated. Addition or deletion of glycosylation sites to an antibody may be conveniently accomplished by altering the amino acid sequence such that one or more glycosylation sites is created or removed.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Where the antibody comprises an Fc region, the carbohydrate attached thereto may be altered. Native antibodies produced by mammalian cells typically comprise a branched, biantennary oligosaccharide that is generally attached by an N-linkage to Asn297 according to Kabat numbering of the CH2 domain of the Fc region. The oligosaccharide may include various carbohydrates, for example, mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. See, e.g., US Patent Publication Nos. 2003/0157108 and 2004/0093621. Examples of publications related to “defucosylated” or “fucose-deficient” antibody variants include: US 2003/0157108; US 2003/0115614; US 2002/0164328; US 2004/0093621; US 2004/0132140; US 2004/0110704; US 2004/0110282; US 2004/0109865; Okazaki et al. J. Mol. Biol. 336:1239-1249 (2004); Yamane-Ohnuki et al. Biotech. Bioeng. 87:614 (2004). Examples of cell lines capable of producing defucosylated antibodies include Led 3 CHO cells deficient in protein fucosylation (Ripka et al. Arch. Biochem. Biophys. 249:533-545 (1986); US 2003/0157108), and knockout cell lines, such as alpha-1,6-fucosyltransferase gene, FUT8, knockout CHO cells (see. e.g., Yamane-Ohnuki et al. Biotech. Bioeng. 87: 614 (2004) and Kanda et al. Biotechnol. Bioeng. 94(4):680-688 (2006)).

(iii) Modified Constant Regions

In some embodiments of any of the antibodies provided herein, the antibody Fc is an antibody Fc isotypes and/or modifications. In some embodiments, the antibody Fc isotype and/or modification is capable of binding to Fc gamma receptor.

In some embodiments of any of the antibodies provided herein, the modified antibody Fc is an IgG1 modified Fc. In some embodiments, the IgG1 modified Fc comprises one or more modifications. For example, in some embodiments, the IgG1 modified Fc comprises one or more amino acid substitutions (e.g., relative to a wild-type Fc region of the same isotype). In some embodiments, the one or more amino acid substitutions are selected from N297A (Bolt S et al. (1993) Eur J Immunol 23:403-411), D265A (Shields et al. (2001) R. J. Biol. Chem. 276, 6591-6604), L234A, L235A (Hutchins et al. (1995) Proc Natl Acad Sci USA, 92:11980-11984; Alegre et al., (1994) Transplantation 57:1537-1543. 31; Xu et al., (2000) Cell Immunol, 200:16-26), G237A (Alegre et al. (1994) Transplantation 57:1537-1543. 31; Xu et al. (2000) Cell Immunol, 200:16-26), C226S, C229S, E233P, L234V, L234F, L235E (McEarchern et al., (2007) Blood, 109:1185-1192), P331S (Sazinsky et al., (2008) Proc Natl Acad Sci USA 2008, 105:20167-20172), S267E, L328F, A330L, M252Y, S254T, and/or T256E, where the amino acid position is according to the EU numbering convention.

In some embodiments of any of the IgG1 modified Fc, the Fc comprises N297A mutation according to EU numbering. In some embodiments of any of the IgG1 modified Fc, the Fc comprises D265A and N297A mutations according to EU numbering. In some embodiments of any of the IgG1 modified Fc, the Fc comprises D270A mutations according to EU numbering. In some embodiments, the IgG1 modified Fc comprises L234A and L235A mutations according to EU numbering. In some embodiments of any of the IgG1 modified Fc, the Fc comprises L234A and G237A mutations according to EU numbering. In some embodiments of any of the IgG1 modified Fc, the Fc comprises L234A, L235A and G237A mutations according to EU numbering. In some embodiments of any of the IgG1 modified Fc, the Fc comprises one or more (including all) of P238D, L328E, E233, G237D, H268D, P271G and A330R mutations according to EU numbering. In some embodiments of any of the IgG1 modified Fc, the Fc comprises one or more of S267E/L328F mutations according to EU numbering. In some embodiments of any of the IgG1 modified Fc, the Fc comprises P238D, L328E, E233D, G237D, H268D, P271G and A330R mutations according to EU numbering. In some embodiments of any of the IgG1 modified Fc, the Fc comprises P238D, L328E, G237D, H268D, P271G and A330R mutations according to EU numbering. In some embodiments of any of the IgG1 modified Fc, the Fc comprises P238D, S267E, L328E, E233D, G237D, H268D, P271G and A330R mutations according to EU numbering. In some embodiments of any of the IgG1 modified Fc, the Fc comprises P238D, S267E, L328E, G237D, H268D, P271G and A330R mutations according to EU numbering. In some embodiments of any of the IgG1 modified Fc, the Fc comprises C226S, C229S, E233P, L234V, and L235A mutations according to EU numbering. In some embodiments of any of the IgG1 modified Fc, the Fc comprises L234F, L235E, and P331S mutations according to EU numbering. In some embodiments of any of the IgG1 modified Fc, the Fc comprises S267E and L328F mutations according to EU numbering. In some embodiments of any of the IgG1 modified Fc, the Fc comprises S267E mutations according to EU numbering. In some embodiments of any of the IgG1 modified Fc, the Fc comprises a substitute of the constant heavy 1 (CH1) and hinge region of IgG1 with CH1 and hinge region of IgG2 (amino acids 118-230 of IgG2 according to EU numbering) with a Kappa light chain.

In some embodiments of any of the IgG1 modified Fc, the Fc includes two or more amino acid substitutions that increase antibody clustering without activating complement as compared to a corresponding antibody having an Fc region that does not include the two or more amino acid substitutions. Accordingly, in some embodiments of any of the IgG1 modified Fc, the IgG1 modified Fc is an antibody comprising an Fc region, where the antibody comprises an amino acid substitution at position E430G and one or more amino acid substitutions in the Fc region at a residue position selected from: L234F, L235A, L235E, S267E, K322A, L328F, A330S, P331S, and any combination thereof according to EU numbering. In some embodiments, the IgG1 modified Fc comprises an amino acid substitution at positions E430G, L243A, L235A, and P331S according to EU numbering. In some embodiments, the IgG1 modified Fc comprises an amino acid substitution at positions E430G and P331S according to EU numbering. In some embodiments, the IgG1 modified Fc comprises an amino acid substitution at positions E430G and K322A according to EU numbering. In some embodiments, the IgG1 modified Fc comprises an amino acid substitution at positions E430G, A330S, and P331S according to EU numbering. In some embodiments, the IgG1 modified Fc comprises an amino acid substitution at positions E430G, K322A, A330S, and P331S according to EU numbering. In some embodiments, the IgG1 modified Fc comprises an amino acid substitution at positions E430G, K322A, and A330S according to EU numbering. In some embodiments, the IgG1 modified Fc comprises an amino acid substitution at positions E430G, K322A, and P331S according to EU numbering.

In some embodiments of any of the IgG1 modified Fc, the IgG1 modified Fc may further comprise herein may be combined with an A330L mutation (Lazar et al. Proc Natl Acad Sci USA, 103:4005-4010 (2006)), or one or more of L234F, L235E, and/or P331S mutations (Sazinsky et al. Proc Natl Acad Sci USA, 105:20167-20172 (2008)), according to the EU numbering convention, to eliminate complement activation. In some embodiments of any of the IgG1 modified Fc, the IgG1 modified Fc may further comprise one or more of A330L, A330S, L234F, L235E, and/or P331S according to EU numbering. In some embodiments of any of the IgG1 modified Fc, the IgG1 modified Fc may further comprise one or more mutations to enhance the antibody half-life in human serum (e.g., one or more (including all) of M252Y, S254T, and T256E mutations according to the EU numbering convention). In some embodiments of any of the IgG1 modified Fc, the IgG1 modified Fc may further comprise one or more of E430G, E430S, E430F, E430T, E345K, E345Q, E345R, E345Y, S440Y, and/or S440W according to EU numbering.

Other aspects of the present disclosure relate to antibodies having modified constant regions (i.e., Fc regions). An antibody dependent on binding to FcgR receptor to activate targeted receptors may lose its agonist activity if engineered to eliminate FcgR binding (see, e.g., Wilson et al. Cancer Cell 19:101-113 (2011); Armour at al. Immunology 40:585-593 (2003); and White et al. Cancer Cell 27:138-148 (2015)). As such, it is thought that an anti-TMEM106B antibody of the present disclosure with the correct epitope specificity can activate the target antigen, with minimal adverse effects, when the antibody has an Fc domain from a human IgG2 isotype (CH1 and hinge region) or another type of Fc domain that is capable of preferentially binding the inhibitory FcgRIIB r receptors, or a variation thereof.

In some embodiments of any of the antibodies provided herein, the modified antibody Fc is an IgG2 modified Fc. In some embodiments, the IgG2 modified Fc comprises one or more modifications. For example, in some embodiments, the IgG2 modified Fc comprises one or more amino acid substitutions (e.g., relative to a wild-type Fc region of the same isotype). In some embodiments of any of the IgG2 modified Fc, the one or more amino acid substitutions are selected from V234A (Alegre et al. Transplantation 57:1537-1543 (1994); Xu et al. Cell Immunol, 200:16-26 (2000)); G237A (Cole et al. Transplantation, 68:563-571 (1999)); H268Q, V309L, A330S, P331S (US 2007/0148167; Armour et al. Eur J Immunol 29: 2613-2624 (1999); Armour et al. The Haematology Journal 1(Suppl. 1):27 (2000); Armour et al. The Haematology Journal 1(Suppl. 1):27 (2000)), C219S, and/or C220S (White et al. Cancer Cell 27, 138-148 (2015)); S267E, L328F (Chu et al. Mol Immunol, 45:3926-3933 (2008)); and M252Y, S254T, and/or T256E according to the EU numbering convention. In some embodiments of any of the IgG2 modified Fc, the Fc comprises an amino acid substitution at positions V234A and G237A according to EU numbering. In some embodiments of any of the IgG2 modified Fc, the Fc comprises an amino acid substitution at positions C219S or C220S according to EU numbering. In some embodiments of any of the IgG2 modified Fc, the Fc comprises an amino acid substitution at positions A330S and P331S according to EU numbering. In some embodiments of any of the IgG2 modified Fc, the Fc comprises an amino acid substitution at positions S267E and L328F according to EU numbering.

In some embodiments of any of the IgG2 modified Fc, the Fc comprises a C127S amino acid substitution according to the EU numbering convention (White et al., (2015) Cancer Cell 27, 138-148; Lightle et al. Protein Sci. 19:753-762 (2010); and WO 2008/079246). In some embodiments of any of the IgG2 modified Fc, the antibody has an IgG2 isotype with a Kappa light chain constant domain that comprises a C214S amino acid substitution according to the EU numbering convention (White et al. Cancer Cell 27:138-148 (2015); Lightle et al. Protein Sci. 19:753-762 (2010); and WO 2008/079246).

In some embodiments of any of the IgG2 modified Fc, the Fc comprises a C220S amino acid substitution according to the EU numbering convention. In some embodiments of any of the IgG2 modified Fc, the antibody has an IgG2 isotype with a Kappa light chain constant domain that comprises a C214S amino acid substitution according to the EU numbering convention.

In some embodiments of any of the IgG2 modified Fc, the Fc comprises a C219S amino acid substitution according to the EU numbering convention. In some embodiments of any of the IgG2 modified Fc, the antibody has an IgG2 isotype with a Kappa light chain constant domain that comprises a C214S amino acid substitution according to the EU numbering convention.

In some embodiments of any of the IgG2 modified Fc, the Fc includes an IgG2 isotype heavy chain constant domain 1(CH1) and hinge region (White et al. Cancer Cell 27:138-148 (2015)). In certain embodiments of any of the IgG2 modified Fc, the IgG2 isotype CH1 and hinge region comprise the amino acid sequence of 118-230 according to EU numbering. In some embodiments of any of the IgG2 modified Fc, the antibody Fc region comprises a S267E amino acid substitution, a L328F amino acid substitution, or both, and/or a N297A or N297Q amino acid substitution according to the EU numbering convention.

In some embodiments of any of the IgG2 modified Fc, the Fc further comprises one or more amino acid substitution at positions E430G, E430S, E430F, E430T, E345K, E345Q, E345R, E345Y, S440Y, and S440W according to EU numbering. In some embodiments of any of the IgG2 modified Fc, the Fc may further comprise one or more mutations to enhance the antibody half-life in human serum (e.g., one or more (including all) of M252Y, S254T, and T256E mutations according to the EU numbering convention). In some embodiments of any of the IgG2 modified Fc, the Fc may further comprise A330S and P331S.

In some embodiments of any of the IgG2 modified Fc, the Fc is an IgG2/4 hybrid Fc. In some embodiments, the IgG2/4 hybrid Fc comprises IgG2 aa 118 to 260 and IgG4 aa 261 to 447. In some embodiments of any IgG2 modified Fc, the Fc comprises one or more amino acid substitutions at positions H268Q, V309L, A330S, and P331S according to EU numbering.

In some embodiments of any of the IgG1 and/or IgG2 modified Fc, the Fc comprises one or more additional amino acid substitutions selected from A330L, L234F; L235E, or P331S according to EU numbering; and any combination thereof.

In certain embodiments of any of the IgG1 and/or IgG2 modified Fc, the Fc comprises one or more amino acid substitutions at a residue position selected from C127S, L234A, L234F, L235A, L235E, S267E, K322A, L328F, A330S, P331S, E345R, E430G, S440Y, and any combination thereof according to EU numbering. In some embodiments of any of the IgG1 and/or IgG2 modified Fc, the Fc comprises an amino acid substitution at positions E430G, L243A, L235A, and P331S according to EU numbering. In some embodiments of any of the IgG1 and/or IgG2 modified Fc, the Fc comprises an amino acid substitution at positions E430G and P331S according to EU numbering. In some embodiments of any of the IgG1 and/or IgG2 modified Fc, the Fc comprises an amino acid substitution at positions E430G and K322A according to EU numbering. In some embodiments of any of the IgG1 and/or IgG2 modified Fc, the Fc comprises an amino acid substitution at positions E430G, A330S, and P331S according to EU numbering. In some embodiments of any of the IgG1 and/or IgG2 modified Fc, the Fc comprises an amino acid substitution at positions E430G, K322A, A330S, and P331S according to EU numbering. In some embodiments of any of the IgG1 and/or IgG2 modified Fc, the Fc comprises an amino acid substitution at positions E430G, K322A, and A330S according to EU numbering. In some embodiments of any of the IgG1 and/or IgG2 modified Fc, the Fc comprises an amino acid substitution at positions E430G, K322A, and P331S according to EU numbering. In some embodiments of any of the IgG1 and/or IgG2 modified Fc, the Fc comprises an amino acid substitution at positions S267E and L328F according to EU numbering. In some embodiments of any of the IgG1 and/or IgG2 modified Fc, the Fc comprises an amino acid substitution at position C127S according to EU numbering. In some embodiments of any of the IgG1 and/or IgG2 modified Fc, the Fc comprises an amino acid substitution at positions E345R, E430G and S440Y according to EU numbering.

In some embodiments of any of the antibodies provided herein, the modified antibody Fc is an IgG4 modified Fc. In some embodiments, the IgG4 modified Fc comprises one or more modifications. For example, in some embodiments, the IgG4 modified Fc comprises one or more amino acid substitutions (e.g., relative to a wild-type Fc region of the same isotype). In some embodiments of any of the IgG4 modified Fc, the one or more amino acid substitutions are selected from L235A, G237A, S229P, L236E (Reddy et al. J Immunol 164:1925-1933(2000)), S267E, E318A, L328F, M252Y, S254T, and/or T256E according to the EU numbering convention. In some embodiments of any of the IgG4 modified Fc, the Fc may further comprise L235A, G237A, and E318A according to the EU numbering convention. In some embodiments of any of the IgG4 modified Fc, the Fc may further comprise S228P and L235E according to the EU numbering convention. In some embodiments of any of the IgG4 modified Fc, the IgG4 modified Fc may further comprise S267E and L328F according to the EU numbering convention.

In some embodiments of any of the IgG4 modified Fc, the IgG4 modified Fc comprises may be combined with an S228P mutation according to the EU numbering convention (Angal et al. Mol Immunol. 30:105-108 (1993)) and/or with one or more mutations described in (Peters et al. J Biol Chem. 287(29):24525-33 (2012)) to enhance antibody stabilization.

In some embodiments of any of the IgG4 modified Fc, the IgG4 modified Fc may further comprise one or more mutations to enhance the antibody half-life in human serum (e.g., one or more (including all) of M252Y, S254T, and T256E mutations according to the EU numbering convention).

In some embodiments of any of the IgG4 modified Fc, the Fc comprises L235E according to EU numbering. In certain embodiments of any of the IgG4 modified Fc, the Fc comprises one or more amino acid substitutions at a residue position selected from C127S, F234A, L235A, L235E, S267E, K322A, L328F, E345R, E430G, S440Y, and any combination thereof, according to EU numbering. In some embodiments of any of the IgG4 modified Fc, the Fc comprises an amino acid substitution at positions E430G, L243A, L235A, and P331S according to EU numbering. In some embodiments of any of the IgG4 modified Fc, the Fc comprises an amino acid substitution at positions E430G and P331S according to EU numbering. In some embodiments of any of the IgG4 modified Fc, the Fc comprises an amino acid substitution at positions E430G and K322A according to EU numbering. In some embodiments of any of the IgG4 modified Fc, the Fc comprises an amino acid substitution at position E430 according to EU numbering. In some embodiments of any of the IgG4 modified Fc, the Fc region comprises an amino acid substitution at positions E430G and K322A according to EU numbering. In some embodiments of any of the IgG4 modified Fc, the Fc comprises an amino acid substitution at positions S267E and L328F according to EU numbering. In some embodiments of any of the IgG4 modified Fc, the Fc comprises an amino acid substitution at position C127S according to EU numbering. In some embodiments of any of the IgG4 modified Fc, the Fc comprises an amino acid substitution at positions E345R, E430G and S440Y according to EU numbering.

### (8) Other Antibody Modifications

In some embodiments of any of the antibodies, the antibody is a derivative. The term “derivative” refers to a molecule that includes a chemical modification other than an insertion, deletion, or substitution of amino acids (or nucleic acids). In certain embodiments, derivatives comprise covalent modifications, including, but not limited to, chemical bonding with polymers, lipids, or other organic or inorganic moieties. In certain embodiments, a chemically modified antigen binding protein can have a greater circulating half-life than an antigen binding protein that is not chemically modified. In certain embodiments, a chemically modified antigen binding protein can have improved targeting capacity for desired cells, tissues, and/or organs. In some embodiments, a derivative antigen binding protein is covalently modified to include one or more water soluble polymer attachments, including, but not limited to, polyethylene glycol, polyoxyethylene glycol, or polypropylene glycol. See. e.g., U.S. Pat. Nos. 4,640,835, 4,496,689, 4,301,144, 4,670,417, 4,791,192 and 4,179,337. In certain embodiments, a derivative antigen binding protein comprises one or more polymer, including, but not limited to, monomethoxy-polyethylene glycol, dextran, cellulose, copolymers of ethylene glycol/propylene glycol, carboxymethylcellulose, polyvinyl pyrrolidone, poly-1, 3-dioxolane, poly-1,3,6-trioxane, ethylene/maleic anhydride copolymer, polyaminoacids (either homopolymers or random copolymers), poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of such polymers.

In certain embodiments, a derivative is covalently modified with polyethylene glycol (PEG) subunits. In certain embodiments, one or more water-soluble polymer is bonded at one or more specific position, for example at the amino terminus, of a derivative. In certain embodiments, one or more water-soluble polymer is randomly attached to one or more side chains of a derivative. In certain embodiments, PEG is used to improve the therapeutic capacity for an antigen binding protein. In certain embodiments, PEG is used to improve the therapeutic capacity for a humanized antibody. Certain such methods are discussed, for example, in U.S. Pat. No. 6,133,426, which is hereby incorporated by reference for any purpose.

Peptide analogs are commonly used in the pharmaceutical industry as non-peptide drugs with properties analogous to those of the template peptide. These types of non-peptide compound are termed “peptide mimetics” or “peptidomimetics.” Fauchere, J. Adv. Drug Res., 15:29 (1986); and Evans et al. J. Med. Chem., 30:1229 (1987), which are incorporated herein by reference for any purpose. Such compounds are often developed with the aid of computerized molecular modeling. Peptide mimetics that are structurally similar to therapeutically useful peptides can be used to produce a similar therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from: —CH₂NH—, —CH₂S—, —CH₂—CH₂—, —CH═CH-(cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) can be used in certain embodiments to generate more stable peptides. In addition, constrained peptides comprising a consensus sequence or a substantially identical consensus sequence variation can be generated by methods known in the art (Rizo and Gierasch Ann. Rev. Biochem., 61:387 (1992), incorporated herein by reference for any purpose); for example, by adding internal cysteine residues capable of forming intramolecular disulfide bridges which cyclize the peptide.

Drug conjugation involves coupling of a biological active cytotoxic (anticancer) payload or drug to an antibody that specifically targets a certain tumor marker (e.g. a polypeptide that, ideally, is only to be found in or on tumor cells). Antibodies track these proteins down in the body and attach themselves to the surface of cancer cells. The biochemical reaction between the antibody and the target protein (antigen) triggers a signal in the tumor cell, which then absorbs or internalizes the antibody together with the cytotoxin. After the ADC is internalized, the cytotoxic drug is released and kills the cancer. Due to this targeting, ideally the drug has lower side effects and gives a wider therapeutic window than other chemotherapeutic agents. Technics to conjugate antibodies are disclosed are known in the art (see, e.g., Jane de Lartigue OncLive Jul. 5, 2012; ADC Review on antibody-drug conjugates; and Ducry et al. Bioconjugate Chemistry 21 (1):5-13 (2010).

### Nucleic Acids, Vectors, and Host Cells

Anti-TMEM106B antibodies of the present disclosure may be produced using recombinant methods and compositions, e.g., as described in U.S. Pat. No. 4,816,567. In some embodiments, isolated nucleic acids having a nucleotide sequence encoding any of the anti-TMEM106B antibodies of the present disclosure are provided. Such nucleic acids may encode an amino acid sequence comprising the V_{L} and/or an amino acid sequence comprising the V_{H} of the anti-TMEM106B antibody (e.g., the light and/or heavy chains of the antibody). In some embodiments, one or more vectors (e.g., expression vectors) comprising such nucleic acids are provided. In some embodiments, a host cell comprising such nucleic acid is also provided. In some embodiments, the host cell comprises (e.g., has been transduced with): (1) a vector comprising a nucleic acid that encodes an amino acid sequence comprising the V_{L} of the antibody and an amino acid sequence comprising the V_{H} of the antibody, or (2) a first vector comprising a nucleic acid that encodes an amino acid sequence comprising the V_{L} of the antibody and a second vector comprising a nucleic acid that encodes an amino acid sequence comprising the V_{H} of the antibody. In some embodiments, the host cell is eukaryotic, e.g., a Chinese Hamster Ovary (CHO) cell or lymphoid cell (e.g., Y0, NS0, Sp20 cell). Host cells of the present disclosure also include, without limitation, isolated cells, in vitro cultured cells, and ex vivo cultured cells.

Methods of making an anti-TMEM106B antibody of the present disclosure are provided. In some embodiments, the method includes culturing a host cell of the present disclosure comprising a nucleic acid encoding the anti-TMEM106B antibody, under conditions suitable for expression of the antibody. In some embodiments, the antibody is subsequently recovered from the host cell (or host cell culture medium).

For recombinant production of an anti-TMEM106B antibody of the present disclosure, a nucleic acid encoding the anti-TMEM106B antibody is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable vectors comprising a nucleic acid sequence encoding any of the anti-TMEM106B antibodies of the present disclosure, or cell-surface expressed fragments or polypeptides thereof polypeptides (including antibodies) described herein include, without limitation, cloning vectors and expression vectors. Suitable cloning vectors can be constructed according to standard techniques, or may be selected from a large number of cloning vectors available in the art. While the cloning vector selected may vary according to the host cell intended to be used, useful cloning vectors generally have the ability to self-replicate, may possess a single target for a particular restriction endonuclease, and/or may carry genes for a marker that can be used in selecting clones comprising the vector. Suitable examples include plasmids and bacterial viruses, e.g., pUC18, pUC19, Bluescript (e.g., pBS SK+) and its derivatives, mpl8, mpl9, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Suitable host cells for cloning or expression of antibody-encoding vectors include prokaryotic or eukaryotic cells. For example, anti-TMEM106B antibodies of the present disclosure may be produced in bacteria, in particular when glycosylation and Fc effector function are not needed. For expression of antibody fragments and polypeptides in bacteria (e.g., U.S. Pat. Nos. 5,648,237, 5,789,199, and 5,840,523. After expression, the antibody may be isolated from the bacterial cell paste in a soluble fraction and can be further purified.

In addition to prokaryotes, eukaryotic microorganisms, such as filamentous fungi or yeast, are also suitable cloning or expression hosts for antibody-encoding vectors, including fungi and yeast strains whose glycosylation pathways have been “humanized,” resulting in the production of an antibody with a partially or fully human glycosylation pattern (e.g., Gemgross Nat. Biotech. 22:1409-1414 (2004); and Li et al. Nat. Biotech. 24:210-215 (2006)).

Suitable host cells for the expression of glycosylated antibody can also be derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts (e.g., U.S. Pat. Nos. 5,959,177, 6,040,498, 6,420,548, 7,125,978, and 6,417,429, describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al. J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al. Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al. Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B.K.C. Lo, ed., Humana Press, Totowa, NJ), pp. 255-268 (2003).

### Pharmaceutical Compositions/Formulations

Provided herein are pharmaceutical compositions and/or pharmaceutical formulations comprising the anti-TMEM106B antibodies of the present disclosure and a pharmaceutically acceptable carrier.

In some aspects, the antibody or antigen-binding fragment thereof having the desired degree of purity is present in a formulation comprising, e.g., a physiologically acceptable carrier, excipient or stabilizer (Remington's Pharmaceutical Sciences (1990) Mack Publishing Co., Easton, PA). In some embodiments, pharmaceutically acceptable carriers preferably are nontoxic to recipients at the dosages and concentrations employed.

Formulations suitable for parenteral administration include aqueous and non-aqueous, isotonic sterile injection solutions, which can comprise antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

In some aspects, a pharmaceutical composition comprises an anti-TMEM106B antibody or antigen-binding fragment thereof as described herein, and a pharmaceutically acceptable carrier (see, e.g., Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)). Pharmaceutical compositions described herein are, in some aspects, for use as a medicament. The compositions to be used for in vivo administration can be sterile. This is readily accomplished by filtration through, e.g., sterile filtration membranes.

A pharmaceutical composition described herein can be used to exert a biological effect(s) in vivo or in vitro.

### Therapeutic Uses

As disclosed herein, anti-TMEM106B antibodies of the present disclosure may be used for preventing, reducing risk, or treating various diseases, disorders, and conditions. In some embodiments, an anti-TMEM106B antibody of the present disclosure is effective at preventing, reducing risk, or treating frontotemporal lobar degeneration, frontotemporal dementia, frontotemporal dementia with progranulin mutations, frontotemporal dementia with C90rf72 mutations, frontotemporal lobar degeneration with TDP-43 inclusions, TDP-43 proteinopathy, hippocampal sclerosis (HpScl), hippocampal sclerosis of aging (HS-Aging), cognitive impairments associated with various disorders (including without limitation cognitive impairment in amyotrophic lateral sclerosis), cognitive impairments associated with chronic traumatic encephalopathy, and hypomyelinating disorders (including without limitation hypomyelinating leukodystrophy).

Other aspects of the present disclosure relate to a method of preventing, reducing risk, or treating an individual having a disease, disorder, or injury selected from the group consisting of frontotemporal dementia, Alzheimer's disease, vascular dementia, seizures, retinal dystrophy, a traumatic brain injury, a spinal cord injury, long-term depression, atherosclerotic vascular diseases, undesirable symptoms of normal aging, dementia, mixed dementia, Creutzfeldt-Jakob disease, normal pressure hydrocephalus, amyotrophic lateral sclerosis, Huntington's disease, taupathy disease, stroke, acute trauma, chronic trauma, lupus, acute and chronic colitis, Crohn's disease, inflammatory bowel disease, ulcerative colitis, malaria, essential tremor, central nervous system lupus, Behcet's disease, Parkinson's disease, dementia with Lewy bodies, multiple system atrophy, degenerative disc disease, Shy-Drager syndrome, progressive supranuclear palsy, cortical basal ganglionic degeneration, acute disseminated encephalomyelitis, granulomartous disorders, Sarcoidosis, diseases of aging, age related macular degeneration, glaucoma, retinitis pigmentosa, retinal degeneration, respiratory tract infection, sepsis, eye infection, systemic infection, inflammatory disorders, arthritis, multiple sclerosis, metabolic disorder, obesity, insulin resistance, type 2 diabetes, tissue or vascular damage, an injury, and one or more undesirable symptoms of normal aging, comprising administering to the individual a therapeutically effective amount of the anti-TMEM106B antibody of any of the preceding embodiments. In some embodiments, an anti-TMEM106B antibody of the present disclosure may reduce TDP-43 inclusions in brain, reduce decline in cognitive and behavioral function, and improve cognitive and behavioral function. Assessment of a reduction in the decline of cognitive and/or behavioral function or of an improvement in cognitive and/or behavioral function is determined using assessment tools available to one of skill, including Clinical Dementia Rating Sum of Boxes (CDR-SB) score (changes after dosing relative to baseline), Mini-Mental State Examination (MMSE) score (changes after dosing relative to baseline), or Repeatable Battery for the Assessment of Neuropsychological Status (RBANS) score (changes after dosing relative to baseline).

In some embodiments, a subject or individual is a mammal. Mammals include, without limitation, domesticated animals (e.g., cows, sheep, cats, dogs, and horses), primates (e.g., humans and non-human primates such as monkeys), rabbits, and rodents (e.g., mice and rats). In some embodiments, the subject or individual is a human.

An antibody provided herein (and any additional therapeutic agent) can be administered by any suitable means, including parenteral, intrapulmonary, intranasal, intralesional administration, intracerobrospinal, intracranial, intraspinal, intrasynovial, intrathecal, oral, topical, or inhalation routes. Parenteral infusions include intramuscular, intravenous administration as a bolus or by continuous infusion over a period of time, intraarterial, intra-articular, intraperitoneal, or subcutaneous administration. In some embodiments, the administration is intravenous administration. In some embodiments, the administration is subcutaneous. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies provided herein would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody of the invention (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments.

### Diagnostic Uses

In some embodiments of any of the antibodies, any of the anti-TMEM106B antibodies provided herein is useful for detecting the presence of TMEM106B in a sample or an individual. In some embodiments, any of the anti-TMEM106B antibodies provided herein is useful for detecting the presence of TMEM106B in a cell, including detecting the presence of TMEM106B in the lysosomal and/or endosomal compartment of a cell. The term “detecting” as used herein encompasses quantitative or qualitative detection. Provided herein are methods of using the antibodies of this disclosure for diagnostic purposes, such as the detection of TMEM106B in an individual or in tissue samples derived from an individual. In some embodiments, the individual is a human. In some embodiments, the tissue sample is blood, brain, spinal fluid, etc.

The detection method may involve quantification of the antigen-bound antibody. Antibody detection in biological samples may occur with any method known in the art, including immunofluorescence microscopy, immunocytochemistry, immunohistochemistry, ELISA, FACS analysis, immunoprecipitation, or micro-positron emission tomography. In certain embodiments, the antibody is radiolabeled, for example with ¹⁸F and subsequently detected utilizing micro-positron emission tomography analysis. Antibody-binding may also be quantified in a patient by non-invasive techniques such as positron emission tomography (PET), X-ray computed tomography, single-photon emission computed tomography (SPECT), computed tomography (CT), and computed axial tomography (CAT).

### Articles of Manufacture

Provided herein are articles of manufacture (e.g., kit) comprising an anti-TMEM106B antibody described herein. Article of manufacture may include one or more containers comprising an antibody described herein. Containers may be any suitable packaging including, but is not limited to, vials, bottles, jars, flexible packaging (e.g., sealed Mylar or plastic bags), and the like. The containers may be unit doses, bulk packages (e.g., multi-dose packages) or sub-unit doses.

In some embodiments, the kits may further include a second agent. In some embodiments, the second agent is a pharmaceutically-acceptable buffer or diluting agent including, but not limited to, such as bacteriostatic water for injection (BWFI), phosphate-buffered saline, Ringer's solution and dextrose solution. In some embodiments, the second agent is a pharmaceutically active agent.

In some embodiments of any of the articles of manufacture, the article of manufactures further include instructions for use in accordance with the methods of this disclosure. The instructions generally include information as to dosage, dosing schedule, and route of administration for the intended treatment. In some embodiments, these instructions comprise a description of administration of the isolated antibody of the present disclosure (e.g., an anti-TMEM106B antibody described herein) to prevent, reduce risk, or treat an individual having a disease, disorder, or injury selected from frontotemporal lobar degeneration, frontotemporal dementia, frontotemporal dementia with progranulin mutations, frontotemporal dementia with C9orf72 mutations, frontotemporal lobar degeneration with TDP-43 inclusions, TDP-43 proteinopathy, hippocampal sclerosis (HpScl), hippocampal sclerosis of aging (HS-Aging), cognitive impairments associated with various disorders (including without limitation cognitive impairment in amyotrophic lateral sclerosis), and hypomyelinating disorder (including without limitation hypomyelinating leukodystrophy), according to any methods of this disclosure. In some embodiments, the instructions include instructions for use of the anti-TMEM106B antibody and the second agent (e.g., second pharmaceutically active agent).

The present disclosure will be more fully understood by reference to the following Examples. They should not, however, be construed as limiting the scope of the present disclosure. All citations throughout the disclosure are hereby expressly incorporated by reference.

### EXAMPLES

### Example 1: Production of GST and Murine-Fc-Conjugated Human TMEM106B

Various human TMEM106B polypeptides and TMEM106B polypeptide fusion proteins were generated as follows. Mammalian recombinant expression of various TMEM106B polypeptides was performed by cloning synthetic genes based on TMEM106B cDNA into mammalian expression vectors, followed by transient transfection and expression in HEK293T cells. Each TMEM106B expression construct included a heterologous signal peptide and a Glutathione-S-transferase. TMEM106B expression constructs included the C-terminal region of TMEM106B (the putative extracellular domain (ECD) (amino acid residues 122-274 of SEQ ID NO:1). Other TMEM106B expression constructs included a truncated version of the ECD (amino acid residues 122-210 of SEQ ID NO:1) in order to avoid expression of a hydrophobic patch within the TMEM106B protein (located at approximately amino acid residues 210-240), thought to possibly impair folding of the soluble TMEM106B protein product.

The amino acid sequences of each TMEM106B polypeptide used in the expression constructs are provided below:

```
GST Fusion human TMEM106B ECD (Truncated)
(SEQ ID NO: 266)
    1 MDMRVPAQLL GLLLLWLRGA RCMSPILGYW KIKGLVQPTR LLLEYLEEKY
  51 EEHLYERDEG DKWRNKKFEL GLEFPNLPYY IDGDVKLTQS MAIIRYIADK
101 HNMLGGCPKE RAEISMLEGA VLDIRYGVSR IAYSKDFETL KVDFLSKLPE
151 MLKMFEDRLC HKTYLNGDHV THPDFMLYDA LDVVLYMDPM CLDAFPKLVC
201 FKKRIEAIPQ IDKYLKSSKY IAWPLQGWQA TFGGGDHPPK SDPREFIVTD
251 DIEGRMDPDV KYIGVKSAYV SYDVQKRTIY LNITNTLNIT NNNYYSVEVE
301 NITAQVQFSK TVIGKARLNN ITIIGPLDMK QIDYTVPTVI AEEMSYM**
GST Fusion human TMEM106B ECD
(SEQ ID NO: 267)
    1 MDMRVPAQLL GLLLLWLRGA RCMSPILGYW KIKGLVQPTR LLLEYLEEKY
  51 EEHLYERDEG DKWRNKKFEL GLEFPNLPYY IDGDVKLTQS MAIIRYIADK
101 HNMLGGCPKE RAEISMLEGA VLDIRYGVSR IAYSKDFETL KVDFLSKLPE
151 MLKMFEDRLC HKTYLNGDHV THPDFMLYDA LDVVLYMDPM CLDAFPKLVC
201 FKKRIEAIPQ IDKYLKSSKY IAWPLQGWQA TFGGGDHPPK SDPREFIVTD
251 DIEGRMDPPR SIDVKYIGVK SAYVSYDVQK RTIYLNITNT LNITNNNYYS
301 VEVENITAQV QFSKTVIGKA RLNNITIIGP LDMKQIDYTV PTVIAEEMSY
351 MYDFCTLISI KVHNIVLMMQ VTVTTTYFGH SEQISQERYQ YVDCGRNTTY
401 QLGQSEYLNV LQPQQ**
```

These constructs were used in transient transfection experiments described below for antibody binning characterization.

### Example 2: Construction of TMEM106B Expression Plasmids for DNA Immunization

A DNA immunization approach was used for developing antibodies directed against TMEM106B. cDNA sequences encoding human TMEM106B, mouse TMEM106B, and cynomolgus (cyno) TMEM106B (SEQ ID NOs:1, 2 and 3, respectively) were cloned into the pCAGGS mammalian expression vector (KeraFAST EH1017) for DNA immunization. Expression of each TMEM106B polypeptide was confirmed by transient transfection of the expression constructs into HEK293T cells, followed by Western blot and intracellular and extracellular FACS analysis using commercial-available anti-TMEM106B antibodies (EMD Millipore MAB-N473, Thermo-Fischer PA5-6338, Abcam ab140185, Abcam abi16023, Protein Tech 20995-1-AP, LifeSpan Biosciences LS-C145601, Abgent A112796, MyBioSource MBS9412982, Sigma SAB2106773, Bethyl Labs A303-439A). The expression constructs where then used for DNA immunization in mice as described below.

### Example 3: Generation of Anti-TMEM106B Hybridoma Antibodies

In order to obtain antibodies against TMEM106B, the following procedures were used to generate hybridomas. NZB/W mice (JAX100008, Jackson Laboratory, Bar Harbor, ME), SJL mice (JAX000686, Jackson Laboratory), or TMEM106B.knockout mice (Taconic, Rensselaer, NY) were co-immunized weekly with 50 μg each of plasmid DNA encoding full-length human, cyno, or mouse TMEM106B (SEQ ID NOs:1, 2, and 3) with or without mFlt3 ligand (DNA) and mGM-CSF (DNA) (Invitrogen, San Diego, CA) diluted in lactated Ringer's solution. A total of 5-7 injections of the TMEM106B expression plasmids for DNA immunizations were performed per mouse. Spleens were harvested from the mice three days following the final DNA immunization. Sera from the mice were analyzed for reactivity to TMEM106B by FACS analyses using HEK293 cells overexpressing human, cyno, and/or mouse TMEM106B. Splenocytes from mice whose sera demonstrated strong binding to HEK293 cells overexpressing human, cyno, and/or mouse TMEM106B were fused with P3X63Ag8.653 mouse myeloma cells (CRL-1580, American Type Culture Collection, Rockville, MD) via electrofusion (ECM 2001, BTX, Holliston, MA) and incubated at 37° C./5% CO2 overnight in Clonacell-HY Medium C (StemCell Technologies, Vancouver, BC, Canada). Three rounds of fusions were performed: Fusion A, using splenocytes obtained from immunized TMEM106B.knockout mice; Fusion B, using splenocytes obtained from immunized SJL mice; and Fusion C, using splenocytes obtained from immunized NZB/W mice.

The following day, the fused cells were centrifuged and resuspended in 10 mls of ClonaCell-HY Medium C with anti-mouse IgG Fc-FITC (Jackson Immunoresearch, West Grove, PA) and then gently mixed with 90 mls of methylcellulose-based ClonaCell-HY Medium D (Stemcell Technologies) containing HAT components. The cells were plated into Nunc OmniTrays (Thermo Fisher Scientific, Rochester, NY) and allowed to grow at 37° C./5% CO2 for seven days. Fluorescent colonies were selected and transferred into 96-well plates containing Clonacell-HY Medium E (StemCell Technologies) using a Clonepix 2 (Molecular Devices, Sunnyvale, CA) and screened for TMEM106B reactivity 5 days later.

### Example 4: Primary Screening of Anti-TMEM106B Hybridomas

Initial screening of the anti-TMEM106B hybridomas was performed as follows. Tissue culture supernatants from the hybridomas obtained were initially screened for their ability to differentially bind human TMEM106B transiently-transfected HEK293 cells by comparing the extent of binding to parental (non-transfected) HEK293 cells compared to transfected cells. TMEM106B over-expressing cells were produced via transient transfection of HEK293 cells using the lipofectamine system, according to the manufacturer's protocol with modifications as described above in Example 1. To ensure reproducibility across screening experiments, a large bank of transfected cells (−1×10⁹) was prepared in a single round of transient transfection, and aliquoted and frozen for all further screening experiments.

For screening of the hybridoma cell culture supernatants, humanTMEM106B-transfected HEK293 cells were aliquoted in 96-well round bottom plates (2×10⁵ cells per well) and incubated with 50 μL of hybridoma cell culture supernatant on ice for 30 minutes. After this primary incubation, the supernatant was removed via centrifugation, the cells were washed twice with 175 μL of ice-cold FACS buffer (PBS+1% FBS+2 mM EDTA), and then further incubated on ice for 20 minutes with anti-mouse IgG Fc-APC (Jackson Labs, Cat #115-136-071) (diluted 1:500). Following this secondary incubation, the cells were again washed twice with ice-cold FACS buffer and resuspended in a final volume of 30 μL of FACS buffer+0.25 μl/well propidium iodide (BD Biosciences Cat #556463). Cell sorting was performed on a FACS Canto system (BD Biosciences) or iQue (Intellicyt), with sort gates drawn to exclude dead (i.e., propidium iodide-positive) cells. Median fluorescence intensity (MFI) of anti-mouse-APC MFI on TMEM106B+ HEK293 cells was calculated for each clone, and those displaying a signal of at least 2-fold over background compared to a secondary-antibody only well were taken forward for further analysis and characterization.

### Example 5: Molecular Cloning of Anti-TMEM106B Hybridoma Antibodies

Anti-TMEM106B antibodies obtained from the hybridomas were subcloned as follows. 5×10⁵ hybridoma cells were resuspended in 0.5 ml Trizol solution (Thermo Fisher Scientific, cat #15596026). Total RNA was extracted from the cells by chloroform extraction and ethanol precipitation. cDNA was generated by using Clontech's SMARTer® RACE 5′/3′ Kit (Takara Bio USA Inc, Cat. No. 634859) following the manufacturer's protocol. Variable heavy and light immunoglobulin regions were cloned separately by touchdown PCR using the 5′ UPM primer provided in the RACE kit and heavy chain constant region primer (5′-AGCTGGGAAGGTGTGCACA-3′) [SEQ ID NO:264] and light constant region primer (5′-CCATITTGTCGTTCACTGCCA-3′) [SEQ ID NO:265]. PCR products were purified by QIAquick PCR Purification Kit (QIAGEN, Cat No. 28106) and ligated into a pCR2.1®-TOPO® cloning vector (TOPO® TA cloning Kit, Invitrogen) and transformed into ONESHOT® TOP10 Competent cells. Transformed Escherichia coli (E. coli) colonies were isolated and the variable heavy chain (VH) and variable light chain (VL) nucleic acids were sequenced for each corresponding hybridoma cell line. Following the sequence determination, variable heavy chain regions and variable light chain regions were amplified by PCR using primers containing endonuclease restriction sites (BsrGI and BstEII for HV and BssHII and BsiWI for LV) and subcloned into pJG mammalian expression vector (Alector Inc.) encoding human IgG1 and IgGK, respectively.

### Example 6: Production of Recombinant Anti-TMEM106B Antibodies

Purified hybridoma-derived anti-TMEM106B antibodies were purified using Protein A from hybridoma supernatants after culturing the hybridomas in low-IgG or chemically defined media. Some of the anti-TMEM106B antibodies were also produced via direct cloning of the variable gene regions obtained from the hybridomas into a recombinant expression plasmid for production of chimeric antibodies containing a human Fc domain (human IgG1). The expression plasmids were transiently transfected into Expi293 cells and the resulting anti-TMEM106B antibodies purified via Protein A.

Recombinant production of anti-TMEM106B antibodies was performed as follows. Expression plasmids containing nucleic acid encoding the anti-TMEM106B antibody VH and VL chains used for recombinant antibody expression in Expi293 cells. Transfection of expression plasmids was carried out using the Expifectamine-293 system (ThermoFischerScientific Cat #A 14524) according to the manufacturer's protocol. Briefly, for each anti-TMEM106B antibody, 12 μg of light chain plasmid DNA and 18 μg of heavy chain plasmid DNA was diluted into 1.5 mL OptiMEM (ThermoFischerScientific Cat #31985070), to which was added 80 μL of Expifectamine reagent. The resulting solution was mixed and incubated at room temperature for 30 minutes prior to addition to 30 mL of Expi293 cells (ThemoFischerScientific A14527) in Expi293 expression media (ThermoFischerScientific Cat #A1435101) in 125 mL flasks (Fischer Scientific FIS #PBV12-5). The cells were cultured to approximately 3×10≢cells/ml prior to transfection. Culture conditions for Expi293 cells were 37° C./8% CO2 with orbital shaking at 125 rpm. 16-24 hours after transfection, 150 μL of ExpiFectamine™ 293 Transfection Enhancer 1 and 1.5 mL of ExpiFectamine™ 293 Transfection Enhancer 2 were added to each flask to enhance recombinant antibody yield. Culture supernatants were harvested 5-7 days after transfection, filtered (0.2 micron), and purified via Protein A chromatography.

### Example 7: Anti-TMEM106B Antibody Cross-Reactivity

Anti-human TMEM106B antibody positive clones obtained from the initial rounds of sorting as described above were screened for cross-reactivity to mouse TMEM106B and cynomolgus (cyno) TMEM106B using a method similar to that used in the initial screen but using HEK293 cells overexpressing human TMEM106B, murine TMEM106B, or cynomolgus TMEM106B, as well as parental HEK293 cells as a negative control. The anti-TMEM106B antibodies from the hybridoma clones were purified and were identified as human/mouse/cyno cross-reactive, human-only, human/mouse cross-reactive, and human/cyno cross-reactive based on the results of this study. Results of these cell binding studies and associated cross-reactivity screen are shown below in Table 1; the data is presented as fold-change in binding to HEK293 cells transiently transfected with either human TMEM106B (hu), cyno TMEM106B, or murine TMEM106B (mu) over binding to the parental HEK293 cells.

TABLE 1

| Antibody | huTMEM106B | cynoTMEM106B | muTMEM106B |
|---|---|---|---|
| TM-54 | 15.61 | 6.27 | 1.17 |
| TM-56 | 9.35 | 4.49 | 0.47 |
| TM-59 | 8.71 | 4.40 | 0.42 |
| TM-60 | 4.39 | 2.63 | 0.43 |
| TM-61 | 5.07 | 5.15 | 0.68 |
| TM-62 | 11.37 | 2.93 | 0.86 |
| TM-63 | 7.12 | 8.24 | 0.62 |
| TM-64 | 12.81 | 7.12 | 1.07 |
| TM-65 | 4.14 | 2.15 | 1.35 |
| TM-66 | 5.03 | 7.13 | 4.07 |
| TM-70 | 2.40 | 3.34 | 2.15 |
| TM-72 | 3.81 | 1.78 | 0.23 |
| TM-73 | 2.57 | 1.15 | 0.59 |
| TM-74 | 1.80 | 1.19 | 0.39 |
| TM-76 | 6.52 | 4.35 | 0.70 |
| TM-77 | 1.35 | 2.34 | 1.86 |
| TM-78 | 9.60 | 6.88 | 0.99 |
| TM-79 | 3.39 | 2.58 | 1.86 |
| TM-80 | 9.21 | 4.33 | 0.39 |
| TM-82 | 1.99 | 1.49 | 0.88 |
| TM-83 | 0.42 | 0.60 | 9.91 |
| TM-84 | 17.05 | 8.43 | 9.62 |
| TM-85 | 0.26 | 0.18 | 0.19 |
| TM-86 | 9.73 | 5.81 | 5.78 |
| TM-87 | 12.82 | 7.21 | 26.98 |
| TM-88 | 7.81 | 7.57 | 5.84 |
| TM-90 | 1.25 | 1.32 | 1.97 |
| TM-91 | 5.22 | 2.45 | 2.41 |
| TM-92 | 9.14 | 5.70 | 5.15 |
| TM-93 | 11.49 | 9.21 | 8.83 |
| TM-94 | 3.79 | 6.10 | 2.35 |

These results indicated that the isolated anti-TMEM106B antibodies obtained are specific for the TMEM106B protein and are generally cross-reactive to TMEM106B proteins of human, mouse, and cynomolgus origin. In general, the anti-TMEM106B antibodies displayed a high-degree of human and cyno cross-reactivity, as predicted based on the very high homology of these proteins.

### Example 8: Antibody Heavy Chain and Light Chain Variable Domain Sequences

Sequences were determined for the positive hybridoma anti-TMEM106B antibodies identified. Using standard techniques, the amino acid sequences encoding the light chain variable regions and the heavy chain variable regions of the generated antibodies were determined. The Kabat heavy chain CDR (HVR) amino acid sequences and the Kabat light chain CDR (HVR) amino sequences of the antibodies are set forth below in Table 2 and Table 3, respectively. The amino acid sequences for the heavy chain and light chain variable regions are set forth below in Table 4. In Table 4, the CDR (HVR) regions, as defined by Kabat, are underlined.

TABLE 2

| | | SEQ | | SEQ | | SEQ |
|---|---|---|---|---|---|---|
| Anti- | | ID | | ID | | ID |
| body | CDR-H1 | NO: | CDR-H2 | NO: | CDR-H3 | NO: |
| TM-54 | TYDLN | 77 | WIYPRGGSTEY | 107 | YSIYAMDY | 143 |
| | | | NEKFKG | | | |
| TM-56 | SYWMH | 78 | YINPSSGYSKY | 108 | EGGSISDWY | 144 |
| | | | NQRFKD | | FDV | |
| TM-59 | SYWMH | 78 | YVNPSSGYTKN | 109 | EGGSISDWY | 144 |
| | | | NQKFKD | | FDV | |
| TM-60 | SYDIN | 79 | WIYPRDGNTQY | 110 | WIFYAMDY | 145 |
| | | | IEKLKG | | | |
| TM-61 | NYWMN | 80 | YINPSSDYTKY | 111 | WGGSRSYWY | 146 |
| | | | NQNFKD | | FDV | |
| TM-62 | RYWMH | 81 | NIDPSDSETQY | 112 | LTTLDYAMDY | 147 |
| | | | NPKFKD | | | |
| TM-63 | SFWMH | 82 | NIDPSDSETHY | 113 | SSNWDNFDY | 148 |
| | | | SQKFKD | | | |
| TM-64 | SYWMH | 78 | NIDPSDSDTHY | 114 | SRGGTYDY | 149 |
| | | | NQNFRG | | GEAMDY | |
| TM-65 | SSWMH | 83 | EINPKNGGTNY | 115 | PTWFTY | 150 |
| | | | NEKFKT | | | |
| TM-66 | NYAMS | 84 | TIGSVGSYTYY | 116 | QEGSSHFDY | 151 |
| | | | PDSVKG | | | |
| TM-68 | DDYMH | 85 | WIDPENGDTEY | 117 | PQTATTC | 152 |
| | | | ASKFQG | | | |
| TM-69 | SYGVH | 86 | IMGWDDKKYYN | 118 | EAGPYGMDY | 153 |
| | | | SALKS | | | |
| TM-70 | NYAMS | 84 | TIGYGGSYSYY | 119 | QDSNYEGVW | 154 |
| | | | PDSVKG | | FAY | |
| TM-71 | NYAMS | 84 | TIGYGGSYSYY | 119 | QDSNYEGVWF | 154 |
| | | | PDSVKG | | AY | |
| TM-72 | NYWMH | 87 | NIDPSDSDTHY | 120 | SRGGYYDYGE | 155 |
| | | | NQKFKD | | AMDY | |
| TM-73 | SHWMH | 88 | EIDPSDSYTYY | 121 | NYHGSGPPF | 156 |
| | | | NQKFKG | | AH | |
| TM-74 | SYVIH | 89 | FINPNNDGTKY | 122 | PYYDYEGYTM | 157 |
| | | | NEKFRG | | DY | |
| TM-75 | SYVMH | 90 | YINPYNDGTKY | 123 | GGWFPYAMDY | 158 |
| | | | NEKFKG | | | |
| TM-76 | TSGMGVS | 91 | HIFWDDDKRYN | 124 | MGRQRDYFDY | 159 |
| | | | LFLKS | | | |
| TM-77 | DYEMH | 92 | VIDPETGGTAY | 125 | YGIEGY | 160 |
| | | | NQMFKG | | | |
| TM-78 | NHWMH | 93 | NIDPSDSDTHY | 126 | SRGGYYDY | 155 |
| | | | NHKFKD | | GEAMDY | |
| TM-79 | NYVMH | 94 | HITPHNDGTKY | 127 | SGQFGRGDY | 161 |
| | | | NEKFKG | | FHY | |
| TM-80 | TFWMH | 95 | NIDPSDSETHY | 128 | SSNWDNFDY | 148 |
| | | | NQEFKD | | | |
| TM-81 | SYWMH | 78 | NIDPSDSEIHY | 129 | SPAGQILDY | 162 |
| | | | NQKFKD | | | |
| TM-82 | NYLIE | 96 | VINPGSGITDY | 130 | SPFITTVVADY | 163 |
| | | | SEKFKG | | | |
| TM-83 | TYWIH | 97 | NINPSNGNTKN | 131 | GDYYGTSYPF | 164 |
| | | | NERFKT | | | |
| TM-84 | TFPIE | 98 | NFHPYNDDTKY | 132 | YFYGGMDY | 165 |
| | | | NEKFKG | | | |
| TM-85 | GYWMH | 99 | RIDPYSGDTRY | 133 | YGLGHAMDY | 166 |
| | | | NEKFKN | | | |
| TM-86 | DYYIH | 100 | VISPYNDNTNY | 134 | TYYSNYFDY | 167 |
| | | | NQKFKG | | | |
| TM-87 | DYYMN | 101 | VINPYNGATSY | 135 | GNYDWYFDV | 168 |
| | | | NQKFQD | | | |
| TM-88 | DYYIN | 102 | VINPYSGATYY | 136 | GGYDYGHW | 169 |
| | | | TQKFKG | | YFDV | |
| TM-89 | NYLIE | 96 | VINPGSGGTDY | 137 | DPYSKYVHY | 170 |
| | | | NEKFKG | | PMDY | |
| TM-90 | SYDIN | 79 | WIYPRDGNAKY | 138 | VSDYHGGYG | 171 |
| | | | NEKFKG | | MDY | |
| TM-91 | SGYYWD | 103 | YISYDGNNNYN | 139 | AVYSVGFAY | 172 |
| | | | PSLKN | | | |
| TM-92 | DYTMD | 104 | HINPNDGGTFY | 140 | WVYYYGISY | 173 |
| | | | NQKFKG | | EGGAMDY | |
| TM-93 | SYGIS | 105 | VIWTGGGTNYN | 141 | NPPKIFYDY | 174 |
| | | | SALES | | LMYFDY | |
| TM-94 | DYNMD | 106 | DINPYNGGTLY | 142 | VGYHGTPDY | 175 |
| | | | NQKFKG | | | |

TABLE 3

| | | SEQ | | SEQ | | SEQ |
|---|---|---|---|---|---|---|
| Anti- | | ID | | ID | | ID |
| body | CDR-L1 | NO: | CDR-L2 | NO: | CDR-L3 | NO: |
| TM-54 | RSSQSIVHGN | 176 | KVSNRFS | 209 | FQGSHVPWT | 232 |
| | GNTYLE | | | | | |
| TM-56 | RSSKSLLQNN | 177 | RMSNLAS | 210 | MQHLEYPYT | 233 |
| | GNTYLY | | | | | |
| TM-59 | RSSKSLLHSN | 178 | RMSNLAS | 210 | MQHLEYPYT | 233 |
| | GNTYSY | | | | | |
| TM-60 | RSSQSIVHGN | 176 | KVSNRFS | 209 | FQGSHLPYT | 234 |
| | GNTYLE | | | | | |
| TM-61 | KSSQSLLNSG | 179 | GASTRES | 211 | QNDSNYPFT | 235 |
| | NQKNYLA | | | | | |
| TM-62 | SASSSVSYMY | 180 | RTSNLAS | 212 | QQYHSYPPT | 236 |
| TM-63 | RSSQSLLHVN | 181 | RMSNLAS | 210 | MQHLQYPFS | 237 |
| | GNTYLY | | | | | |
| TM-64 | RSSKSLLHTN | 182 | RMSNLAS | 210 | MQHLEYPFT | 238 |
| | GNTYLF | | | | | |
| TM-65 | RASQNINIWL | 183 | KASNLHT | 213 | LQGQSYPYT | 239 |
| | S | | | | | |
| TM-66 | RSSQSLVNSY | 184 | GISNRFS | 214 | LQGTHPPLT | 240 |
| | GKTFLS | | | | | |
| TM-68 | SSSKSLLHSS | 185 | RMSNLAS | 210 | AQMLERPWT | 241 |
| | GITYLY | | | | | |
| TM-69 | RSSQSLVHSN | 186 | KVSNRFS | 209 | SQNTHVPLT | 242 |
| | GNTYLH | | | | | |
| TM-70 | RASESVDNYG | 187 | AASNQGS | 215 | QQSKDVPWT | 243 |
| | ISFMN | | | | | |
| TM-71 | RSSKSLLHTN | 182 | RKSNLAS | 216 | MQHLEYPFT | 238 |
| | GNTYLF | | | | | |
| TM-72 | RSSKSLLHTN | 182 | RKSNLAS | 216 | MQHLEYPFT | 238 |
| | GNTYLF | | | | | |
| TM-73 | SASSGISYIY | 188 | DTSNLAS | 217 | HQWRSYPPT | 244 |
| TM-74 | KASQNVRSAV | 189 | LASNRHT | 218 | LQHWKYPFT | 245 |
| | A | | | | | |
| TM-75 | RASQSVDYNG | 190 | AASNLES | 219 | QQSIEDPWT | 246 |
| | ISYMH | | | | | |
| TM-76 | SASLSISYVH | 191 | GTSNLAS | 220 | HQWSIYRT | 247 |
| TM-77 | KSSQSLFHSN | 192 | MVSKLES | 221 | LQVTHFPLT | 248 |
| | GKTYLN | | | | | |
| TM-78 | RSSKSLLHVN | 193 | RMSNLAS | 210 | MQHLEYPFT | 238 |
| | GNTYLF | | | | | |
| TM-79 | RASKSVSTSG | 194 | LTSNLES | 222 | QHSGELPWT | 249 |
| | SIYIH | | | | | |
| TM-80 | RSSQSLLHVN | 195 | RMSNLAS | 210 | MQHLQYPFT | 250 |
| | GHTYLY | | | | | |
| TM-81 | KSSQSLLNSK | 196 | FAFFRES | 223 | QQHYSTPFT | 251 |
| | NQKNYLA | | | | | |
| TM-82 | RSSQSLANTY | 197 | GISNRFS | 224 | LQGTHPPRT | 252 |
| | GNTYLS | | | | | |
| TM-83 | RSSQSLVHSN | 198 | KVSNRFS | 209 | SQSTHVPYT | 253 |
| | GITYLH | | | | | |
| TM-84 | RASENIYSSL | 199 | AATNLAD | 225 | QHLWSVPWT | 254 |
| | G | | | | | |
| TM-85 | KASQNVGTNV | 200 | SASYRYS | 226 | QQYNSYPLT | 255 |
| | A | | | | | |
| TM-86 | KSSQSLLNSG | 179 | GASTRES | 211 | QNDHSYPLT | 256 |
| | NQKNYLA | | | | | |
| TM-87 | RASRSISKYL | 201 | SGSTLQS | 227 | QQHNEYPLT | 257 |
| | A | | | | | |
| TM-88 | ITSSDIDDHM | 202 | EGNALRP | 228 | LQSDNLPLT | 258 |
| | N | | | | | |
| TM-89 | RASQDIGSSL | 203 | ATSSLDS | 229 | LQYASSPRT | 259 |
| | N | | | | | |
| TM-90 | RSSQSFVHGN | 204 | KVSHRFS | 230 | FQNSHVPHT | 260 |
| | GNTYLE | | | | | |
| TM-91 | KASQNVYTNV | 205 | SASYRYS | 226 | QQYNSYPLT | 255 |
| | A | | | | | |
| TM-92 | RASKSISKYL | 206 | SGSTLQS | 227 | QQHNEYPWT | 261 |
| | A | | | | | |
| TM-93 | KASQDVGSAV | 207 | WSSTRLP | 231 | QQYTSYPLT | 262 |
| | A | | | | | |
| TM-94 | RSSQSLVYSN | 208 | KVSNRFS | 209 | SQSTHVYT | 263 |
| | GNTYLH | | | | | |

TABLE 4

| | | SEQ ID | | SEQ ID |
|---|---|---|---|---|
| Antibody | Variable Heavy Chain | NO: | Variable Light Chain | NO: |
| TM-54 | QVQMQQSGPVLVKPGASVTL | 4 | DVLMTQTPLSLPVSLGDQASIS | 41 |
| | SCKASGYTFTTYDLNWVKQR | | CRSSQSIVHGNGNTYLEWYLQK | |
| | PGQGLEWIGWIYPRGGSTEY | | PGQSPKLLIYKVSNRFSGVPDR | |
| | NEKFKGKATLTVDTSSSTAY | | FSGSGSGTDFTLKISRVEAEDL | |
| | MELHSLTSEDSAVYFCAKYS | | GVYYCFQGSHVPWTFGGGTKLE | |
| | IYAMDYWGQGTSVTVSS | | MK | |
| TM-56 | QVHLQQSGAELAKPGASVKL | 5 | DIEMTQAAPSVPVTPGESVSIS | 42 |
| | SCKASGYTFTSYWMHWVKQR | | CRSSKSLLQNNGNTYLYWFLQR | |
| | PGQGLEWIGYINPSSGYSKY | | PGQPPQLLIYRMSNLASGVPDR | |
| | NQRFKDKATLTADKSSTTAY | | FSGSGSGTAFTLRISRVEAEDV | |
| | MHLSSLTYADSAVYYCTREG | | GVYYCMQHLEYPYTFGGGTKLE | |
| | GSISDWYFDVWGTGTTVTVS | | IK | |
| | S | | | |
| TM-59 | QVQLQQSGAELAKPGASVKL | 6 | DIVMTQAAPSVPVTPGESVSIS | 43 |
| | SCKASGYTFISYWMHWVKQR | | CRSSKSLLHSNGNTYSYWFLQR | |
| | PGQGLEWIGYVNPSSGYTKN | | PGQSPQLLIYRMSNLASGVPDR | |
| | NQKFKDKATLTADKSSSTAY | | FSGSGSGTAFTLRISRVEAEDV | |
| | MQLSSLTYEDSAVYYCAREG | | GVYYCMQHLEYPYTFGGGTKLE | |
| | GSISDWYFDVWGTGTTVTVS | | IK | |
| | S | | | |
| TM-60 | QVQLQQSGPELVKPGASVKL | 7 | DVLMTQTPLSLPVSLGDQASIS | 44 |
| | SCKASGYTFTSYDINWVKQR | | CRSSQSIVHGNGNTYLEWYLQK | |
| | PGQGLEWIGWIYPRDGNTQY | | PGQSPKLLIYKVSNRFSGVPDR | |
| | IEKLKGKATLTVDTSSSTAY | | FSGSGSGTDFTLKISRVEAEDL | |
| | MELHSLTSEDSAVYFCARWI | | GVYYCFQGSHLPYTFGGGTKLE | |
| | FYAMDYWGQGTSVTVSS | | IK | |
| TM-61 | QVQLQQSGAELAKPGASVKL | 8 | DIVMTQSPSSLSASAGEKVTMS | 45 |
| | SCKASGYTFTNYWMNWVKQR | | CKSSQSLLNSGNQKNYLAWYQQ | |
| | PGQGLEWIGYINPSSDYTKY | | KPGQSPKLLIYGASTRESGVPD | |
| | NQNFKDKATLTADKSSSTAY | | RFTGSGSGTDFTLTISSVQAED | |
| | MQLNSLTYEDSAVYYCTRWG | | LAVYYCQNDSNYPFTFGGGTKL | |
| | GSRSYWYFDVWGAGTTVTVS | | EMK | |
| | S | | | |
| TM-62 | QVQLQQPGTELVRPGSSVKL | 9 | QIVLTQSPAIMSASPGEKVTIS | 46 |
| | SCKAAGYTFTRYWMHWVKQR | | CSASSSVSYMYWYQQKAGSSPK | |
| | PIQGLEWIGNIDPSDSETQY | | PWIHRTSNLASGVPARFSGSGS | |
| | NPKFKDKATLTVDRSSSTAY | | GTSYSLTIRSMEAEDAATYYCQ | |
| | MHLSSLTSEDSAVYYCARLT | | QYHSYPPTFGVGTKLELK | |
| | TLDYAMDYWGQGTSVTVSS | | | |
| TM-63 | QVQLQQPGAELVKFGASVKL | 10 | DIVMTQASPSVSVTPGESVSIS | 47 |
| | SCKASGYTFTSFWMHWVKQR | | CRSSQSLLHVNGNTYLYWFLQR | |
| | PGQGLEWIGNIDPSDSETHY | | PGRSPRLLIYRMSNLASGVPDR | |
| | SQKFKDKATLTVDRSSNTAY | | FSGSGSGTAFTLRISRVEAEDV | |
| | IQLSSLTSEDSAVYYCARSS | | GVYYCMQHLQYPFSFGSGTKLE | |
| | NWDNFDYWGQGTTVTVSS | | MK | |
| TM-64 | QVQLQQPGAELVKPGASVKL | 11 | DIVMTQAAPSVPVTPGESVSIS | 48 |
| | SCKASGYTFTSYWMHWVKQR | | CRSSKSLLHTNGNTYLFWFLQR | |
| | PGHGLEWIGNIDPSDSDTHY | | PGRSPQLLIYRMSNLASGVPDR | |
| | NQNFRGKATLTVDKFSTTAY | | FSGSGSGTAFTLRISRVEAEDV | |
| | MHLSSLTSEDSAVYYCARSR | | GVYYCMQHLEYPFTFGSGTKLE | |
| | GGTYDYGEAMDYWGQGTSVT | | IK | |
| | VSS | | | |
| TM-65 | QVQLQQPGAEFVKPGASVRL | 12 | DIQMNQSPSSLSASLGDTITIT | 49 |
| | SCKTSGYTFTSSWMHWVKQR | | CRASQNINIWLSWYQQKPGNVP | |
| | PGQGLEWIGEINPKNGGTNY | | KLLIYKASNLHTGVPSRFSGSG | |
| | NEKFKTKATLTVDKSSNTAY | | SGTGFTLTISSLQPEDIATYYC | |
| | MQLSSLTSEDSAVYYCTIPT | | LQGQSYPYTFGGGTKLGIK | |
| | WFTYWGQGTLVTVSS | | | |
| TM-66 | EVMLVESGGALVEPGGSLKL | 13 | DVVVTQTPPSLPVSFGDQVSIS | 50 |
| | SCAASGITFSNYAMSWIRQT | | CRSSQSLVNSYGKTFLSWYLHK | |
| | PEKRLEWVATIGSVGSYTYY | | PGQSPQLLIYGISNRFSGVPDR | |
| | PDSVKGRFTISRDNARNTLY | | FSGSGSGTVFTLKISTIKPEDL | |
| | LQMSSLRSEDTAMYYCARQE | | GMYYCLQGTHPPLTFGAGTKLE | |
| | GSSHFDYWGQGTTLTVSS | | MK | |
| TM-68 | EVQLQQSGAELVRPGASVKL | 14 | DIVMTQAAFSNPVTLGTSVSIS | 51 |
| | SCIASGFNIKDDYMHWVRQR | | CSSSKSLLHSSGITYLYWYLQR | |
| | PEQGLEWIGWIDPENGDTEY | | PGQSPQLLIYRMSNLASGVPDR | |
| | ASKFQGKATMTADTSSNTAY | | FSGSGSGTDFTLRISRVEAEDV | |
| | LHLSSLTSEDTAVYYCTKPQ | | GVYYCAQMLERPWTFGGGTKLE | |
| | TATTCWGQGTLVTVSS | | IK | |
| TM-69 | QVQLKESGPDLVQPSQTLSL | 15 | DVVMTQTPLSLPVSLGDQASIS | 52 |
| | TCTVSGFSFTSYGVHWVRQP | | CRSSQSLVHSNGNTYLHWYLQK | |
| | PGKGLEWVGIMGWDDKKYYN | | PGQSPKLLIYKVSNRFSGVPDR | |
| | SALKSRLSISRDTSKNQVFL | | FSGSGSGTDFTLKISRVEAEDL | |
| | KLSSLQTEDTAMYYCTREAG | | GVYFCSQNTHVPLTFGAGTKLE | |
| | PYGMDYWGQGTSVTVSS | | LK | |
| TM-70 | EVQLVESGGALVKPGGSLKL | 16 | DIVLTQSPASLAVSLGQRATIS | 53 |
| | SCAASGFTFSNYAMSWVRQT | | CRASESVDNYGISFMNWFQQKP | |
| | PEKRLEWVATIGYGGSYSYY | | GQPPKLLIYAASNQGSGVPARF | |
| | PDSVKGRFTISRDNAKYTLY | | SGSGSGTDFSLNIHPMEEDDTA | |
| | LQMSGLRSEDTALYYCARQD | | MYFCQQSKDVPWTFGGGTKLAI | |
| | SNYEGVWFAYWGQGTLVT | | K | |
| TM-71 | EVQLVESGGALVKPGGSLKL | 17 | DIVMTQAAPSVPVTPGESVSIS | 54 |
| | SCAASGFTFSNYAMSWVRQT | | CRSSKSLLHTNGNTYLFWFIQR | |
| | PEKRLEWVATIGYGGSYSYY | | PGQSPHLLIYRKSNLASGVPDR | |
| | PDSVKGRFTISRDNAKNTLY | | FSGSGSGTAFTLRISRVEAEDV | |
| | LQMSSLRSEDTALYYCARQD | | GVYYCMQHLEYPFTFGSGTKLE | |
| | SNYEGVWFAYWGQGTLVTVS | | IK | |
| | S | | | |
| TM-72 | QVQLQQPGAELVKPGTSVKL | 18 | DIVMTQAAPSVPVTPGESVSIS | 54 |
| | SCKASGSTFTNYWMHWVMQR | | CRSSKSLLHTNGNTYLFWFIQR | |
| | PGQGLEWIGNIDPSDSDTHY | | PGQSPHLLIYRKSNLASGVPDR | |
| | NQKFKDRAKLTVDKSSSTAY | | FSGSGSGTAFTLRISRVEAEDV | |
| | MQLSGLTSEDSAVYYCARSR | | GVYYCMQHLEYPFTFGSGTKLE | |
| | GGYYDYGEAMDYWGQGTSVT | | IK | |
| | VSS | | | |
| TM-73 | QVQLQQPGAELVKPGASVKL | 19 | QIVLTQSPAIMSASPGEKVTLT | 55 |
| | SCKASGYTFTSHWMHWVKHR | | CSASSGISYIYWYQQRPGSSPR | |
| | PGQGLEWIGEIDPSDSYTYY | | LLIYDTSNLASGVPVRFSGSGS | |
| | NQKFKGKATLTVDKSSSTVY | | ETSYSLTISRLEAEDAATYYCH | |
| | MQLSSLTSEDSAVYYCARNY | | QWRSYPPTFGGGTKLEIR | |
| | HGSGPPFAHWGQGTLVTVSS | | | |
| TM-74 | EVQLQQSGPELVKPGASVKM | 20 | DVVMTQSQKFMSTSVEDRVSIT | 56 |
| | SCKASGYTFTSYVIHWVKQK | | CKASQNVRSAVAWYQQKPGQSP | |
| | PGQGLEWIGFINPNNDGTKY | | KALIYLASNRHTGVPDRFAGSG | |
| | NEKFRGKATLTSDKSSSTAY | | SGTDFTLTISSVQSEDLADYFC | |
| | MELSSLTSEDSAVYYCARPY | | LQHWKYPFTFGSGTKLEIK | |
| | YDYEGYTMDYGGQGTSVTVS | | | |
| | S | | | |
| TM-75 | EVQLQQSGPELVKPGASVKM | 21 | DIVLTQSPASLAVSLGQRATIF | 57 |
| | SCKASGYTFTSYVMHWVKQK | | CRASQSVDYNGISYMHWFQQKP | |
| | PGQGLEWIGYINPYNDGTKY | | GQPPKLLIYAASNLESGIPARF | |
| | NEKFKGKATLTSDKSSSTAY | | SGSGSGTDFTLNIHPVEEEDAA | |
| | MELSSLTSEDSAVYYCARGG | | TYYCQQSIEDPWTFGGGTKLEI | |
| | WFPYAMDYWGQGTTVTVSS | | K | |
| TM-76 | QVTLKESGPGILQPSQTLSL | 22 | QIILTQSPAIMSASLGEEITLT | 58 |
| | TCSFSGFSLSTSGMGVSWIR | | CSASLSISYVHWYQQKSGASPK | |
| | KPSGKGLEWLAHIFWDDDKR | | LLIYGTSNLASGVPSRFSGSGS | |
| | YNLFLKSRLTVSKDTSSNQV | | GTFYSLTISSVEAEDAADYYCH | |
| | FLMITSVDTTDTATYYCARM | | QWSIYRTFGGGTKLEMK | |
| | GRQRDYFDYWGQGTSLTVSS | | | |
| TM-77 | QIQLQQSGAELVRPGASVKL | 23 | DVVMTQIPLTLSVTLGQPASIS | 59 |
| | SCKASGYTFTDYEMHWVKQT | | CKSSQSLFHSNGKTYLNWLLQR | |
| | PVHGLEWIGVIDPETGGTAY | | PGQSPKLLIYMVSKLESGVPDR | |
| | NQMFKGKATLTADKSSSTAY | | FSGSGSGTDFTLKISRVEAEDL | |
| | MDLRSLTSEDSAVYYCVKYG | | GVYYCLQVTHFPLTFGAGTKLE | |
| | IEGYWGQGTTLTVSS | | LK | |
| TM-78 | QVQLQQPGAELVKPGASVKL | 24 | DIVMTQATPSIPVTPGESVSIS | 60 |
| | SCKASGYTFTNHWMHWVRQR | | CRSSKSLLHVNGNTYLFWFLQR | |
| | PGQGLEWIGNIDPSDSDTHY | | PGQSPQLLIYRMSNLASGVPDR | |
| | NHKFKDKATLIVDKSSSTAY | | FSGSGSGTAFTLRISRVEAEDV | |
| | LQLSSLTSEDSAVFYCARSR | | GVYYCMQHLEYPFTFGSGTKLE | |
| | GGYYDYGEAMDYWGQGTSVT | | IK | |
| | VSS | | | |
| TM-79 | EVQLQQSGPELVKPGASVKM | 25 | DIVLTQSPASLAVSLGQRATIS | 61 |
| | SCKASGYTFTNYVMHWLKQK | | CRASKSVSTSGSIYIHWYQQKP | |
| | PGQGLEWIAHITPHNDGTKY | | GQPPKLLIYLTSNLESGVPARF | |
| | NEKFKGKATLTSDRSSSTAY | | SGSGSGTDFTLNIHPVEEEDAA | |
| | MELNSLTSEDSAIYYCARSG | | TYYCQHSGELPWTFGGGARLEI | |
| | QFGRGDYFHYWGQGTTVTVS | | K | |
| | S | | | |
| TM-80 | QVQLQQPGAELVKPGASVKL | 26 | DIVMTQAAPSVPVTPGESVSIS | 62 |
| | SCKASGYTFTTFWMHWVKQR | | CRSSQSLLHVNGHTYLYWFLQR | |
| | PGQGLEWIGNIDPSDSETHY | | PGQSPQLLIYRMSNLASGVPDR | |
| | NQEFKDKATLTVDRSSNTAY | | FSGSGSGTVFTLRISRVETEDV | |
| | MQLSSLTSEDSAVYYCARSS | | GIYYCMQHLQYPFTFGSGTKLE | |
| | NWDNFDYWGQGTAVTVSS | | IK | |
| TM-81 | QVQLQQSGAELVKPGASVKL | 27 | DIVMTQSPSSLAMSVGQKVTMS | 63 |
| | SCKASGYTFTSYWMHWVKQR | | CKSSQSLLNSKNQKNYLAWYQQ | |
| | PGQGLEWIGNIDPSDSEIHY | | KPGQSPKFLVYFAFFRESGVPD | |
| | NQKFKDKATLTVDKSSSTAY | | RFIGSGSGTDFTLTISSVQAED | |
| | IQLSSLTSEDSAVYYCARSP | | LADYFCQQHYSTPFTFGSGTKL | |
| | AGQILDYWGQGTTVTVSS | | EIK | |
| TM-82 | QVQLQQSGAELVRPGTSVRV | 28 | DVVVTQTPLSLPVSFGDQVSIS | 64 |
| | SCKASGYAFTNYLIEWIKQR | | CRSSQSLANTYGNTYLSWYLHT | |
| | PGQGLEWIGVINPGSGITDY | | PGQSPQLLIFGISNRFSGVPDR | |
| | SEKFKGKARLTADKSSSTAY | | FSGSGSGTDFTLKISTIKPEDL | |
| | MQLSSLTSDDSAVYFCTRSP | | GVYYCLQGTHPPRTFGGGTKLE | |
| | FITTVVADYWGQGTTVTVSS | | MK | |
| TM-83 | QVQLQQPGTELVKPGALVKL | 29 | DVVMTQTPLSLPVSLGDQASIS | 65 |
| | SCKASGYTFTTYWIHWVKQR | | CRSSQSLVHSNGITYLHWYLQK | |
| | PGQGLEWIGNINPSNGNTKN | | PGQSPKLLIYKVSNRFSGVPDR | |
| | NERFKTKATLTVDKSSSTAY | | FSGSGSGTDFTLKISRVEAEDL | |
| | MQLSSLTSEDSAVYYCVRGD | | GVYFCSQSTHVPYTFGGGTKLE | |
| | YYGTSYPFWGQGTLVTVSS | | IK | |
| TM-84 | QVQLQQSGAELVKPGASVKM | 30 | DIQMTQSPASLSVSVGETVTIT | 66 |
| | SCKASGYTFTTFPIEWMKQN | | CRASENIYSSLGWYHQKQGKSP | |
| | HGKSLEWIGNFHPYNDDTKY | | QLLVFAATNLADGVPSRFSGSG | |
| | NEKFKGKATLTVEKSSSTVY | | SGTQYSLKINSLQSEDFGTYYC | |
| | LELSRLTSDDSAVYYCARYF | | QHLWSVPWTFGGGTKLEIK | |
| | YGGMDYWGQGTSVTVSS | | | |
| TM-85 | QVQLQQPGAELVRAGTSVKL | 31 | DIAMSQSQKFMSTSVGDRVSVT | 67 |
| | SCKASGYTFIGYWMHWVKQR | | CKASQNVGTNVAWYQQKPGQSP | |
| | PGRGLEWIGRIDPYSGDTRY | | KALIYSASYRYSGVPDRFTGSG | |
| | NEKFKNKATLTVDKPSSTAY | | SGTDFTLTISNVQSEDLAEYFC | |
| | MQLSSLTSEDSAVYYCARYG | | QQYNSYPLTFGAGTKLEMK | |
| | LGHAMDYWGQGTSVTVSS | | | |
| TM-86 | EVQLQQSGPVLVKPGASVKM | 32 | DIVMTQSPSSLSVSAGEKVTMS | 68 |
| | SCKASGYTFTDYYIHWVKQS | | CKSSQSLLNSGNQKNYLAWHQQ | |
| | HGRSLEWIGVISPYNDNTNY | | RPGQPPKLLIYGASTRESGVPD | |
| | NQKFKGKATLTVDKSSSTAY | | RFTGSGSGTEFTLTISSVQAED | |
| | MELNSLTSEDSAVYYCVRTY | | LAVYYCQNDHSYPLTFGAGTKL | |
| | YSNYFDYWGQGTTLTVSS | | ELK | |
| TM-87 | EVQLQQSGPVLVKPGASVKL | 33 | DVQITQSPSYLAASPGETITIN | 69 |
| | SCKASGYTFTDYYMNWVKQS | | CRASRSISKYLAWYQEKPGKTN | |
| | HGKSLEWIGVINPYNGATSY | | KLLIYSGSTLQSGIPSRFSGSG | |
| | NQKFQDKATLTVDKSSSTAY | | SGTDFTLTISSLEPEDFAVYYC | |
| | MGLNSLTSEDSAVYYCANGN | | QQHNEYPLTFGAGTKLELK | |
| | YDWYFDVWGTGTTVTVSS | | | |
| TM-88 | EAQLQQSGPVLVKPGASVKM | 34 | ETTVTQSPASLSMAIGEKVTIR | 70 |
| | SCKTSGYTFTDYYINWVKQS | | CITSSDIDDHMNWYRQKPGEPP | |
| | HGKSLEWIGVINPYSGATYY | | EFLISEGNALRPGVPSRFSSSG | |
| | TQKFKGKATLTVDKSSSTAY | | YGTDFIFTIENILSEDVADYYC | |
| | MELNSQTSEDSAVYYCARGG | | LQSDNLPLTFGAGTKLELK | |
| | YDYGHWYFDVWGTGTTVTVS | | | |
| | S | | | |
| TM-89 | QVQLQQSGAELVRPGTSVKV | 35 | DIQMTQSPSSLSASLGERVSLT | 71 |
| | SCKASGYAFTNYLIEWVKQR | | CRASQDIGSSLNWLQQEPDGTI | |
| | PGQGLEWIGVINPGSGGTDY | | KRLIYATSSLDSGVPKRFSGSR | |
| | NEKFKGKATLTADKSSSTAY | | SGSDYSLTISSLESEDFVDYYC | |
| | MQLSSLTSEDSAVYFCARDP | | LQYASSPRTFGGGTKLEIK | |
| | YSKYVHYPMDYWGQGTSVTV | | | |
| | SS | | | |
| TM-90 | QVHLQQSGPELVKPGASVKL | 36 | DVLMTQTPLSLPVSLGDQASIS | 72 |
| | SCKASGYPFTSYDINWVKQR | | CRSSQSFVHGNGNTYLEWYLQK | |
| | PGQGLEWIGWIYPRDGNAKY | | PGQSPKLLIYKVSHRFSGVPDR | |
| | NEKFKGKATLTVDTSSSTAY | | FSGSGSGTDFTLKITRVEAEDL | |
| | MELHSLTSENSAVYFCARVS | | GVYYCFQNSHVPHTFGGGTKLE | |
| | DYHGGYGMDYWGQGTSVTVS | | IK | |
| | S | | | |
| TM-91 | DVQLQESGPGLVKPSQSLSL | 37 | DIVMTQSQKFMSTSVGDRVSVT | 73 |
| | TCSVTGYSITSGYYWDWIRQ | | CKASQNVYTNVAWYQQKPGQSP | |
| | FPGNKLEWMGYISYDGNNNY | | KPLIYSASYRYSGVPDRFTGSG | |
| | NPSLKNRISITRDTSKNQFF | | SGTDFTLTISNVQSEDLAEYFC | |
| | LKLNSVTTEDTATYYCARAV | | QQYNSYPLTFGGGTKLEIK | |
| | YSVGFAYWGQGTLVTVSS | | | |
| TM-92 | EVQLQQSGPELVKPGASVKI | 38 | DVQITQSPSYLAASPGETITIN | 74 |
| | PCKASGYTFTDYTMDWVKQS | | CRASKSISKYLAWYQEKPGKTN | |
| | PGKSLEWIGHINPNDGGTFY | | KLLIYSGSTLQSGIPSRFSGSG | |
| | NQKFKGKATLTVDKSSSTAY | | SGTDFTLTISSLEPEDFAMYYC | |
| | MELRSLTSEDSAVYYCARWV | | QQHNEYPWTFGGGTKLEIK | |
| | YYYGISYEGGAMDYWGQGTS | | | |
| | VTVSS | | | |
| TM-93 | QVQLKESGPGLVAPSQSLSI | 39 | DIVMTQSHKLMSTSVGDRVSIT | 75 |
| | TCTVSGFSLSSYGISWVRQP | | CKASQDVGSAVAWYQQKPGQSP | |
| | PGKGLEWLGVIWTGGGTNYN | | QLLIYWSSTRLPGVPDRFTGSG | |
| | SALESRLSITKDNSKSQVFL | | SGTDFTLTISNVQSEVLADYFC | |
| | KMNSLQTDDTARYYCARNPP | | QQYTSYPLTFGGGTKLEIK | |
| | KIFYDYLMYFDYWGQGTTVT | | | |
| | VSS | | | |
| TM-94 | EVQLQQSGPELVKPGASVKI | 40 | DVVMTQTPLSLPVSLGDQASIS | 76 |
| | PCKASGYTFTDYNMDWVKQS | | CRSSQSLVYSNGNTYLHWYLQR | |
| | HGKSLEWIGDINPYNGGTLY | | PGQSPKLLIYKVSNRFSGVPDR | |
| | NQKFKGKATLTVDKSSNTAY | | FSGSGSGTDFTLKISRVEAEDL | |
| | MEIRSLTYEDTAVYYCARVG | | GVYFCSQSTHVYTFGGGTKLEI | |
| | YHGTPDYWGQGTTVTVSS | | K | |

### Example 9: Epitope Binning of Anti-TMEM106B Antibodies

Epitope binning of the anti-TMEM106B antibodies was performed by Lake Pharma (Salt Lake City, Nevada, USA) using a pre-mix epitope binning approach. Monoclonal anti-TMEM106B antibodies were immobilized to a CMD 50M chip (Xantec #SPMXCMD50M lot #SCCMD50M0416.a exp 31.03.18. The running buffer was HBS-EP+ with 1 mg/ml BSA. The GST-TMEM106B (truncated) antigen was prepared at a final concentration of 55 nM (corresponding to 2 μg/ml) and mixed with the competing analyte anti-TMEM106B antibodies at a final concentration of 333 nM (corresponding to 50 μg/ml) or compared to a buffer control. Samples were injected for 5 minutes over the array and regenerated after every cycle with 1 minute of two parts Pierce IgG-Elution buffer (ThermoFisher Cat #21004) and 1 part of 10 mM Glycine, pH 2.0 (Carterra).

These results of these experiments are shown below in Table 5.

TABLE 5

| Community 1 | Community 2 | Community 3 | Community 4 |
|---|---|---|---|
| TM-79, TM-82 | TM-54, TM-59, | TM-83 | TM-63, TM-64, |
| | TM-60, TM-61, | | TM-72, TM-78, |
| | TM-76, TM-87, | | TM-80, TM-86, |
| | TM-90, TM-91, | | TM-88 |
| | TM-92, TM-93 | | |

These results showed that anti-TMEM106B antibodies of the present disclosure bin to at least 4 different communities (e.g., anti-TMEM106B antibodies that bin to a particular community bind to the same or overlapping epitope), based on the assay used as described above

### Example 10: Kinetic Characterization of Anti-TMEM106B Antibodies

Binding kinetic characterization of the purified antibodies is performed by various methods, such as by Carterra using a proprietary array SPR instrument (MX-96) as follows. Briefly, antibodies are prepared by diluting to 5 μg/ml in 10 mM Acetate, pH 4.5 (Carterra), at 150 μL/well and then made an additional dilution at 1:10 from there by titrating 11 μL into 100 μL. The antibodies are printed onto a CMD 50M chip (Xantec #SPMXCMD50M lot #SCCMD50M0416.a exp 31.03.18) using the CFM. The chip is activated with 18 mM EDC (Sigma Bioxtra) and 4.5 mM S-NHS (Thermo Fisher) diluted in 100 mM MES, pH 5.5, for 7 minutes, and then antibodies are coupled for 10 minutes at 45 μL/minute. After coupling, the chip is returned to the MX-96 and quenched for 7 minutes using 1 M Ethanolamine pH 8.5 (Carterra).

Printed antibodies are profiled for their ability to bind GST-TMEM106B (truncated) protein (described above). Briefly, GST-TMEM106B (truncated) antigen is diluted to 18 μg/ml (500 nM of 36 kDa fusion protein) by mixing 2.7 μL of 2.0 mg/ml antigen into 298 μL Running buffer (HBS-EP+, Teknova with 1 mg/ml BSA, Sigma), and titrated 50 μl into 200 μl for 5-fold serial dilutions. In addition, binding to GST (Pierce) is assayed under the same conditions to determine the extent of any non-specific binding of the anti-TMEM106B antibodies to GST portion of the TMEM106B-GST fusion protein. Duplicate measurements for each anti-TMEM106B antibody are taken to ensure reproducibility.

Kₒₙ, K_{off} and K_{D} are calculated for each of the anti-TMEM106B antibodies displaying binding to the truncated TMEM106B protein.

### Example 11: Epitope Mapping of Anti-TMEM106B Antibodies

Epitope mapping of the anti-TMEM106B antibodies is performed by any of a number of assays, such as by Pepscan (Lelystad, Netherlands). Using their proprietary CLIPS technology, Pepscan creates a library (>2500) of linear peptides and looped and discontinuous epitope mimics of the human TMEM106B protein. These peptides are made in situ on a proprietary hydrogel of a Pepscan mini-array and binding of anti-TMEM106B antibodies to each peptide is measured using an ELISA-based method.

The linear and CLIPS peptides are synthesized based on the amino acid sequence of the target protein using standard Fmoc-chemistry and deprotected using trifluoric acid with scavengers. The constrained peptides are synthesized on chemical scaffolds in order to reconstruct conformational epitopes, using Chemically Linked Peptides on Scaffolds (CLIPS) technology (Timmerman et al. (2007). For example, the single looped peptides are synthesized containing a dicysteine, which is cyclized by treating with alpha, alpha′-dibromoxylene and the size of the loop is varied by introducing cysteine residues at variable spacing. If other cysteines besides the newly introduced cysteines are present, they are replaced by cysteine-acetamydomethyl. The side-chains of the multiple cysteines in the peptides are coupled to CLIPS templates by reacting onto credit-card format polypropylene PEPSCAN cards (455 peptide formats/card) with a 0.5 mM solution of CLIPS template such as 1,3-bis (bromomethyl) benzene in ammonium bicarbonate (20 mM, pH 7.9)/acetonitrile (1:1(v/v)). The cards are gently shaken in the solution for 30 to 60 minutes while completely covered in solution. Finally, the cards are washed extensively with excess of H2O and sonicated in disrupt-buffer containing 1% SDS/0.1% beta-mercaptoethanol in PBS (pH 7.2) at 70° C. for 30 minutes, followed by sonication in H2O for another 45 minutes. The binding of antibody to each peptide is tested in a PEPSCAN-based ELISA. The 455-well credit card format polypropylene cards containing the covalently linked peptides is incubated with primary antibody solution, for example, consisting of 1 μg/ml diluted in blocking solution, for example 4% horse serum, 5% ovalbumin (w/v) in PBS/1% Tween. After washing, the peptides are incubated with a 1/1000 dilution of antibody peroxidase conjugate for one hour at 25° C. After washing, the peroxidase substrate 2,2′-azino-di-3-ethylbenzthiazoline sulfonate (ABTS) and 2 μl of 3% H2O2 are added. After one hour, the color development is measured. The color development is quantified with a charge coupled device (CCD)—camera and an image processing system (as first described in Slootstra et al., 1996).

Six sets of synthesized peptides are prepared as follows. Set 1: linear peptides of 15 amino acid residue lengths are synthesized from the amino acid sequence of human TMEM106B with an off-set of 1 amino acid residue. Set 2: linear peptides of 15 amino acid residue lengths are synthesized with residues on positions 10 and 11 replaced with Ala; when a native Ala residue occurred on either position 10 or 11, it was replaced with Gly. Set 3: linear peptides of 15 amino acid residue lengths are synthesized in which native Cys residues were replaced with Cys-acetamidomethyl (Cys-acm). Set 4: constrained peptides of 17 amino acid residue lengths are synthesized, with positions 2-16 being 15-mer peptides derived from the amino acid sequence of human TMEM106B; Cys residues are inserted in positions 1 and 17 and joined by means of mP2 CLIPS to create a looped structure. Native Cys within the 15-mers are replaced by Cys-acm. Set 5: constrained peptides of 22 amino acid length are constructed, in which positions 2-21 being 20-mer peptides derived from the amino acid sequence of human TMEM106B with an offset of one amino acid residue; residues on positions 11 and 12 are replaced by “PG” motif to induce a β-turn formation. Cys residues are inserted on positions 1 and 22 and are joined by means of mP2 CLIPS to create a β-strand like structure. Native Cys in these peptides are replaced by Cys-acm. Set 6: combinatorial peptides of 33 amino acid length, with positions 2-16 and 18-32 being 15-mer peptides derived from the human TMEM106B sequence. Cys residues are inserted on positions 1, 17, and 33 and were joined by means of T3 CLIPS to create a double loop structure. Native Cys in these peptides are replaced by Cys-acm. The synthesized peptides correspond to both lumenal and cytoplasmic regions of human TMEM106B.

### Example 13: Downregulation of Cellular TMEM106B by Anti-TMEM106B Antibodies in Cell Lines

The ability of anti-TMEM106B antibodies to reduce or down-regulate cell surface and total cellular protein levels of TMEM106B in various cell lines is evaluated as follows. Cell lines useful for such down-regulation experiments are those identified in the literature as expressing TMEM106B and confirmed in experiments described above as showing significant binding to anti-TMEM106B antibodies. Experiments are performed using, for example, the following cell lines: adenocarcinoma HeLa cells (ATCC CTL-2), gliablastoma U251 cells (Sigma Cat #09063001), A549 human lung carcinoma cells (ATCC CCL-185), and mouse Neuroblastoma cell line Neuro2a (ATCC CCL-131). Preferably, experiments are performed using A549 cells, which display high expression of TMEM106B, as shown above. The cell lines are incubated with various concentrations or amounts of anti-TMEM106B antibodies of the present invention for various time periods and then the levels of TMEM106B remaining associated with the cells is measured using either FACS (for measuring changes in levels of cell surface TMEM106B) or western blot (for measuring changes in levels of total cellular TMEM106B).

HeLa cells, U251 cells, and Neuro2a cells are cultured in Eagle's Minimum Essential Media (EMEM)+10% FBS (fetal bovine serum) and A549 cells are cultured in DMEM+10% FBS, each in either T75 or T150 flasks. When the cells reach >80% confluence, they are detached via application of trypsin-EDTA at 37C, the enzyme is quenched with media (including FBS), washed into fresh media, and distributed into 96-well plates (1×10≡cells per well in 10 μL) for FACS assays or 24 well plates (4×10\hat{ }5 cells/well in 1 mL) for western blot readouts. 24 hours after being plated, anti-TMEM106B antibodies are added to the well (using, for example, 0.1-10 μg/ml final antibody concentration) and allowed to incubate overnight with the target cells at 37° C. For FACS assays, detection of the remaining TMEM106B is performed using direct-dyelight-650 conjugated, non-competing antibodies identified above. For Western blot detection, A549 cells are detached via removal of media, washed with PBS, and followed by the addition of trypsin-EDTA (10 minutes at 37° C.). Trypsin-EDTA is then quenched with media (DMEM+10% FBS), and cells removed from the plates into 96-well round-bottomed plates, washed in PBS, and then lysed via addition of 50 μL lysis buffer (RIPA lysis buffer (MermoFischerScientific Cat #89900)+1:100 HALT protease inhibitor cocktail (MermoFischerScientific Cat #87786). Total protein levels in the lysate can be determined by BCA assay (Pierce, Cat #23225), and equivalent levels of protein loaded onto SDS-PAGE gels and then transferred to a nitrocellulose membrane for Western blot analysis (chemilumenescence, using the iBright system from ThermoFischerScientific).

For FACS experiments, downregulation of TMEM106B is associated with reduced binding of the 2nd, non-competing dylight-conjugated anti-TMEM106B antibody. Percent down regulation is calculated from the differential of the MFI of binding to A549 cells with and without the presence of an anti-TMEM106b antibody during overnight incubation. For Western blot experiments, total protein levels are directly assayed based on the level of chemiluminescent signal, and percent down regulation determined by the ratio of signal from cells treated with or without anti-TMEM106B antibodies.

### Example 14: Downregulation of Cellular TMEM106B by Anti-TMEM106B Antibodies in Primary Cell Cultures

The ability of anti-TMEM106B antibodies of the present invention to reduce or down-regulate cell surface/cellular expression in primary cell cultures is evaluated as follows. Mouse primary cortical neurons are harvested from early postnatal nice (day 0-3) and cultured according to standard methods in the field (Maximov et al., 2007, J. Neu. Meth., 161 75-87). Cultured neurons are then incubated with anti-TMEM106B antibodies in various conditions (1-20 μg/ml, 2-48 hours), harvested, and total TMEM106B levels are quantified using either FACS (for measuring changes in levels of cell surface TMEM106B) or western blot (for measuring changes in levels of total cellular TMEM106B).

Primary cortical neurons are isolated as follows. Briefly, cells in the cortex, hippocampus, or striatum of PO mouse pups are dissociated by incubation for 7 minutes at 37° C. in digestion solution containing 6 mg/ml trypsin (Sigma, Cat #T1005-1G), 0.5 mg/ml DNAse (Sigma, Cat #D5025) and 137 nM NaCl, 5 mM KCl, 7 mM Na2HPO4, and 25 mM HEPES-NaOH, pH 7.2. The dissociated cells containing neurons are then washed once with Hank's balanced salt solution (HBSS) containing 20% fetal bovine serum (FBS) followed by two washes in serum-free HBS, and the further dissociated by gentle pipetting in HBS containing 12 mM MgSO4 and 0.5 mg/ml DNAse. The cell suspension is centrifuged for 10 minutes at 160 g and plated on Matrigel (Collaborative Biomedical Products, Cat #871-275-0004) coated circular glass coverslips (in MEM (Invitrogen) supplemented with B27 (Invitrogen, Cat #17504-044), glucose, transferrin, and 5% fetal bovine serum. For cortical cultures, the cell suspension obtained from the cortex of a single brain is used to plate 12 wells in a 24-well plate. For all cultures, the initial cell density (including glia) at plating varies between 1500 and 2500 cells per square millimeter. When the confluency of glia cells in the culture reaches ˜40-50% (usually 2 days after plating), 50% of the conditioned culture medium is replaced with fresh medium containing 4 mM Ara-C(Sigma). The cultures are maintained in medium containing 2 mM Ara-C at 37° C. and 5% CO₂ until experiments (13-18 DIV).

Treatment with anti-TMEM106B antibodies may be carried out for 1-7 days and at concentrations ranging from 0.001-10 μg/ml. Briefly, anti-TMEM106B antibodies are diluted into culture media and added to the cell cultures, followed by a 1-7 day incubation/culture at standard culture conditions. The neural cultures are then disassociated with trypsin-EDTA and prepared for either FACS or Western blot analysis as described above.

The ability of anti-TMEM106B antibodies to down regulate the levels of TMEM106B is determined by showing either lower cell surface TMEM106B levels as determined by FACS analysis using non-blocking TMEM106B antibodies, or lower overall cell TMEM106B levels detected using Western blot analysis.

### Example 15: In Vivo Downregulation of TMEM106B by Anti-TMEM106B Antibodies

The activity of anti-TMEM106B down-regulating antibodies is further examined using two in vivo mouse model systems. Human/mouse cross-reactive anti-TMEM106B antibodies are tested in wildtype mice, while a BAC transgenic line expressing human TMEM106B under its natural enhancers is used to test human-only and human/cyno cross-reactive anti-TMEM106B antibodies. Anti-TMEM106B antibodies are administered to the mice via intraperitoneal injection and changes in total TMEM106B protein levels subsequently evaluated from different tissue types (liver and frontal cortex isolates) by Western Blot and isolated cells (hepatocytes) via FACS.

### Example 16: Characterization of Interactions Between TMEM106B and TMEM106B Binding Partners

TMEM106B has been shown to interact with various proteins, including various proteins associated with late endosomal/lysosomal compartments. Use of anti-TMEM106B antibodies to block or inhibit the interaction of TMEM106B with any of its various binding partners, such as but not limited to progranulin protein, other TMEM106 protein family members, such as TMEM106B and TMEM106C, clathrin heavy chain (CLTC), the μ1 subunit of adipocyte protein 2 (AP2M1), CHMP2B, microtubule-associated protein 6 (MAP6), lysosomal-associated membrane protein 1 (LAMP1), and vacuolar-ATPase subunit accessory protein 1 may be tested. The extent or degree of an anti-TMEM106B antibody blocking the binding of TMEM106B to any of its binding partners is measured by co-immunoprecipitation of TMEM106B protein in the presence or absence of anti-TMEM106B antibodies, followed by Western blot detection of the binding partners. Alternatively, TMEM106B-expressing cell lines are administered anti-TMEM106B antibodies, and then stained for both TMEM106B and binding partner protein levels using a readout such as co-localization or fluorescence resonance energy transfer (FRET).

### Example 17: The Effect of Anti-TMEM106B Antibodies on Pathology Caused by Progranulin Knockout in an FTLD Mouse Model

Progranulin (GRN) knockout mice are the closest animal model available for human GRN-dependent FTLD. This mouse model recapitulates several of the phenotypes of this disorder. The mice show progressive development of lysosomal abnormalities, lipofuscin accumulation, retinal degeneration, frontotemporal dementia-like behavior, and neuropathology. The mice also display enhanced activation of microglia and astrocytes, and ubiquitination and cytoplasmic accumulation of phosphorylated transactivation response element DNA binding protein-43 (TDP-43) in hippocampal and thalamic neurons. By eighteen months of age, the mice demonstrate impaired spatial learning and memory. Treatment of these mice with anti-TMEM106B antibodies would be expected to ameliorate the various phenotypes and aspects of this disorder.

GRN−/− mice are grown to 6-12 months, and anti-TMEM106B antibodies are injected via IV weekly for 14 weeks. At various timepoints, the mice are assayed for behavioral and phenotypic abnormalities known to be caused by granulin knockout, which are measured using assays such as Open Field Test or Elevated Water Maze. Successful treatment is associated with either improved performance, or a slower rate of decline in behavioral assays. In addition, the postmortem brains of these mice are examined for microgliosis, lysosomal protein levels, general lysosomal activity, TDP-43 aggregates, and lipofuscin accumulation in neurons, PGRN homozygous mice are also tested in a similar fashion to characterize the effect of anti-TMEM106B antibodies on their behavior and lysosomal phenotypes, which are less severe than that observed in the knockout mouse.

### Example 18: The Effect of Anti-TMEM106B Antibodies in Animal Models of Aging, Seizures, Spinal Cord Injury, Retinal Dystrophy, Frontotemporal Dementia, and Alzheimer's Disease

The therapeutic utility of anti-TMEM106B antibodies are also tested in animal models of various disorders, such as, for example, animal models of aging, seizures, spinal cord injury, retinal dystrophy, frontotemporal dementia, and Alzheimer disease, as previously described (e.g., Beattie, M S et al., (2002) Neuron 36, 375-386; Volosin, M et al., (2006) J. Neurosci. 26, 7756-7766; Nykjaer, A et al., (2005) Curr. Opin. Neurobiol. 15, 49-57; Jansen, P et al., (2007) Nat. Neurosci. 10, 1449-1457; Volosin, M et al., (2008) J. Neurosci. 28, 9870-9879; Fahnestock, M et al., (2001) Mol. Cell Neurosci. 18, 210-220; Nakamura, K et al., (2007) Cell Death. Differ. 14, 1552-1554; Yune, T et al., (2007) Brain Res. 1183, 32-42; Wei, Y et al., (2007) Neurosci. Lett. 429, 169-174; Provenzano, M J et al., (2008) Laryngoscope 118, 87-93; Nykjaer, A et al., (2004) Nature 427, 843-848; Harrington, A W et al., (2004) Proc. Natl. Acad. Sci. U.S.A. 101, 6226-6230; Teng, H K et al., (2005) J. Neurosci. 25, 5455-5463; Jansen, P et al., (2007) Nat. Neurosci. 10, 1449-1457; Volosin, M et al., (2008) J. Neurosci. 28, 9870-9879; Fan, Y J et al., (2008) Eur. J. Neurosci. 27, 2380-2390; Al-Shawi, R et al., (2008) Eur. J. Neurosci. 27, 2103-2114; and Yano, H et al., (2009) J. Neurosci. 29, 14790-14802).

Using any one of these animal models, an anti-TMEM106B antibody is administered to the animal in various amounts and over various periods of time. At various timepoints thereafter, the animals are assayed for improvements in behavioral and phenotypic abnormalities associated with each specific animal model of human disease. Successful treatment is associated with either improved performance, or a slower rate of decline in behavioral assays.

What is claimed is:

1. An isolated anti-TMEM106B antibody, wherein the anti-TMEM106B antibody comprises at least one, two, three, four, five, or six HVRs of an antibody selected from the group consisting of: TM-54, TM-56, TM-59, TM-60, TM-61, TM-62, TM-63, TM-64, TM-65, TM-66, TM-70, TM-72, TM-73, TM-74, TM-76, TM-77, TM-78, TM-79, TM-80. TM-82, TM-83, TM-84, TM-85, TM-86, TM-87, TM-88, TM-90, TM-91, TM-92, TM-93, and TM-94, optionally wherein the HVRs are the Kabat, AbM, Chothia, or Contact CDRs.

2. An isolated antibody that binds to human TMEM106B, wherein the antibody comprises a heavy chain variable region and a light chain variable region,
wherein the heavy chain variable region comprises:
an HVR-H1 comprising an amino acid sequence selected from the group consisting of SEQ ID NOs:77-106;
an HVR-H2 comprising an amino acid sequence selected from the group consisting of SEQ ID NOs:107-142; and
an HVR-H3 comprising an amino acid sequence selected from the group consisting of SEQ ID NOs:143-175; and
wherein the light chain variable region comprises:
an HVR-L1 comprising an amino acid sequence selected from the group consisting of SEQ ID NOs:176-208;
an HVR-L2 comprising an amino acid sequence selected from the group consisting of SEQ ID NOs:209-231; and
an HVR-L3 comprising an amino acid sequence selected from the group consisting of SEQ ID NOs:232-263.

3. The antibody of claim 2, wherein the HVR-H1, HVR-H2, HVR-H3, HVR-L1, HVR-L2, and HVR-L3 comprise the amino acid sequences of
(a) SEQ ID NOs:77, 107, 143, 176, 209, and 232, respectively;
(b) SEQ ID NOs:78, 108, 144, 177, 210, and 233, respectively;
(c) SEQ ID Nos:78, 109, 144, 178, 210, and 233, respectively;
(d) SEQ ID Nos:79, 110, 145, 176, 209, and 234, respectively;
(e) SEQ ID Nos:80, 111, 146, 179, 211, and 235, respectively;
(f) SEQ ID Nos:81, 112, 147, 180, 212, and 236, respectively;
(g) SEQ ID Nos:82, 113, 148, 181, 210, and 237, respectively;
(h) SEQ ID Nos:78, 114, 149, 182, 210, and 238, respectively;
(i) SEQ ID Nos:83, 115, 150, 183, 213, and 239, respectively;
(j) SEQ ID Nos:84, 116, 151, 184, 214, and 240, respectively,
(k) SEQ ID Nos:85, 117, 152, 185, 210, and 241, respectively;
(l) SEQ ID Nos:86, 118, 153, 186, 209, and 242, respectively;
(m) SEQ ID Nos:84, 119, 154, 187, 215, and 243, respectively;
(n) SEQ ID Nos:84, 119, 154, 182, 216, and 238, respectively;
(o) SEQ ID Nos:87, 120, 155, 182, 216, and 238, respectively;
(p) SEQ ID Nos:88, 121, 156, 188, 217, and 244, respectively;
(q) SEQ ID Nos:89, 122, 157, 189, 218, and 245, respectively;
(r) SEQ ID Nos:90, 123, 158, 190, 219, and 246, respectively;
(s) SEQ ID Nos:91, 124, 159, 191, 220, and 247, respectively;
(t) SEQ ID Nos:92, 125, 160, 192, 221, and 248, respectively;
(u) SEQ ID Nos:93, 126, 155, 193, 210, and 238, respectively;
(v) SEQ ID Nos:94, 127, 161, 194, 222, and 249, respectively;
(w) SEQ ID Nos:95, 128, 148, 195, 210, and 250, respectively;
(x) SEQ ID Nos:78, 129, 162, 196, 223, and 251, respectively;
(y) SEQ ID Nos:96, 130, 163, 197, 224, and 252, respectively;
(z) SEQ ID Nos:97, 131, 164, 198, 209, and 253, respectively;
(aa) SEQ ID Nos:98, 132, 165, 199, 225, and 254, respectively;
(bb) SEQ ID Nos:99, 133, 166, 200, 226, and 255, respectively;
(cc) SEQ ID Nos:100, 134, 167, 179, 211, and 256, respectively;
(dd) SEQ ID Nos:101, 135, 168, 201, 227, and 257, respectively;
(ee) SEQ ID Nos:102, 136, 169, 202, 228, and 258, respectively;
(ff) SEQ ID Nos:96, 137, 170, 203, 229, and 259, respectively;
(gg) SEQ ID Nos:79, 138, 171, 204, 230, and 260, respectively;
(hh) SEQ ID Nos:103, 139, 172, 205, 226, and 255, respectively;
(ii) SEQ ID Nos:104, 140, 173, 206, 227, and 261, respectively;
(jj) SEQ ID Nos:105, 141, 174, 207, 231, and 262, respectively; or
(kk) SEQ ID Nos:106, 142, 175, 208, 209, and 263, respectively.

4. The antibody of claim 2 or 3, wherein the heavy chain variable region comprises an amino acid sequence selected from the group consisting of SEQ ID NOs:4-40, and wherein the light chain variable region comprises an amino acid sequence selected from the group consisting of SEQ ID NOs:41-76.

5. The antibody of claim 4, wherein the heavy chain variable region and the light chain variable region comprise the amino acid sequences of
(a) SEQ ID NOs:4 and 41, respectively;
(b) SEQ ID NOs:5 and 42, respectively;
(c) SEQ ID NOs:6 and 43, respectively;
(d) SEQ ID NOs:7 and 44, respectively;
(e) SEQ ID NOs:8 and 45, respectively;
(f) SEQ ID NOs:9 and 46, respectively;
(g) SEQ ID NOs:10 and 47, respectively;
(h) SEQ ID NOs:11 and 48, respectively;
(i) SEQ ID NOs:12 and 49, respectively;
(j) SEQ ID NOs:13 and 50, respectively;
(k) SEQ ID NOs:14 and 51, respectively;
(l) SEQ ID NOs:15 and 52, respectively;
(m) SEQ ID NOs:16 and 53, respectively;
(n) SEQ ID NOs:17 and 54, respectively;
(o) SEQ ID NOs:18 and 54, respectively;
(p) SEQ ID NOs:19 and 55, respectively;
(q) SEQ ID NOs:20 and 56, respectively;
(r) SEQ ID NOs:21 and 57, respectively;
(s) SEQ ID NOs:22 and 58, respectively;
(t) SEQ ID NOs:23 and 59, respectively;
(u) SEQ ID NOs:24 and 60, respectively;
(v) SEQ ID NOs:25 and 61, respectively;
(w) SEQ ID NOs:26 and 62, respectively;
(x) SEQ ID NOs:27 and 63, respectively;
(y) SEQ ID NOs:28 and 64, respectively;
(z) SEQ ID NOs:29 and 65, respectively;
(aa) SEQ ID NOs:30 and 66, respectively;
(bb) SEQ ID NOs:31 and 67, respectively;
(cc) SEQ ID NOs:32 and 68, respectively;
(dd) SEQ ID NOs:33 and 69, respectively;
(ee) SEQ ID NOs:34 and 70, respectively;
(ff) SEQ ID NOs:35 and 71, respectively;
(gg) SEQ ID NOs:36 and 72, respectively;
(hh) SEQ ID NOs:37 and 73, respectively;
(ii) SEQ ID NOs:38 and 74, respectively;
(jj) SEQ ID NOs:39 and 75, respectively; or
(kk) SEQ ID NOs:40 and 76, respectively.

6. An isolated antibody that binds to human TMEM106B, wherein the antibody competes with one or more of the antibodies of any one of claims 1-5 for binding to human TMEM106B.

7. An isolated antibody that binds to human TMEM106B, wherein the antibody binds essentially the same or overlapping TMEM106B epitope as the antibody of any one of claim 6.

8. The antibody of any one of claims 1-7, wherein the antibody inhibits the interaction of TMEM106B and at least one TMEM106B ligand or binding partner.

9. The antibody of claim 8, wherein the TMEM106B ligand or binding partner is selected from the group consisting of progranulin, TMEM106A, TMTM106C, clathrin heavy chain, μ1 subunit of adipocyte protein 2, CHMP2B, microtubule-associated protein 6, lysosomal-associated membrane protein 1, and vacuolar-ATPase accessory protein 1.

10. The antibody of any one of claims 1-9, wherein the antibody inhibits proteolysis of TMEM106B.

11. The antibody of any one of claims 1-10, wherein the antibody reduces TDP-43 inclusions.

12. The antibody of any one of claims 1-11, wherein the antibody reduces decline in cognitive and behavioral function, and/or improves cognitive and behavioral function.

13. The antibody of any one of claims 1-12, wherein the antibody is a monoclonal antibody.

14. The antibody of any one of claims 1-13, wherein the antibody is of the IgG class, the IgM class, or the IgA class.

15. The antibody of claim 14, wherein the antibody is of the IgG class and has an IgG1, IgG2, or IgG4 isotype.

16. The antibody of any one of claims 1-15, wherein the antibody is an antibody fragment, optionally wherein the fragment is a Fab, Fab′, Fab′-SH, F(ab′)2, Fv or scFv fragment.

17. The antibody of any one of claims 1-15, wherein the antibody is a full-length antibody.

18. The antibody of any one of claims 1-17, wherein the antibody further comprises:
(a) an antigen facilitating transport across the blood-brain-barrier;
(b) an antigen facilitating transport across the blood-brain-barrier selected from the group consisting of transferrin receptor (TR), insulin receptor (HIR), insulin-like growth factor receptor (IGFR), low-density lipoprotein receptor related proteins 1 and 2 (LPR-1 and 2), diphtheria toxin receptor, CRM197, a llama single domain antibody, TMEM30(A), a protein transduction domain, TAT, Syn-B, penetratin, a poly-arginine peptide, an angiopeptide, and ANG1005;
(c) a disease-causing agent selected from the group consisting of disease-causing peptides or proteins or, disease-causing nucleic acids, wherein the disease-causing nucleic acids are antisense GGCCCC (G2C4) repeat-expansion RNA, the disease-causing proteins are selected from the group consisting of amyloid beta, oligomeric amyloid beta, amyloid beta plaques, amyloid precursor protein or fragments thereof, Tau, IAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin 1, ataxin 2, ataxin 3, ataxin 7, ataxin 8, ataxin 10, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides;
(d) ligands and/or proteins expressed on immune cells, wherein the ligands and/or proteins selected from the group consisting of CD40, OX40, ICOS, CD28, CD137/4-1BB, CD27, GITR, PD-L1, CTLA-4, PD-L2, PD-1, B7-H3, B7-H4, HVEM, BTLA, KIR, GAL9, TIM3, A2AR, LAG-3, and phosphatidylserine; and
(e) a protein, lipid, polysaccharide, or glycolipid expressed on one or more tumor cells.

19. An isolated polynucleotide comprising a nucleic acid sequence encoding the antibody of any one of claims 1-18.

20. An isolated polynucleotide comprising a nucleic acid sequence encoding the heavy chain variable region or heavy chain of the antibody or antigen-binding fragment thereof of any one of claims 1-18.

21. An isolated polynucleotide comprising a nucleic acid sequence encoding the light chain variable region or light chain of the antibody or antigen-binding fragment thereof of any one of claims 1-18.

22. A vector comprising the nucleic acid of any one of claims 19-21.

23. An isolated host cell comprising (a) the isolated polynucleotide of any one of claims 19-21, (b) the vector of claim 22, or (c) a first vector comprising the polynucleotide of claim 20 and a second vector comprising the polynucleotide of claim 21.

24. A method of producing an antibody that binds to human TMEM106B, comprising culturing the cell of claim 23 so that the antibody is produced.

25. The method of claim 24, further comprising isolating the antibody produced by the cell.

26. A pharmaceutical composition comprising the antibody of any one of claims 1-18 and a pharmaceutically acceptable carrier.

27. A method of preventing, reducing risk, or treating an individual having a disease, disorder, condition, or injury selected from the group consisting of a neurodegenerative disorder, a disorder characterized by the presence of TDP-43 inclusions, a TDP-43 proteinopathy, frontotemporal lobar degeneration, frontotemporal dementia, frontotemporal lobar degeneration with TDP-43 inclusions, frontotemporal dementia with progranulin mutations, frontotemporal dementia with C90rf72 mutations, hippocampal sclerosis, hippocampal sclerosis of aging, Alzheimer's disease, Lewy body dementia, cognitive impairment, cognitive impairment associated with amyotrophic lateral sclerosis, cognitive impairment in chronic traumatic encephalopathy (CTE), and cancer, the method comprising administering to an individual in need thereof a therapeutically effective amount of the antibody of any one of claims 1-18 or the pharmaceutical composition of claim 26.

28. A method of preventing, reducing risk, or treating an individual having a disease, disorder, condition, or injury selected from the group consisting of a disorder associated with inflammatory cell debris or protein aggregates, abnormal circulating myeloid cells, unhealthy aging, age related brain atrophy, age-associated traits, age-associated inflammation, age-associated neuronal loss, age-associated cognitive deficits, a hypomyelinating disorder, and metastasis, the method comprising administering to an individual in need thereof a therapeutically effective amount of the antibody of any one of claims 1-18 or the pharmaceutical composition of claim 26.

29. A method of preventing, reducing risk, or treating an individual having a disease, disorder, condition, or injury caused by or associated with over expression or increased activity of TMEM106B, the method comprising administering to an individual in need thereof a therapeutically effective amount of the antibody of any one of claims 1-18 or the pharmaceutical composition of claim 26.


SEQUENCE LISTING (267 sequences)

SEQ ID NO: 1 - protein, 274 aa, Artificial Sequence
  Human TMEM106B
MGKSLSHLPLHSSKEDAYDGVTSENMRNGLVNSEVHNEDGRNGDVSQFPYVEFTGRDSVTCPTCQGTGRIPRGQENQLVALIPYSDQRLRPRRTKLYVMASVFVCLLLSGLAVFFLFPRSIDVKYIGVKSAYVSYDVQKRTIYLNITNTLNITNNNYYSVEVENITAQVQFSKTVIGKARLNNITIIGPLDMKQIDYTVPTVIAEEMSYMYDFCTLISIKVHNIVLMMQVTVTTTYFGHSEQISQERYQYVDCGRNTTYQLGQSEYLNVLQPQQ

SEQ ID NO: 2 - protein, 274 aa, Artificial Sequence
  Mouse TMEM106B
GKSLSHLPLHSNKEDGYDGVTSTDNMRNGLVSSEVHNEDGRNGDVSQFPYVEFTGRDSVTCPTCQGTGRIPRGQENQLVALIPYSDQRLRPRRTKLYVMASVFVCLLLSGLAVFFLFPRSIEVKYIGVKSAYVSYDAEKRTIYLNITNTLNITNNNYYSVEVENITAQVQFSKTVIGKARLNNITNIGPLDMKQIDYTVPTVIAEEMSYMYDFCTLLSIKVHNIVLMMQVTVTTAYFGHSEQISQERYQYVDCGRNTTYQLAQSEYLNVLQPQQ

SEQ ID NO: 3 - protein, 274 aa, Artificial Sequence
  Cynomolgus TMEM106B
MGKSLSHLPLHSSKEDAYDGVTSENMRNGLVNSEVHNEDGRNGDVSQFPYVEFTGRDSVTRPTCQGTGRIPRGQENQLVALIPYSDQRLRPRRTKLYVMASVFVCLLLSGLAVFFLFPRSIDVKYIGVKSAYVSYDVQKRTIYLNITNTLNITNNNYYSVEVENITAQVQFAKTVIGKARLNNITHIGPLDMKQIDYTVPTVIAEEMSYMYDFCTLISIKVHNIVLMMQVTVTTTYFGHSEQISQERYQYVDCGRNTTYQLGQSEYLNVLQPQQ

SEQ ID NO: 4 - protein, 117 aa, Artificial Sequence
  TM-54 Antibody Variable Heavy Chain
QVQMQQSGPVLVKPGASVTLSCKASGYTFTTYDLNWVKQRPGQGLEWIGWIYPRGGSTEYNEKFKGKATLTVDTSSSTAYMELHSLTSEDSAVYFCAKYSIYAMDYWGQGTSVTVSS

SEQ ID NO: 5 - protein, 121 aa, Artificial Sequence
  TM-56 Antibody Variable Heavy Chain
QVHLQQSGAELAKPGASVKLSCKASGYTFTSYWMHWVKQRPGQGLEWIGYINPSSGYSKYNQRFKDKATLTADKSSTTAYMHLSSLTYADSAVYYCTREGGSISDWYFDVWGTGTTVTVSS

SEQ ID NO: 6 - protein, 121 aa, Artificial Sequence
  TM-59 Antibody Variable Heavy Chain
QVQLQQSGAELAKPGASVKLSCKASGYTFISYWMHWVKQRPGQGLEWIGYVNPSSGYTKNNQKFKDKATLTADKSSSTAYMQLSSLTYEDSAVYYCAREGGSISDWYFDVWGTGTTVTVSS

SEQ ID NO: 7 - protein, 117 aa, Artificial Sequence
  TM-60 Antibody Variable Heavy Chain
QVQLQQSGPELVKPGASVKLSCKASGYTFTSYDINWVKQRPGQGLEWIGWIYPRDGNTQYIEKLKGKATLTVDTSSSTAYMELHSLTSEDSAVYFCARWIFYAMDYWGQGTSVTVSS

SEQ ID NO: 8 - protein, 121 aa, Artificial Sequence
  TM-61 Antibody Variable Heavy Chain
QVQLQQSGAELAKPGASVKLSCKASGYTFTNYWMNWVKQRPGQGLEWIGYINPSSDYTKYNQNFKDKATLTADKSSSTAYMQLNSLTYEDSAVYYCTRWGGSRSYWYFDVWGAGTTVTVSS

SEQ ID NO: 9 - protein, 119 aa, Artificial Sequence
  TM-62 Antibody Variable Heavy Chain
QVQLQQPGTELVRPGSSVKLSCKAAGYTFTRYWMHWVKQRPIQGLEWIGNIDPSDSETQYNPKFKDKATLTVDRSSSTAYMHLSSLTSEDSAVYYCARLTTLDYAMDYWGQGTSVTVSS

SEQ ID NO: 10 - protein, 118 aa, Artificial Sequence
  TM-63 Antibody Variable Heavy Chain
QVQLQQPGAELVKFGASVKLSCKASGYTFTSFWMHWVKQRPGQGLEWIGNIDPSDSETHYSQKFKDKATLTVDRSSNTAYIQLSSLTSEDSAVYYCARSSNWDNFDYWGQGTTVTVSS

SEQ ID NO: 11 - protein, 123 aa, Artificial Sequence
  TM-64 ANTIBODY VARIABLE HEAVY CHAIN
QVQLQQPGAELVKPGASVKLSCKASGYTFTSYWMHWVKQRPGHGLEWIGNIDPSDSDTHYNQNFRGKATLTVDKFSTTAYMHLSSLTSEDSAVYYCARSRGGTYDYGEAMDYWGQGTSVTVSS

SEQ ID NO: 12 - protein, 115 aa, Artificial Sequence
  TM-65 ANTIBODY VARIABLE HEAVY CHAIN
QVQLQQPGAEFVKPGASVRLSCKTSGYTFTSSWMHWVKQRPGQGLEWIGEINPKNGGTNYNEKFKTKATLTVDKSSNTAYMQLSSLTSEDSAVYYCTIPTWFTYWGQGTLVTVSS

SEQ ID NO: 13 - protein, 118 aa, Artificial Sequence
  TM-66 ANTIBODY VARIABLE HEAVY CHAIN
EVMLVESGGALVEPGGSLKLSCAASGITFSNYAMSWIRQTPEKRLEWVATIGSVGSYTYYPDSVKGRFTISRDNARNTLYLQMSSLRSEDTAMYYCARQEGSSHFDYWGQGTTLTVSS

SEQ ID NO: 14 - protein, 116 aa, Artificial Sequence
  TM-68 ANTIBODY VARIABLE HEAVY CHAIN
EVQLQQSGAELVRPGASVKLSCTASGFNIKDDYMHWVRQRPEQGLEWIGWIDPENGDTEYASKFQGKATMTADTSSNTAYLHLSSLTSEDTAVYYCTKPQTATTCWGQGTLVTVSS

SEQ ID NO: 15 - protein, 117 aa, Artificial Sequence
  TM-69 ANTIBODY VARIABLE HEAVY CHAIN
QVQLKESGPDLVQPSQTLSLTCTVSGFSFTSYGVHWVRQPPGKGLEWVGIMGWDDKKYYNSALKSRLSISRDTSKNQVFLKLSSLQTEDTAMYYCTREAGPYGMDYWGQGTSVTVSS

SEQ ID NO: 16 - protein, 118 aa, Artificial Sequence
  TM-70 ANTIBODY VARIABLE HEAVY CHAIN
EVQLVESGGALVKPGGSLKLSCAASGFTFSNYAMSWVRQTPEKRLEWVATIGYGGSYSYYPDSVKGRFTISRDNAKYTLYLQMSGLRSEDTALYYCARQDSNYEGVWFAYWGQGTLVT

SEQ ID NO: 17 - protein, 121 aa, Artificial Sequence
  TM-71 ANTIBODY VARIABLE HEAVY CHAIN
EVQLVESGGALVKPGGSLKLSCAASGFTFSNYAMSWVRQTPEKRLEWVATIGYGGSYSYYPDSVKGRFTISRDNAKNTLYLQMSSLRSEDTALYYCARQDSNYEGVWFAYWGQGTLVTVSS

SEQ ID NO: 18 - protein, 123 aa, Artificial Sequence
  TM-72 ANTIBODY VARIABLE HEAVY CHAIN
QVQLQQPGAELVKPGTSVKLSCKASGSTFTNYWMHWVMQRPGQGLEWIGNIDPSDSDTHYNQKFKDRAKLTVDKSSSTAYMQLSGLTSEDSAVYYCARSRGGYYDYGEAMDYWGQGTSVTVSS

SEQ ID NO: 19 - protein, 120 aa, Artificial Sequence
  TM-73 ANTIBODY VARIABLE HEAVY CHAIN
QVQLQQPGAELVKPGASVKLSCKASGYTFTSHWMHWVKHRPGQGLEWIGEIDPSDSYTYYNQKFKGKATLTVDKSSSTVYMQLSSLTSEDSAVYYCARNYHGSGPPFAHWGQGTLVTVSS

SEQ ID NO: 20 - protein, 121 aa, Artificial Sequence
  TM-74 ANTIBODY VARIABLE HEAVY CHAIN
EVQLQQSGPELVKPGASVKMSCKASGYTFTSYVIHWVKQKPGQGLEWIGFINPNNDGTKYNEKFRGKATLTSDKSSSTAYMELSSLTSEDSAVYYCARPYYDYEGYTMDYGGQGTSVTVSS

SEQ ID NO: 21 - protein, 119 aa, Artificial Sequence
  TM-75 ANTIBODY VARIABLE HEAVY CHAIN
EVQLQQSGPELVKPGASVKMSCKASGYTFTSYVMHWVKQKPGQGLEWIGYINPYNDGTKYNEKFKGKATLTSDKSSSTAYMELSSLTSEDSAVYYCARGGWFPYAMDYWGQGTTVTVSS

SEQ ID NO: 22 - protein, 120 aa, Artificial Sequence
  TM-76 ANTIBODY VARIABLE HEAVY CHAIN
QVTLKESGPGILQPSQTLSLTCSFSGFSLSTSGMGVSWIRKPSGKGLEWLAHIFWDDDKRYNLFLKSRLTVSKDTSSNQVFLMITSVDTTDTATYYCARMGRQRDYFDYWGQGTSLTVSS

SEQ ID NO: 23 - protein, 115 aa, Artificial Sequence
  TM-77 ANTIBODY VARIABLE HEAVY CHAIN
QIQLQQSGAELVRPGASVKLSCKASGYTFTDYEMHWVKQTPVHGLEWIGVIDPETGGTAYNQMFKGKATLTADKSSSTAYMDLRSLTSEDSAVYYCVKYGIEGYWGQGTTLTVSS

SEQ ID NO: 24 - protein, 123 aa, Artificial Sequence
  TM-78 ANTIBODY VARIABLE HEAVY CHAIN
QVQLQQPGAELVKPGASVKLSCKASGYTFTNHWMHWVRQRPGQGLEWIGNIDPSDSDTHYNHKFKDKATLIVDKSSSTAYLQLSSLTSEDSAVFYCARSRGGYYDYGEAMDYWGQGTSVTVSS

SEQ ID NO: 25 - protein, 121 aa, Artificial Sequence
  TM-79 ANTIBODY VARIABLE HEAVY CHAIN
EVQLQQSGPELVKPGASVKMSCKASGYTFTNYVMHWLKQKPGQGLEWIAHITPHNDGTKYNEKFKGKATLTSDRSSSTAYMELNSLTSEDSAIYYCARSGQFGRGDYFHYWGQGTTVTVSS

SEQ ID NO: 26 - protein, 118 aa, Artificial Sequence
  TM-80 ANTIBODY VARIABLE HEAVY CHAIN
QVQLQQPGAELVKPGASVKLSCKASGYTFTTFWMHWVKQRPGQGLEWIGNIDPSDSETHYNQEFKDKATLTVDRSSNTAYMQLSSLTSEDSAVYYCARSSNWDNFDYWGQGTAVTVSS

SEQ ID NO: 27 - protein, 118 aa, Artificial Sequence
  TM-81 ANTIBODY VARIABLE HEAVY CHAIN
QVQLQQSGAELVKPGASVKLSCKASGYTFTSYWMHWVKQRPGQGLEWIGNIDPSDSEIHYNQKFKDKATLTVDKSSSTAYIQLSSLTSEDSAVYYCARSPAGQILDYWGQGTTVTVSS

SEQ ID NO: 28 - protein, 120 aa, Artificial Sequence
  TM-82 ANTIBODY VARIABLE HEAVY CHAIN
QVQLQQSGAELVRPGTSVRVSCKASGYAFTNYLIEWIKQRPGQGLEWIGVINPGSGITDYSEKFKGKARLTADKSSSTAYMQLSSLTSDDSAVYFCTRSPFITTVVADYWGQGTTVTVSS

SEQ ID NO: 29 - protein, 119 aa, Artificial Sequence
  TM-83 ANTIBODY VARIABLE HEAVY CHAIN
QVQLQQPGTELVKPGALVKLSCKASGYTFTTYWIHWVKQRPGQGLEWIGNINPSNGNTKNNERFKTKATLTVDKSSSTAYMQLSSLTSEDSAVYYCVRGDYYGTSYPFWGQGTLVTVSS

SEQ ID NO: 30 - protein, 117 aa, Artificial Sequence
  TM-84 ANTIBODY VARIABLE HEAVY CHAIN
QVQLQQSGAELVKPGASVKMSCKASGYTFTTFPIEWMKQNHGKSLEWIGNFHPYNDDTKYNEKFKGKATLTVEKSSSTVYLELSRLTSDDSAVYYCARYFYGGMDYWGQGTSVTVSS

SEQ ID NO: 31 - protein, 118 aa, Artificial Sequence
  TM-85 ANTIBODY VARIABLE HEAVY CHAIN
QVQLQQPGAELVRAGTSVKLSCKASGYTFIGYWMHWVKQRPGRGLEWIGRIDPYSGDTRYNEKFKNKATLTVDKPSSTAYMQLSSLTSEDSAVYYCARYGLGHAMDYWGQGTSVTVSS

SEQ ID NO: 32 - protein, 118 aa, Artificial Sequence
  TM-86 ANTIBODY VARIABLE HEAVY CHAIN
EVQLQQSGPVLVKPGASVKMSCKASGYTFTDYYIHWVKQSHGRSLEWIGVISPYNDNTNYNQKFKGKATLTVDKSSSTAYMELNSLTSEDSAVYYCVRTYYSNYFDYWGQGTTLTVSS

SEQ ID NO: 33 - protein, 118 aa, Artificial Sequence
  TM-87 ANTIBODY VARIABLE HEAVY CHAIN
EVQLQQSGPVLVKPGASVKLSCKASGYTFTDYYMNWVKQSHGKSLEWIGVINPYNGATSYNQKFQDKATLTVDKSSSTAYMGLNSLTSEDSAVYYCANGNYDWYFDVWGTGTTVTVSS

SEQ ID NO: 34 - protein, 121 aa, Artificial Sequence
  TM-88 ANTIBODY VARIABLE HEAVY CHAIN
EAQLQQSGPVLVKPGASVKMSCKTSGYTFTDYYINWVKQSHGKSLEWIGVINPYSGATYYTQKFKGKATLTVDKSSSTAYMELNSQTSEDSAVYYCARGGYDYGHWYFDVWGTGTTVTVSS

SEQ ID NO: 35 - protein, 122 aa, Artificial Sequence
  TM-89 ANTIBODY VARIABLE HEAVY CHAIN
QVQLQQSGAELVRPGTSVKVSCKASGYAFTNYLIEWVKQRPGQGLEWIGVINPGSGGTDYNEKFKGKATLTADKSSSTAYMQLSSLTSEDSAVYFCARDPYSKYVHYPMDYWGQGTSVTVSS

SEQ ID NO: 36 - protein, 121 aa, Artificial Sequence
  TM-90 ANTIBODY VARIABLE HEAVY CHAIN
QVHLQQSGPELVKPGASVKLSCKASGYPFTSYDINWVKQRPGQGLEWIGWIYPRDGNAKYNEKFKGKATLTVDTSSSTAYMELHSLTSENSAVYFCARVSDYHGGYGMDYWGQGTSVTVSS

SEQ ID NO: 37 - protein, 118 aa, Artificial Sequence
  TM-91 ANTIBODY VARIABLE HEAVY CHAIN
DVQLQESGPGLVKPSQSLSLTCSVTGYSITSGYYWDWIRQFPGNKLEWMGYISYDGNNNYNPSLKNRISITRDTSKNQFFLKLNSVTTEDTATYYCARAVYSVGFAYWGQGTLVTVSS

SEQ ID NO: 38 - protein, 125 aa, Artificial Sequence
  TM-92 ANTIBODY VARIABLE HEAVY CHAIN
EVQLQQSGPELVKPGASVKIPCKASGYTFTDYTMDWVKQSPGKSLEWIGHINPNDGGTFYNQKFKGKATLTVDKSSSTAYMELRSLTSEDSAVYYCARWVYYYGISYEGGAMDYWGQGTSVTVSS

SEQ ID NO: 39 - protein, 123 aa, Artificial Sequence
  TM-93 ANTIBODY VARIABLE HEAVY CHAIN
QVQLKESGPGLVAPSQSLSITCTVSGFSLSSYGISWVRQPPGKGLEWLGVIWTGGGTNYNSALESRLSITKDNSKSQVFLKMNSLQTDDTARYYCARNPPKIFYDYLMYFDYWGQGTTVTVSS

SEQ ID NO: 40 - protein, 118 aa, Artificial Sequence
  TM-94 ANTIBODY VARIABLE HEAVY CHAIN
EVQLQQSGPELVKPGASVKIPCKASGYTFTDYNMDWVKQSHGKSLEWIGDINPYNGGTLYNQKFKGKATLTVDKSSNTAYMEIRSLTYEDTAVYYCARVGYHGTPDYWGQGTTVTVSS

SEQ ID NO: 41 - protein, 112 aa, Artificial Sequence
  TM-54 ANTIBODY VARIABLE LIGHT CHAIN
DVLMTQTPLSLPVSLGDQASISCRSSQSIVHGNGNTYLEWYLQKPGQSPKLLIYKVSNRFSGVPDRFSGSGSGTDFTLKISRVEAEDLGVYYCFQGSHVPWTFGGGTKLEMK

SEQ ID NO: 42 - protein, 112 aa, Artificial Sequence
  TM-56 ANTIBODY VARIABLE LIGHT CHAIN
DIEMTQAAPSVPVTPGESVSISCRSSKSLLQNNGNTYLYWFLQRPGQPPQLLIYRMSNLASGVPDRFSGSGSGTAFTLRISRVEAEDVGVYYCMQHLEYPYTFGGGTKLEIK

SEQ ID NO: 43 - protein, 112 aa, Artificial Sequence
  TM-59 ANTIBODY VARIABLE LIGHT CHAIN
DIVMTQAAPSVPVTPGESVSISCRSSKSLLHSNGNTYSYWFLQRPGQSPQLLIYRMSNLASGVPDRFSGSGSGTAFTLRISRVEAEDVGVYYCMQHLEYPYTFGGGTKLEIK

SEQ ID NO: 44 - protein, 112 aa, Artificial Sequence
  TM-60 VARIABLE LIGHT CHAIN
DVLMTQTPLSLPVSLGDQASISCRSSQSIVHGNGNTYLEWYLQKPGQSPKLLIYKVSNRFSGVPDRFSGSGSGTDFTLKISRVEAEDLGVYYCFQGSHLPYTFGGGTKLEIK

SEQ ID NO: 45 - protein, 113 aa, Artificial Sequence
  TM-61 ANTIBODY VARIABLE LIGHT CHAIN
DIVMTQSPSSLSASAGEKVTMSCKSSQSLLNSGNQKNYLAWYQQKPGQSPKLLIYGASTRESGVPDRFTGSGSGTDFTLTISSVQAEDLAVYYCQNDSNYPFTFGGGTKLEMK

SEQ ID NO: 46 - protein, 106 aa, Artificial Sequence
  TM-62 ANTIBODY VARIABLE LIGHT CHAIN
QIVLTQSPAIMSASPGEKVTISCSASSSVSYMYWYQQKAGSSPKPWIHRTSNLASGVPARFSGSGSGTSYSLTIRSMEAEDAATYYCQQYHSYPPTFGVGTKLELK

SEQ ID NO: 47 - protein, 112 aa, Artificial Sequence
  TM-63 ANTIBODY VARIABLE LIGHT CHAIN
DIVMTQASPSVSVTPGESVSISCRSSQSLLHVNGNTYLYWFLQRPGRSPRLLIYRMSNLASGVPDRFSGSGSGTAFTLRISRVEAEDVGVYYCMQHLQYPFSFGSGTKLEMK

SEQ ID NO: 48 - protein, 112 aa, Artificial Sequence
  TM-64 ANTIBODY VARIABLE LIGHT CHAIN
DIVMTQAAPSVPVTPGESVSISCRSSKSLLHTNGNTYLFWFLQRPGRSPQLLIYRMSNLASGVPDRFSGSGSGTAFTLRISRVEAEDVGVYYCMQHLEYPFTFGSGTKLEIK

SEQ ID NO: 49 - protein, 107 aa, Artificial Sequence
  TM-65 ANTIBODY VARIABLE LIGHT CHAIN
DIQMNQSPSSLSASLGDTITITCRASQNINIWLSWYQQKPGNVPKLLIYKASNLHTGVPSRFSGSGSGTGFTLTISSLQPEDIATYYCLQGQSYPYTFGGGTKLGIK

SEQ ID NO: 50 - protein, 112 aa, Artificial Sequence
  TM-66 ANTIBODY VARIABLE LIGHT CHAIN
DVVVTQTPPSLPVSFGDQVSISCRSSQSLVNSYGKTFLSWYLHKPGQSPQLLIYGISNRFSGVPDRFSGSGSGTVFTLKISTIKPEDLGMYYCLQGTHPPLTFGAGTKLEMK

SEQ ID NO: 51 - protein, 112 aa, Artificial Sequence
  TM-68 ANTIBODY VARIABLE LIGHT CHAIN
DIVMTQAAFSNPVTLGTSVSISCSSSKSLLHSSGITYLYWYLQRPGQSPQLLIYRMSNLASGVPDRFSGSGSGTDFTLRISRVEAEDVGVYYCAQMLERPWTFGGGTKLEIK

SEQ ID NO: 52 - protein, 112 aa, Artificial Sequence
  TM-69 ANTIBODY VARIABLE LIGHT CHAIN
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPLTFGAGTKLELK

SEQ ID NO: 53 - protein, 111 aa, Artificial Sequence
  TM-70 ANTIBODY VARIABLE LIGHT CHAIN
DIVLTQSPASLAVSLGQRATISCRASESVDNYGISFMNWFQQKPGQPPKLLIYAASNQGSGVPARFSGSGSGTDFSLNIHPMEEDDTAMYFCQQSKDVPWTFGGGTKLAIK

SEQ ID NO: 54 - protein, 112 aa, Artificial Sequence
  TM-71 & TM-72 ANTIBODY VARIABLE LIGHT CHAIN
DIVMTQAAPSVPVTPGESVSISCRSSKSLLHTNGNTYLFWFIQRPGQSPHLLIYRKSNLASGVPDRFSGSGSGTAFTLRISRVEAEDVGVYYCMQHLEYPFTFGSGTKLEIK

SEQ ID NO: 55 - protein, 106 aa, Artificial Sequence
  TM-73 ANTIBODY VARIABLE LIGHT CHAIN
QIVLTQSPAIMSASPGEKVTLTCSASSGISYIYWYQQRPGSSPRLLIYDTSNLASGVPVRFSGSGSETSYSLTISRLEAEDAATYYCHQWRSYPPTFGGGTKLEIR

SEQ ID NO: 56 - protein, 107 aa, Artificial Sequence
  TM-74 ANTIBODY VARIABLE LIGHT CHAIN
DVVMTQSQKFMSTSVEDRVSITCKASQNVRSAVAWYQQKPGQSPKALIYLASNRHTGVPDRFAGSGSGTDFTLTISSVQSEDLADYFCLQHWKYPFTFGSGTKLEIK

SEQ ID NO: 57 - protein, 111 aa, Artificial Sequence
  TM-75 ANTIBODY VARIABLE LIGHT CHAIN
DIVLTQSPASLAVSLGQRATIFCRASQSVDYNGISYMHWFQQKPGQPPKLLIYAASNLESGIPARFSGSGSGTDFTLNIHPVEEEDAATYYCQQSIEDPWTFGGGTKLEIK

SEQ ID NO: 58 - protein, 105 aa, Artificial Sequence
  TM-76 ANTIBODY VARIABLE LIGHT CHAIN
QIILTQSPAIMSASLGEEITLTCSASLSISYVHWYQQKSGASPKLLIYGTSNLASGVPSRFSGSGSGTFYSLTISSVEAEDAADYYCHQWSIYRTFGGGTKLEMK

SEQ ID NO: 59 - protein, 112 aa, Artificial Sequence
  TM-77 ANTIBODY VARIABLE LIGHT CHAIN
DVVMTQIPLTLSVTLGQPASISCKSSQSLFHSNGKTYLNWLLQRPGQSPKLLIYMVSKLESGVPDRFSGSGSGTDFTLKISRVEAEDLGVYYCLQVTHFPLTFGAGTKLELK

SEQ ID NO: 60 - protein, 112 aa, Artificial Sequence
  TM-78 ANTIBODY VARIABLE LIGHT CHAIN
DIVMTQATPSIPVTPGESVSISCRSSKSLLHVNGNTYLFWFLQRPGQSPQLLIYRMSNLASGVPDRFSGSGSGTAFTLRISRVEAEDVGVYYCMQHLEYPFTFGSGTKLEIK

SEQ ID NO: 61 - protein, 111 aa, Artificial Sequence
  TM-79 ANTIBODY VARIABLE LIGHT CHAIN
DIVLTQSPASLAVSLGQRATISCRASKSVSTSGSIYIHWYQQKPGQPPKLLIYLTSNLESGVPARFSGSGSGTDFTLNIHPVEEEDAATYYCQHSGELPWTFGGGARLEIK

SEQ ID NO: 62 - protein, 112 aa, Artificial Sequence
  TM-80 ANTIBODY VARIABLE LIGHT CHAIN
DIVMTQAAPSVPVTPGESVSISCRSSQSLLHVNGHTYLYWFLQRPGQSPQLLIYRMSNLASGVPDRFSGSGSGTVFTLRISRVETEDVGIYYCMQHLQYPFTFGSGTKLEIK

SEQ ID NO: 63 - protein, 113 aa, Artificial Sequence
  TM-81 ANTIBODY VARIABLE LIGHT CHAIN
DIVMTQSPSSLAMSVGQKVTMSCKSSQSLLNSKNQKNYLAWYQQKPGQSPKFLVYFAFFRESGVPDRFIGSGSGTDFTLTISSVQAEDLADYFCQQHYSTPFTFGSGTKLEIK

SEQ ID NO: 64 - protein, 112 aa, Artificial Sequence
  TM-82 ANTIBODY VARIABLE LIGHT CHAIN
DVVVTQTPLSLPVSFGDQVSISCRSSQSLANTYGNTYLSWYLHTPGQSPQLLIFGISNRFSGVPDRFSGSGSGTDFTLKISTIKPEDLGVYYCLQGTHPPRTFGGGTKLEMK

SEQ ID NO: 65 - protein, 112 aa, Artificial Sequence
  TM-83 ANTIBODY VARIABLE LIGHT CHAIN
DVVMTQTPLSLPVSLGDQASISCRSSQSLVHSNGITYLHWYLQKPGQSPKLLIYKVSNRFSGVPDRFSGSGSGTDFTLKISRVEAEDLGVYFCSQSTHVPYTFGGGTKLEIK

SEQ ID NO: 66 - protein, 107 aa, Artificial Sequence
  TM-84 ANTIBODY VARIABLE LIGHT CHAIN
DIQMTQSPASLSVSVGETVTITCRASENIYSSLGWYHQKQGKSPQLLVFAATNLADGVPSRFSGSGSGTQYSLKINSLQSEDFGTYYCQHLWSVPWTFGGGTKLEIK

SEQ ID NO: 67 - protein, 107 aa, Artificial Sequence
  TM-85 ANTIBODY VARIABLE LIGHT CHAIN
DIAMSQSQKFMSTSVGDRVSVTCKASQNVGTNVAWYQQKPGQSPKALIYSASYRYSGVPDRFTGSGSGTDFTLTISNVQSEDLAEYFCQQYNSYPLTFGAGTKLEMK

SEQ ID NO: 68 - protein, 113 aa, Artificial Sequence
  TM-86 ANTIBODY VARIABLE LIGHT CHAIN
DIVMTQSPSSLSVSAGEKVTMSCKSSQSLLNSGNQKNYLAWHQQRPGQPPKLLIYGASTRESGVPDRFTGSGSGTEFTLTISSVQAEDLAVYYCQNDHSYPLTFGAGTKLELK

SEQ ID NO: 69 - protein, 107 aa, Artificial Sequence
  TM-87 ANTIBODY VARIABLE LIGHT CHAIN
DVQITQSPSYLAASPGETITINCRASRSISKYLAWYQEKPGKTNKLLIYSGSTLQSGIPSRFSGSGSGTDFTLTISSLEPEDFAVYYCQQHNEYPLTFGAGTKLELK

SEQ ID NO: 70 - protein, 107 aa, Artificial Sequence
  TM-88 ANTIBODY VARIABLE LIGHT CHAIN
ETTVTQSPASLSMAIGEKVTIRCITSSDIDDHMNWYRQKPGEPPEFLISEGNALRPGVPSRFSSSGYGTDFIFTIENILSEDVADYYCLQSDNLPLTFGAGTKLELK

SEQ ID NO: 71 - protein, 107 aa, Artificial Sequence
  TM-89 ANTIBODY VARIABLE LIGHT CHAIN
DIQMTQSPSSLSASLGERVSLTCRASQDIGSSLNWLQQEPDGTIKRLIYATSSLDSGVPKRFSGSRSGSDYSLTISSLESEDFVDYYCLQYASSPRTFGGGTKLEIK

SEQ ID NO: 72 - protein, 112 aa, Artificial Sequence
  TM-90 ANTIBODY VARIABLE LIGHT CHAIN
DVLMTQTPLSLPVSLGDQASISCRSSQSFVHGNGNTYLEWYLQKPGQSPKLLIYKVSHRFSGVPDRFSGSGSGTDFTLKITRVEAEDLGVYYCFQNSHVPHTFGGGTKLEIK

SEQ ID NO: 73 - protein, 107 aa, Artificial Sequence
  TM-91 ANTIBODY VARIABLE LIGHT CHAIN
DIVMTQSQKFMSTSVGDRVSVTCKASQNVYTNVAWYQQKPGQSPKPLIYSASYRYSGVPDRFTGSGSGTDFTLTISNVQSEDLAEYFCQQYNSYPLTFGGGTKLEIK

SEQ ID NO: 74 - protein, 107 aa, Artificial Sequence
  TM-92 ANTIBODY VARIABLE LIGHT CHAIN
DVQITQSPSYLAASPGETITINCRASKSISKYLAWYQEKPGKTNKLLIYSGSTLQSGIPSRFSGSGSGTDFTLTISSLEPEDFAMYYCQQHNEYPWTFGGGTKLEIK

SEQ ID NO: 75 - protein, 107 aa, Artificial Sequence
  TM-93 ANTIBODY VARIABLE LIGHT CHAIN
DIVMTQSHKLMSTSVGDRVSITCKASQDVGSAVAWYQQKPGQSPQLLIYWSSTRLPGVPDRFTGSGSGTDFTLTISNVQSEVLADYFCQQYTSYPLTFGGGTKLEIK

SEQ ID NO: 76 - protein, 111 aa, Artificial Sequence
  TM-94 ANTIBODY VARIABLE LIGHT CHAIN
DVVMTQTPLSLPVSLGDQASISCRSSQSLVYSNGNTYLHWYLQRPGQSPKLLIYKVSNRFSGVPDRFSGSGSGTDFTLKISRVEAEDLGVYFCSQSTHVYTFGGGTKLEIK

SEQ ID NO: 77 - protein, 5 aa, Artificial Sequence
  ANTIBODY CDR-H1
TYDLN

SEQ ID NO: 78 - protein, 5 aa, Artificial Sequence
  ANTIBODY CDR-H1
SYWMH

SEQ ID NO: 79 - protein, 5 aa, Artificial Sequence
  ANTIBODY CDR-H1
SYDIN

SEQ ID NO: 80 - protein, 5 aa, Artificial Sequence
  ANTIBODY CDR-H1
NYWMN

SEQ ID NO: 81 - protein, 5 aa, Artificial Sequence
  ANTIBODY CDR-H1
RYWMH

SEQ ID NO: 82 - protein, 5 aa, Artificial Sequence
  ANTIBODY CDR-H1
SFWMH

SEQ ID NO: 83 - protein, 5 aa, Artificial Sequence
  ANTIBODY CDR-H1
SSWMH

SEQ ID NO: 84 - protein, 5 aa, Artificial Sequence
  ANTIBODY CDR-H1
NYAMS

SEQ ID NO: 85 - protein, 5 aa, Artificial Sequence
  ANTIBODY CDR-H1
DDYMH

SEQ ID NO: 86 - protein, 5 aa, Artificial Sequence
  ANTIBODY CDR-H1
SYGVH

SEQ ID NO: 87 - protein, 5 aa, Artificial Sequence
  ANTIBODY CDR-H1
NYWMH

SEQ ID NO: 88 - protein, 5 aa, Artificial Sequence
  ANTIBODY CDR-H1
SHWMH

SEQ ID NO: 89 - protein, 5 aa, Artificial Sequence
  ANTIBODY CDR-H1
SYVIH

SEQ ID NO: 90 - protein, 5 aa, Artificial Sequence
  ANTIBODY CDR-H1
SYVMH

SEQ ID NO: 91 - protein, 7 aa, Artificial Sequence
  ANTIBODY CDR-H1
TSGMGVS

SEQ ID NO: 92 - protein, 5 aa, Artificial Sequence
  ANTIBODY CDR-H1
DYEMH

SEQ ID NO: 93 - protein, 5 aa, Artificial Sequence
  ANTIBODY CDR-H1
NHWMH

SEQ ID NO: 94 - protein, 5 aa, Artificial Sequence
  ANTIBODY CDR-H1
NYVMH

SEQ ID NO: 95 - protein, 5 aa, Artificial Sequence
  ANTIBODY CDR-H1
TFWMH

SEQ ID NO: 96 - protein, 5 aa, Artificial Sequence
  ANTIBODY CDR-H1
NYLIE

SEQ ID NO: 97 - protein, 5 aa, Artificial Sequence
  ANTIBODY CDR-H1
TYWIH

SEQ ID NO: 98 - protein, 5 aa, Artificial Sequence
  ANTIBODY CDR-H1
TFPIE

SEQ ID NO: 99 - protein, 5 aa, Artificial Sequence
  ANTIBODY CDR-H1
GYWMH

SEQ ID NO: 100 - protein, 5 aa, Artificial Sequence
  ANTIBODY CDR-H1
DYYIH

SEQ ID NO: 101 - protein, 5 aa, Artificial Sequence
  ANTIBODY CDR-H1
DYYMN

SEQ ID NO: 102 - protein, 5 aa, Artificial Sequence
  ANTIBODY CDR-H1
DYYIN

SEQ ID NO: 103 - protein, 6 aa, Artificial Sequence
  ANTIBODY CDR-H1
SGYYWD

SEQ ID NO: 104 - protein, 5 aa, Artificial Sequence
  ANTIBODY CDR-H1
DYTMD

SEQ ID NO: 105 - protein, 5 aa, Artificial Sequence
  ANTIBODY CDR-H1
SYGIS

SEQ ID NO: 106 - protein, 5 aa, Artificial Sequence
  ANTIBODY CDR-H1
DYNMD

SEQ ID NO: 107 - protein, 17 aa, Artificial Sequence
  ANTIBODY CDR-H2
WIYPRGGSTEYNEKFKG

SEQ ID NO: 108 - protein, 17 aa, Artificial Sequence
  ANTIBODY CDR-H2
YINPSSGYSKYNQRFKD

SEQ ID NO: 109 - protein, 17 aa, Artificial Sequence
  ANTIBODY CDR-H2
YVNPSSGYTKNNQKFKD

SEQ ID NO: 110 - protein, 17 aa, Artificial Sequence
  ANTIBODY CDR-H2
WIYPRDGNTQYIEKLKG

SEQ ID NO: 111 - protein, 17 aa, Artificial Sequence
  ANTIBODY CDR-H2
YINPSSDYTKYNQNFKD

SEQ ID NO: 112 - protein, 17 aa, Artificial Sequence
  ANTIBODY CDR-H2
NIDPSDSETQYNPKFKD

SEQ ID NO: 113 - protein, 17 aa, Artificial Sequence
  ANTIBODY CDR-H2
NIDPSDSETHYSQKFKD

SEQ ID NO: 114 - protein, 17 aa, Artificial Sequence
  ANTIBODY CDR-H2
NIDPSDSDTHYNQNFRG

SEQ ID NO: 115 - protein, 17 aa, Artificial Sequence
  ANTIBODY CDR-H2
EINPKNGGTNYNEKFKT

SEQ ID NO: 116 - protein, 17 aa, Artificial Sequence
  ANTIBODY CDR-H2
TIGSVGSYTYYPDSVKG

SEQ ID NO: 117 - protein, 17 aa, Artificial Sequence
  ANTIBODY CDR-H2
WIDPENGDTEYASKFQG

SEQ ID NO: 118 - protein, 16 aa, Artificial Sequence
  ANTIBODY CDR-H2
IMGWDDKKYYNSALKS

SEQ ID NO: 119 - protein, 17 aa, Artificial Sequence
  ANTIBODY CDR-H2
TIGYGGSYSYYPDSVKG

SEQ ID NO: 120 - protein, 17 aa, Artificial Sequence
  ANTIBODY CDR-H2
NIDPSDSDTHYNQKFKD

SEQ ID NO: 121 - protein, 17 aa, Artificial Sequence
  ANTIBODY CDR-H2
EIDPSDSYTYYNQKFKG

SEQ ID NO: 122 - protein, 17 aa, Artificial Sequence
  ANTIBODY CDR-H2
FINPNNDGTKYNEKFRG

SEQ ID NO: 123 - protein, 17 aa, Artificial Sequence
  ANTIBODY CDR-H2
YINPYNDGTKYNEKFKG

SEQ ID NO: 124 - protein, 16 aa, Artificial Sequence
  ANTIBODY CDR-H2
HIFWDDDKRYNLFLKS

SEQ ID NO: 125 - protein, 17 aa, Artificial Sequence
  ANTIBODY CDR-H2
VIDPETGGTAYNQMFKG

SEQ ID NO: 126 - protein, 17 aa, Artificial Sequence
  ANTIBODY CDR-H2
NIDPSDSDTHYNHKFKD

SEQ ID NO: 127 - protein, 17 aa, Artificial Sequence
  ANTIBODY CDR-H2
HITPHNDGTKYNEKFKG

SEQ ID NO: 128 - protein, 17 aa, Artificial Sequence
  ANTIBODY CDR-H2
NIDPSDSETHYNQEFKD

SEQ ID NO: 129 - protein, 17 aa, Artificial Sequence
  ANTIBODY CDR-H2
NIDPSDSEIHYNQKFKD

SEQ ID NO: 130 - protein, 17 aa, Artificial Sequence
  ANTIBODY CDR-H2
VINPGSGITDYSEKFKG

SEQ ID NO: 131 - protein, 17 aa, Artificial Sequence
  ANTIBODY CDR-H2
NINPSNGNTKNNERFKT

SEQ ID NO: 132 - protein, 17 aa, Artificial Sequence
  ANTIBODY CDR-H2
NFHPYNDDTKYNEKFKG

SEQ ID NO: 133 - protein, 17 aa, Artificial Sequence
  ANTIBODY CDR-H2
RIDPYSGDTRYNEKFKN

SEQ ID NO: 134 - protein, 17 aa, Artificial Sequence
  ANTIBODY CDR-H2
VISPYNDNTNYNQKFKG

SEQ ID NO: 135 - protein, 17 aa, Artificial Sequence
  ANTIBODY CDR-H2
VINPYNGATSYNQKFQD

SEQ ID NO: 136 - protein, 17 aa, Artificial Sequence
  ANTIBODY CDR-H2
VINPYSGATYYTQKFKG

SEQ ID NO: 137 - protein, 17 aa, Artificial Sequence
  ANTIBODY CDR-H2
VINPGSGGTDYNEKFKG

SEQ ID NO: 138 - protein, 17 aa, Artificial Sequence
  ANTIBODY CDR-H2
WIYPRDGNAKYNEKFKG

SEQ ID NO: 139 - protein, 16 aa, Artificial Sequence
  ANTIBODY CDR-H2
YISYDGNNNYNPSLKN

SEQ ID NO: 140 - protein, 17 aa, Artificial Sequence
  ANTIBODY CDR-H2
HINPNDGGTFYNQKFKG

SEQ ID NO: 141 - protein, 16 aa, Artificial Sequence
  ANTIBODY CDR-H2
VIWTGGGTNYNSALES

SEQ ID NO: 142 - protein, 17 aa, Artificial Sequence
  ANTIBODY CDR-H2
DINPYNGGTLYNQKFKG

SEQ ID NO: 143 - protein, 8 aa, Artificial Sequence
  ANTIBODY CDR-H3
YSIYAMDY

SEQ ID NO: 144 - protein, 12 aa, Artificial Sequence
  ANTIBODY CDR-H3
EGGSISDWYFDV

SEQ ID NO: 145 - protein, 8 aa, Artificial Sequence
  ANTIBODY CDR-H3
WIFYAMDY

SEQ ID NO: 146 - protein, 12 aa, Artificial Sequence
  ANTIBODY CDR-H3
WGGSRSYWYFDV

SEQ ID NO: 147 - protein, 10 aa, Artificial Sequence
  ANTIBODY CDR-H3
LTTLDYAMDY

SEQ ID NO: 148 - protein, 9 aa, Artificial Sequence
  ANTIBODY CDR-H3
SSNWDNFDY

SEQ ID NO: 149 - protein, 14 aa, Artificial Sequence
  ANTIBODY CDR-H3
SRGGTYDYGEAMDY

SEQ ID NO: 150 - protein, 6 aa, Artificial Sequence
  ANTIBODY CDR-H3
PTWFTY

SEQ ID NO: 151 - protein, 9 aa, Artificial Sequence
  ANTIBODY CDR-H3
QEGSSHFDY

SEQ ID NO: 152 - protein, 7 aa, Artificial Sequence
  ANTIBODY CDR-H3
PQTATTC

SEQ ID NO: 153 - protein, 9 aa, Artificial Sequence
  ANTIBODY CDR-H3
EAGPYGMDY

SEQ ID NO: 154 - protein, 12 aa, Artificial Sequence
  ANTIBODY CDR-H3
QDSNYEGVWFAY

SEQ ID NO: 155 - protein, 14 aa, Artificial Sequence
  ANTIBODY CDR-H3
SRGGYYDYGEAMDY

SEQ ID NO: 156 - protein, 11 aa, Artificial Sequence
  ANTIBODY CDR-H3
NYHGSGPPFAH

SEQ ID NO: 157 - protein, 12 aa, Artificial Sequence
  ANTIBODY CDR-H3
PYYDYEGYTMDY

SEQ ID NO: 158 - protein, 10 aa, Artificial Sequence
  ANTIBODY CDR-H3
GGWFPYAMDY

SEQ ID NO: 159 - protein, 10 aa, Artificial Sequence
  ANTIBODY CDR-H3
MGRQRDYFDY

SEQ ID NO: 160 - protein, 6 aa, Artificial Sequence
  ANTIBODY CDR-H3
YGIEGY

SEQ ID NO: 161 - protein, 12 aa, Artificial Sequence
  ANTIBODY CDR-H3
SGQFGRGDYFHY

SEQ ID NO: 162 - protein, 9 aa, Artificial Sequence
  ANTIBODY CDR-H3
SPAGQILDY

SEQ ID NO: 163 - protein, 11 aa, Artificial Sequence
  ANTIBODY CDR-H3
SPFITTVVADY

SEQ ID NO: 164 - protein, 10 aa, Artificial Sequence
  ANTIBODY CDR-H3
GDYYGTSYPF

SEQ ID NO: 165 - protein, 8 aa, Artificial Sequence
  ANTIBODY CDR-H3
YFYGGMDY

SEQ ID NO: 166 - protein, 9 aa, Artificial Sequence
  ANTIBODY CDR-H3
YGLGHAMDY

SEQ ID NO: 167 - protein, 9 aa, Artificial Sequence
  ANTIBODY CDR-H3
TYYSNYFDY

SEQ ID NO: 168 - protein, 9 aa, Artificial Sequence
  ANTIBODY CDR-H3
GNYDWYFDV

SEQ ID NO: 169 - protein, 12 aa, Artificial Sequence
  ANTIBODY CDR-H3
GGYDYGHWYFDV

SEQ ID NO: 170 - protein, 13 aa, Artificial Sequence
  ANTIBODY CDR-H3
DPYSKYVHYPMDY

SEQ ID NO: 171 - protein, 12 aa, Artificial Sequence
  ANTIBODY CDR-H3
VSDYHGGYGMDY

SEQ ID NO: 172 - protein, 9 aa, Artificial Sequence
  ANTIBODY CDR-H3
AVYSVGFAY

SEQ ID NO: 173 - protein, 16 aa, Artificial Sequence
  ANTIBODY CDR-H3
WVYYYGISYEGGAMDY

SEQ ID NO: 174 - protein, 15 aa, Artificial Sequence
  ANTIBODY CDR-H3
NPPKIFYDYLMYFDY

SEQ ID NO: 175 - protein, 9 aa, Artificial Sequence
  ANTIBODY CDR-H3
VGYHGTPDY

SEQ ID NO: 176 - protein, 16 aa, Artificial Sequence
  ANTIBODY CDR-L1
RSSQSIVHGNGNTYLE

SEQ ID NO: 177 - protein, 16 aa, Artificial Sequence
  ANTIBODY CDR-L1
RSSKSLLQNNGNTYLY

SEQ ID NO: 178 - protein, 16 aa, Artificial Sequence
  ANTIBODY CDR-L1
RSSKSLLHSNGNTYSY

SEQ ID NO: 179 - protein, 17 aa, Artificial Sequence
  ANTIBODY CDR-L1
KSSQSLLNSGNQKNYLA

SEQ ID NO: 180 - protein, 10 aa, Artificial Sequence
  ANTIBODY CDR-L1
SASSSVSYMY

SEQ ID NO: 181 - protein, 16 aa, Artificial Sequence
  ANTIBODY CDR-L1
RSSQSLLHVNGNTYLY

SEQ ID NO: 182 - protein, 16 aa, Artificial Sequence
  ANTIBODY CDR-L1
RSSKSLLHTNGNTYLF

SEQ ID NO: 183 - protein, 11 aa, Artificial Sequence
  ANTIBODY CDR-L1
RASQNINIWLS

SEQ ID NO: 184 - protein, 16 aa, Artificial Sequence
  ANTIBODY CDR-L1
RSSQSLVNSYGKTFLS

SEQ ID NO: 185 - protein, 16 aa, Artificial Sequence
  ANTIBODY CDR-L1
SSSKSLLHSSGITYLY

SEQ ID NO: 186 - protein, 16 aa, Artificial Sequence
  ANTIBODY CDR-L1
RSSQSLVHSNGNTYLH

SEQ ID NO: 187 - protein, 15 aa, Artificial Sequence
  ANTIBODY CDR-L1
RASESVDNYGISFMN

SEQ ID NO: 188 - protein, 10 aa, Artificial Sequence
  ANTIBODY CDR-L1
SASSGISYIY

SEQ ID NO: 189 - protein, 11 aa, Artificial Sequence
  ANTIBODY CDR-L1
KASQNVRSAVA

SEQ ID NO: 190 - protein, 15 aa, Artificial Sequence
  ANTIBODY CDR-L1
RASQSVDYNGISYMH

SEQ ID NO: 191 - protein, 10 aa, Artificial Sequence
  ANTIBODY CDR-L1
SASLSISYVH

SEQ ID NO: 192 - protein, 16 aa, Artificial Sequence
  ANTIBODY CDR-L1
KSSQSLFHSNGKTYLN

SEQ ID NO: 193 - protein, 16 aa, Artificial Sequence
  ANTIBODY CDR-L1
RSSKSLLHVNGNTYLF

SEQ ID NO: 194 - protein, 15 aa, Artificial Sequence
  ANTIBODY CDR-L1
RASKSVSTSGSIYIH

SEQ ID NO: 195 - protein, 16 aa, Artificial Sequence
  ANTIBODY CDR-L1
RSSQSLLHVNGHTYLY

SEQ ID NO: 196 - protein, 17 aa, Artificial Sequence
  ANTIBODY CDR-L1
KSSQSLLNSKNQKNYLA

SEQ ID NO: 197 - protein, 16 aa, Artificial Sequence
  ANTIBODY CDR-L1
RSSQSLANTYGNTYLS

SEQ ID NO: 198 - protein, 16 aa, Artificial Sequence
  ANTIBODY CDR-L1
RSSQSLVHSNGITYLH

SEQ ID NO: 199 - protein, 11 aa, Artificial Sequence
  ANTIBODY CDR-L1
RASENIYSSLG

SEQ ID NO: 200 - protein, 11 aa, Artificial Sequence
  ANTIBODY CDR-L1
KASQNVGTNVA

SEQ ID NO: 201 - protein, 11 aa, Artificial Sequence
  ANTIBODY CDR-L1
RASRSISKYLA

SEQ ID NO: 202 - protein, 11 aa, Artificial Sequence
  ANTIBODY CDR-L1
ITSSDIDDHMN

SEQ ID NO: 203 - protein, 11 aa, Artificial Sequence
  ANTIBODY CDR-L1
RASQDIGSSLN

SEQ ID NO: 204 - protein, 16 aa, Artificial Sequence
  ANTIBODY CDR-L1
RSSQSFVHGNGNTYLE

SEQ ID NO: 205 - protein, 11 aa, Artificial Sequence
  ANTIBODY CDR-L1
KASQNVYTNVA

SEQ ID NO: 206 - protein, 11 aa, Artificial Sequence
  ANTIBODY CDR-L1
RASKSISKYLA

SEQ ID NO: 207 - protein, 11 aa, Artificial Sequence
  ANTIBODY CDR-L1
KASQDVGSAVA

SEQ ID NO: 208 - protein, 16 aa, Artificial Sequence
  ANTIBODY CDR-L1
RSSQSLVYSNGNTYLH

SEQ ID NO: 209 - protein, 7 aa, Artificial Sequence
  ANTIBODY CDR-L2
KVSNRFS

SEQ ID NO: 210 - protein, 7 aa, Artificial Sequence
  ANTIBODY CDR-L2
RMSNLAS

SEQ ID NO: 211 - protein, 7 aa, Artificial Sequence
  ANTIBODY CDR-L2
GASTRES

SEQ ID NO: 212 - protein, 7 aa, Artificial Sequence
  ANTIBODY CDR-L2
RTSNLAS

SEQ ID NO: 213 - protein, 7 aa, Artificial Sequence
  ANTIBODY CDR-L2
KASNLHT

SEQ ID NO: 214 - protein, 7 aa, Artificial Sequence
  ANTIBODY CDR-L2
GISNRFS

SEQ ID NO: 215 - protein, 7 aa, Artificial Sequence
  ANTIBODY CDR-L2
AASNQGS

SEQ ID NO: 216 - protein, 7 aa, Artificial Sequence
  ANTIBODY CDR-L2
RKSNLAS

SEQ ID NO: 217 - protein, 7 aa, Artificial Sequence
  ANTIBODY CDR-L2
DTSNLAS

SEQ ID NO: 218 - protein, 7 aa, Artificial Sequence
  ANTIBODY CDR-L2
LASNRHT

SEQ ID NO: 219 - protein, 7 aa, Artificial Sequence
  ANTIBODY CDR-L2
AASNLES

SEQ ID NO: 220 - protein, 7 aa, Artificial Sequence
  ANTIBODY CDR-L2
GTSNLAS

SEQ ID NO: 221 - protein, 7 aa, Artificial Sequence
  ANTIBODY CDR-L2
MVSKLES

SEQ ID NO: 222 - protein, 7 aa, Artificial Sequence
  ANTIBODY CDR-L2
LTSNLES

SEQ ID NO: 223 - protein, 7 aa, Artificial Sequence
  ANTIBODY CDR-L2
FAFFRES

SEQ ID NO: 224 - protein, 7 aa, Artificial Sequence
  ANTIBODY CDR-L2
GISNRFS

SEQ ID NO: 225 - protein, 7 aa, Artificial Sequence
  ANTIBODY CDR-L2
AATNLAD

SEQ ID NO: 226 - protein, 7 aa, Artificial Sequence
  ANTIBODY CDR-L2
SASYRYS

SEQ ID NO: 227 - protein, 7 aa, Artificial Sequence
  ANTIBODY CDR-L2
SGSTLQS

SEQ ID NO: 228 - protein, 7 aa, Artificial Sequence
  ANTIBODY CDR-L2
EGNALRP

SEQ ID NO: 229 - protein, 7 aa, Artificial Sequence
  ANTIBODY CDR-L2
ATSSLDS

SEQ ID NO: 230 - protein, 7 aa, Artificial Sequence
  ANTIBODY CDR-L2
KVSHRFS

SEQ ID NO: 231 - protein, 7 aa, Artificial Sequence
  ANTIBODY CDR-L2
WSSTRLP

SEQ ID NO: 232 - protein, 9 aa, Artificial Sequence
  ANTIBODY CDR-L3
FQGSHVPWT

SEQ ID NO: 233 - protein, 9 aa, Artificial Sequence
  ANTIBODY CDR-L3
MQHLEYPYT

SEQ ID NO: 234 - protein, 9 aa, Artificial Sequence
  ANTIBODY CDR-L3
FQGSHLPYT

SEQ ID NO: 235 - protein, 9 aa, Artificial Sequence
  ANTIBODY CDR-L3
QNDSNYPFT

SEQ ID NO: 236 - protein, 9 aa, Artificial Sequence
  ANTIBODY CDR-L3
QQYHSYPPT

SEQ ID NO: 237 - protein, 9 aa, Artificial Sequence
  ANTIBODY CDR-L3
MQHLQYPFS

SEQ ID NO: 238 - protein, 9 aa, Artificial Sequence
  ANTIBODY CDR-L3
MQHLEYPFT

SEQ ID NO: 239 - protein, 9 aa, Artificial Sequence
  ANTIBODY CDR-L3
LQGQSYPYT

SEQ ID NO: 240 - protein, 9 aa, Artificial Sequence
  ANTIBODY CDR-L3
LQGTHPPLT

SEQ ID NO: 241 - protein, 9 aa, Artificial Sequence
  ANTIBODY CDR-L3
AQMLERPWT

SEQ ID NO: 242 - protein, 9 aa, Artificial Sequence
  ANTIBODY CDR-L3
SQNTHVPLT

SEQ ID NO: 243 - protein, 9 aa, Artificial Sequence
  ANTIBODY CDR-L3
QQSKDVPWT

SEQ ID NO: 244 - protein, 9 aa, Artificial Sequence
  ANTIBODY CDR-L3
HQWRSYPPT

SEQ ID NO: 245 - protein, 9 aa, Artificial Sequence
  ANTIBODY CDR-L3
LQHWKYPFT

SEQ ID NO: 246 - protein, 9 aa, Artificial Sequence
  ANTIBODY CDR-L3
QQSIEDPWT

SEQ ID NO: 247 - protein, 8 aa, Artificial Sequence
  ANTIBODY CDR-L3
HQWSIYRT

SEQ ID NO: 248 - protein, 9 aa, Artificial Sequence
  ANTIBODY CDR-L3
LQVTHFPLT

SEQ ID NO: 249 - protein, 9 aa, Artificial Sequence
  ANTIBODY CDR-L3
QHSGELPWT

SEQ ID NO: 250 - protein, 9 aa, Artificial Sequence
  ANTIBODY CDR-L3
MQHLQYPFT

SEQ ID NO: 251 - protein, 9 aa, Artificial Sequence
  ANTIBODY CDR-L3
QQHYSTPFT

SEQ ID NO: 252 - protein, 9 aa, Artificial Sequence
  ANTIBODY CDR-L3
LQGTHPPRT

SEQ ID NO: 253 - protein, 9 aa, Artificial Sequence
  ANTIBODY CDR-L3
SQSTHVPYT

SEQ ID NO: 254 - protein, 9 aa, Artificial Sequence
  ANTIBODY CDR-L3
QHLWSVPWT

SEQ ID NO: 255 - protein, 9 aa, Artificial Sequence
  ANTIBODY CDR-L3
QQYNSYPLT

SEQ ID NO: 256 - protein, 9 aa, Artificial Sequence
  ANTIBODY CDR-L3
QNDHSYPLT

SEQ ID NO: 257 - protein, 9 aa, Artificial Sequence
  ANTIBODY CDR-L3
QQHNEYPLT

SEQ ID NO: 258 - protein, 9 aa, Artificial Sequence
  ANTIBODY CDR-L3
LQSDNLPLT

SEQ ID NO: 259 - protein, 9 aa, Artificial Sequence
  ANTIBODY CDR-L3
LQYASSPRT

SEQ ID NO: 260 - protein, 9 aa, Artificial Sequence
  ANTIBODY CDR-L3
FQNSHVPHT

SEQ ID NO: 261 - protein, 9 aa, Artificial Sequence
  ANTIBODY CDR-L3
QQHNEYPWT

SEQ ID NO: 262 - protein, 9 aa, Artificial Sequence
  ANTIBODY CDR-L3
QQYTSYPLT

SEQ ID NO: 263 - protein, 8 aa, Artificial Sequence
  ANTIBODY CDR-L3
SQSTHVYT

SEQ ID NO: 264 - DNA, 19 bp, Artificial Sequence
  Heavy chain constant region primer
agctgggaaggtgtgcaca

SEQ ID NO: 265 - DNA, 21 bp, Artificial Sequence
  Light constant region primer
ccattttgtcgttcactgcca

SEQ ID NO: 266 - protein, 347 aa, Artificial Sequence
  GST FUSION HUMAN TMEM106B ECD (truncated)
MDMRVPAQLLGLLLLWLRGARCMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDPREFIVTDDIEGRMDPDVKYIGVKSAYVSYDVQKRTIYLNITNTLNITNNNYYSVEVENITAQVQFSKTVIGKARLNNITIIGPLDMKQIDYTVPTVIAEEMSYM

SEQ ID NO: 267 - protein, 415 aa, Artificial Sequence
  GST FUSION HUMAN TMEM106B ECD
MDMRVPAQLLGLLLLWLRGARCMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDPREFIVTDDIEGRMDPPRSIDVKYIGVKSAYVSYDVQKRTIYLNITNTLNITNNNYYSVEVENITAQVQFSKTVIGKARLNNITIIGPLDMKQIDYTVPTVIAEEMSYMYDFCTLISIKVHNIVLMMQVTVTTTYFGHSEQISQERYQYVDCGRNTTYQLGQSEYLNVLQPQQ